SEQUENCE LISTING (40 sequences)

SEQ ID NO: 1 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 14 aa, Homo sapiens
SKRNIQFSCKNIYR

SEQ ID NO: 3 - protein, 14 aa, Homo sapiens
EKVQTLEAWVIHGG

SEQ ID NO: 4 - protein, 122 aa, Homo sapiens [sequence omitted]

SEQ ID NO: 5 - protein, 107 aa, Homo sapiens [sequence omitted]

SEQ ID NO: 6 - protein, 5 aa, Homo sapiens
SFAMS

SEQ ID NO: 7 - protein, 17 aa, Homo sapiens
AISGSGGGTYYADSVKG

SEQ ID NO: 8 - protein, 13 aa, Homo sapiens
DKILWFGEPVFDY

SEQ ID NO: 9 - protein, 11 aa, Homo sapiens
RASQSVSSYLA

SEQ ID NO: 10 - protein, 7 aa, Homo sapiens
DASNRAT

SEQ ID NO: 11 - protein, 10 aa, Homo sapiens
QQRSNWPPTF

SEQ ID NO: 12 - protein, 452 aa, Homo sapiens
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 122 aa, Homo sapiens
QVQLVQSGAEVKKPGSSVKVSCKASGGTFSSYAFSWVRQAPGQGLEWMGRVIPFLGIANSAQKFQGRVTITADKSTSTAYMDLSSLRSEDTAVYYCARDDIAALGPFDYWGQGTLVTVSSAS

SEQ ID NO: 15 - protein, 107 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYPRTFGQGTKVEIK

SEQ ID NO: 16 - protein, 122 aa, Homo sapiens
EVQLVQSGAEVKKPGESLKISCKGSGYSFSNYWIGWVRQMPGKGLEWMGIIYPHDSDARYSPSFQGQVTFSADKSISTAYLQWSSLKASDTAMYYCARHVGWGSRYWYFDLWGRGTLVTVSS

SEQ ID NO: 17 - protein, 107 aa, Homo sapiens
EIVLTQSPATLSLSPGERATLSCRASQSVSSYLAWYQQKPGQAPGLLIYDASNRASGIPARFSGSGSGTDFTLTISSLEPEDFAVYYCQQRSNWPLTFGGGTKVEIK

SEQ ID NO: 18 - protein, 120 aa, Homo sapiens
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSS

SEQ ID NO: 19 - protein, 109 aa, Homo sapiens
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVLGQ

SEQ ID NO: 20 - protein, 120 aa, Homo sapiens [sequence omitted]

SEQ ID NO: 21 - protein, 107 aa, Homo sapiens [sequence omitted]

SEQ ID NO: 22 - protein, 447 aa, Homo sapiens [sequence omitted]

SEQ ID NO: 23 - protein, 120 aa, Homo sapiens [sequence omitted]

SEQ ID NO: 24 - protein, 111 aa, Homo sapiens [sequence omitted]

SEQ ID NO: 25 - protein, 113 aa, Homo sapiens [sequence omitted]

SEQ ID NO: 26 - protein, 107 aa, Homo sapiens [sequence omitted]

SEQ ID NO: 27 - protein, 121 aa, Homo sapiens
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 28 - protein, 108 aa, Homo sapiens
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 29 - protein, 118 aa, Homo sapiens [sequence omitted]

SEQ ID NO: 30 - protein, 107 aa, Homo sapiens [sequence omitted]

SEQ ID NO: 31 - protein, 120 aa, Homo sapiens
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 32 - protein, 110 aa, Homo sapiens
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 33 - protein, 123 aa, Homo sapiens
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSS

SEQ ID NO: 34 - protein, 107 aa, Homo sapiens
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIK

SEQ ID NO: 35 - protein, 117 aa, Homo sapiens
EVQLVESGGGLVQPGGSLRLSCAASGFAFSRYDMSWVRQAPGKGLESVAYISGGGANTYYLDNVKGRFTISRDNAKNSLYLQMNSLRAEDTAVYYCASPYLSYFDVWGQGTLVTVSS

SEQ ID NO: 36 - protein, 107 aa, Homo sapiens
EIVMTQSPATLSVSPGERATLSCRASQSLSDYLHWYQQKPGQAPRLLIKSASQSISGIPARFSGSGSGTEFTLTISSLQSEDFAVYYCQNGHSFPYTFGQGTKLEIK

SEQ ID NO: 37 - protein, 117 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPYAPLDYWGQGTLVTVSS

SEQ ID NO: 38 - protein, 107 aa, Homo sapiens
EIVLTQSPATLSLSPGERATLSCRASQSVNDYLAWYQQKPGQAPRLLIYDASNRATGIPARFSGSGSGTDFTLTISSLEPEDFAVYYCQQGGHAPITFGQGTKVEIK

SEQ ID NO: 39 - protein, 124 aa, Homo sapiens
EVQLVQSGAEVKKPGESLKISCKGSGYSFTSYWMQWVRQMPGKGLEWMGAIYPGDGDIRYTQNFKGQVTISADKSISTAYLQWSSLKASDTAMYYCARWEKSTTVVQRNYFDYWGQGTTVTVSS

SEQ ID NO: 40 - protein, 106 aa, Homo sapiens
DIQMTQSPSSLSASVGDRVTITCKASENVGTFVSWYQQKPGKAPKLLIYGASNRYTGVPSRFSGSGSGTDFTLTISSLQPEDFATYYCGQSYSYPTFGQGTKLEIK

(19) United States
(12) Patent Application Publication
(10) Pub. No.: US 2022/0275090 A1
(43) Pub. Date: Sep. 1, 2022

(54) Combination Therapies with Anti-CD38 Antibodies and PARP or Adenosine Receptor Inhibitors

(71) Applicant: Janssen Biotech, Inc., Horsham, PA (US)

(72) Inventor: Diana Alvarez Arias, North Wales, PA (US)

(21) Appl. No.: 17/674,397

(22) Filed: Feb. 17, 2022

Related U.S. Application Data
(60) Provisional application No. 63/151,924, filed on Feb. 22, 2021.

(51) Int. Cl. C07K 16/28; A61K 31/4439; A61K 31/502; A61P 35/00; A61K 31/55; A61K 31/5025

(52) U.S. Cl. CPC C07K 16/2818; A61K 31/4439; A61K 31/502; A61P 35/00; A61K 31/55; A61K 31/5025

(57) ABSTRACT

This invention relates to a methods and compositions for treating a disease by administering a combination therapy comprising an anti-CD38 antibody and a poly ADP ribose polymerase inhibitor (PARPi); an anti-CD38 antibody and an adenosine receptor antagonist; or an anti-CD38 antibody, a PARPi and an adenosine receptor antagonist, to a subject (e.g., a human patient) in need thereof.

### CROSS-REFERENCE TO RELATED APPLICATIONS

This application claims priority to U.S. Provisional Application Ser. No. 63/151,924, filed 22 Feb. 2021, the entire contents of which is incorporated herein by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file name “JBI6472USNP1SEQLIST.txt” creation date of Feb. 2, 2022 and having a size of 43 KB. The sequence listing submitted via EFS-Web is part of the specification and is herein incorporated by reference in its entirety.

### BACKGROUND

CD38 is a multifunctional protein having function in receptor-mediated adhesion and signaling as well as mediating calcium mobilization via its ecto-enzymatic activity, catalyzing formation of cyclic ADP-ribose (cADPR) and ADPR. CD38 mediates cytokine secretion and activation and proliferation of lymphocytes (Funaro et al., J Immunol. 145(8): 2390-96 (1990); Terhorst et al., Cell 23(3): 771-80 (1981); Guse et al., Nature 398: 70-73 (1999)). Because CD38 is expressed on various malignant cells, anti-CD38 antibodies are being developed for the treatment of malignancies such as multiple myeloma (MM) and light chain amyloidosis (AL). CD38 is the main mammalian enzyme that hydrolyzes nicotinamide adenine dinucleotide (NAD⁺), and regulates its extracellular levels. Accordingly, a patient treated with an anti-CD38 antibody may experience accumulation of NAD⁺ and decrease of adenosine.

NAD⁺ is an essential co-enzyme and a central signaling molecule involved in maintaining redox homeostasis, efficient signal transduction, and mitochondrial metabolism. The extracellular conversion of NAD⁺ can vary significantly according to the tissue environment or pathological conditions (Horenstein et al., Cells. 4(3): 520-37 (2015)).

As a substrate, NAD⁺ is converted to adenosine (ADO), which is taken up by the cells and transformed and reincorporated into the intracellular nucleotide pool (Id.). Adenosine is an important intermediary metabolite, acting as a building block for nucleic acids and a component of the biological energy currency ATP (Chen et al., Nat Rev Drug Discov. 12(4): 265-86 (2013)). Adenosine also functions as a signaling molecule through the activation of four distinct adenosine receptors, A₁, A_{2A}, A_{2B} and A₃. These receptors are widely expressed and have been implicated in cardiac rhythm, circulation, lipolysis, renal blood flow, immune function, sleep regulation and angiogenesis, as well as inflammatory diseases, ischemia-perfusion and neurodegenerative disorders (Id.).

### SUMMARY

There is a critical need to determine tissue- and age-specific effects of CD38 reduction in the levels of NAD⁺ and adenosine and to identify therapeutic agents that benefit patients treated with anti-CD38 antibodies (e.g., patients who have multiple myeloma (MM) or light chain amyloidosis (AL)).

The invention disclosed herein is based, at least in part, on the ability to determine tissue- and age-specific effects of CD38 on reduction of NAD⁺, cADPR and adenosine levels in a mammalian model. In some embodiments, the invention generally relates to methods of treating a disease or condition in a subject (e.g., a human patient) in need thereof.

In one aspect, the invention provides methods of treating a disease in a subject in need thereof, comprising administering to the subject an anti-CD38 antibody and a poly ADP ribose polymerase inhibitor (PARPi) for a time sufficient to treat the disease.

In some embodiments, the PARPi is a PARP1 inhibitor, a PARP2 inhibitor, a PARP3 inhibitor, a PARP4 inhibitor, a PARP5 inhibitor, a PARP6 inhibitor, a PARP7 inhibitor, a PARP8 inhibitor, a PARP9 inhibitor, a PARP10 inhibitor, a PARP11 inhibitor, a PARP12 inhibitor, a PARP13 inhibitor, a PARP14 inhibitor, a PARP15 inhibitor, a PARP16 inhibitor or a PARP17 inhibitor, or a combination thereof. In some embodiments, the PARPi is a PARP1 inhibitor, a PARP2 inhibitor or a PARP3 inhibitor, or a combination thereof. In some embodiments, the PARPi is AG-14361, AZD2461, CEP-8983, CEP-9722, E7016 (GPI21016), iniparib (BSI 201), INO-1001, niraparib (MK-4827), NU1025, olaparib (AZD-2281), pamiparib (BGB-290), PJ34, PJ34HC1, RBN-2397, rucaparib (AG-014699, PF-01367338), talazoparib (BMN-673) or veliparib (ABT-888), or a pharmaceutically acceptable salt thereof. In some embodiments, the PARPi is Niraparib (MK-4827), Olaparib (AZD-2281), Rucaparib (AG-014699, PF-01367338), or Talazoparib (BMN-673), or a pharmaceutically acceptable salt thereof.

In another aspect, the invention provides methods of treating a disease in a subject in need thereof, comprising administering to the subject an anti-CD38 antibody and an adenosine receptor antagonist for a time sufficient to treat the disease.

In some embodiments, the adenosine receptor antagonist is an A₁ receptor (A₁AR) antagonist, an A_{2A} receptor (A_{2A}AR) antagonist, an A_{2B} receptor (A_{2B}AR) antagonist or an A₃ receptor (A₃AR) antagonist, or a combination thereof.

In some embodiments, the adenosine receptor antagonist is an A₁AR antagonist. In some embodiments, the A₁AR antagonist is BG 9719, DPCPX, FK453, FR194921, N-0861, rolofylline (KW 3902), tonapofylline (BG 9928) or WRC-0571.

In some embodiments, the adenosine receptor antagonist is an A_{2A}AR antagonist. In some embodiments, the A_{2A}AR antagonist is caffeine, 8-(-3-chlorostyryl)-caffeine (CSC), istradefylline (KW-6002), Preladenant (SCH 420814), “Schering compound” (see, e.g., Jacobson & Gao, Nat Rev Drug Discov., 5(3):247-64 (2006)), SCH 58261, SCH 442416, SYN115, VER 6947, VER 7835 or ZM241, 385.

In some embodiments, the adenosine receptor antagonist is an A_{2B}AR antagonist. In some embodiments, the A_{2B}AR antagonist is “Eisai compound” (see, e.g., Jacobson & Gao, Nat Rev Drug Discov., 5(3):247-64 (2006)), MRE 2029-F20, MRS1754 or OSIP-339391.

In some embodiments, the adenosine receptor antagonist is an A₃AR antagonist. In some embodiments, the A₃AR antagonist is FA385, MRE 3008-F20, MRS1292, MRS1334, MRS1523, MRS3777, “Novartis compound” (see, e.g., Jacobson & Gao, Nat Rev Drug Discov., 5(3):247-64 (2006)), OT-7999, PSB-11 or VUF5574.

In some embodiments, the anti-CD38 antibody comprises:
a) a heavy chain complementarity determining region 1 (HCDR1), HCDR2 and HCDR3 amino acid sequences of SEQ ID NOs: 6, 7 and 8, respectively; and
b) a light chain complementarity determining region 1 (LCDR1), LCDR2 and LCDR3 amino acid sequences of SEQ ID NOs: 9, 10 and 11, respectively.

In some embodiments, the anti-CD38 antibody comprises:
a) a heavy chain variable region (VH) amino acid sequence of SEQ ID NO: 4; and
b) a light chain variable region (VL) amino acid sequence of SEQ ID NO: 5.

In some embodiments, the anti-CD38 antibody comprises a heavy chain amino acid sequence of SEQ ID NO: 12 and a light chain amino acid sequence of SEQ ID NO: 13.

In some embodiments, the anti-CD38 antibody is of the IgG1, IgG2, IgG3 or IgG4 subtype. In some embodiments, the anti-CD38 antibody is of the IgG1 subtype.

In some embodiments, the anti-CD38 antibody is daratumumab.

In some embodiments, the anti-CD38 antibody is HexaBody-CD38 (GEN3014).

In some embodiments, the disease is cancer. In some embodiments, the cancer is a CD38-positive cancer. In some embodiments, the cancer is a CD38-negative cancer. In some embodiments, the cancer is a hematologic cancer. In some embodiments, the hematologic cancer is a CD38-positive hematological malignancy. In some embodiments, the hematologic cancer is multiple myeloma (MM). In some embodiments, the cancer is light chain amyloidosis (AL). In some embodiments, the cancer is a solid tumor. In some embodiments, the solid tumor is a CD38-positive solid tumor. In some embodiments, the solid tumor is a CD38-negative solid tumor. In some embodiments, the solid tumor is a metastatic lesion of the cancer.

In some embodiments, the disease is a neurological disorder. In some embodiments, the neurological disorder is Alzheimer's Disease (AD) or multiple sclerosis (MS).

In some embodiments, the disease is a liver disease. In some embodiments, the liver disease is non-alcoholic steatohepatitis (NASH).

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-1C show generation and validation of the CD38-KO mouse line on C57BL/6N background. FIG. 1A depicts the generation of the CD38-KO mouse line. Mouse CD38 expression was disrupted by inserting humanized CD38 flanked by loxP sites and subsequent Cre-mediated excision of the foxed region in vivo. FIG. 1B shows that mouse CD38 was not detected on immune subsets of CD38-KO mice. FIG. 1C shows that human CD38 was absent from B and NK cells of CD38-KO mice. PB: peripheral blood; SP: spleen; BM: bone marrow; FoB: follicular B cells; MZB: marginal zone B cells; iB: immature B cells.

FIGS. 2A-2E shows characteristics of the CD38-KO line. FIG. 2A shows that mature NKs and Tregs were modulated in CD38-KO mice. FIG. 2B shows T cell proportions in CD38-KO mice. FIG. 2C shows that B cell proportions were normal in CD38-KO mice. B220: total B220⁺ B cells; FoB: follicular B cells; MZB: marginal zone B cells; iB: immature B cells; T1B: transitional (from bone marrow) B cells; mB: mature B cells. FIG. 2D shows that the myeloid compartment was not affected in heterozygous (HT) and homozygous (HO) CD38-KO mice. CD38-KO mice. FIG. 2E shows that macrophage populations in CD38-KO mice varied in different organs. NS: non-significant >0.05; *: P≤0.05; **: P≤0.01; ****: P≤0.0001 (unpaired two-tailed t test).

FIGS. 3A-3B show that genetic disruption of CD38 increased NAD⁺ levels in various tissues of naïve non-tumor bearing mice. FIG. 3A compares young CD38-KO mice to young CD38-WT mice. FIG. 3B compares old CD38-KO mice to old CD38-WT mice. NS: non-significant >0.05; *: P≤0.05; **: P≤0.01; ****: P≤0.0001 (unpaired two-tailed t test).

FIGS. 4A-4D show that genetic disruption of CD38-mediated increase of NAD⁺ levels was age-dependent. FIGS. 4A and 4B compare tissue-specific changes in NAD⁺ levels between young and old mice. FIG. 4C compares NAD⁺ levels in old versus young CD38-WT mice. FIG. 4D compares NAD⁺ levels in old versus young CD38-KO mice. NS: non-significant >0.05; *: P≤0.05; **: P≤0.01; ****: P≤0.0001 (unpaired two-tailed t test).

FIGS. 5A-5D show that genetic disruption of CD38 altered adenosine levels in various tissues of naïve non-tumor bearing mice. FIGS. 5A and 5B compare young CD38-KO mice to young CD38-WT mice. FIGS. 5C and 5D compare old CD38-KO mice to old CD38-WT mice. NS: non-significant >0.05; *: P≤0.05; **: P≤0.01; ***: P≤0.001 (unpaired two-tailed t test).

FIGS. 6A-6C show that genetic disruption of CD38-mediated change of adenosine levels was age-dependent. FIG. 6A compares tissue-specific changes in adenosine levels between young and old mice. FIG. 6B compares adenosine levels in old versus young CD38-WT mice. FIG. 6C compares adenosine levels in old versus young CD38-KO mice. NS: non-significant >0.05; *: P≤0.05; **: P≤0.01; ***: P≤0.001; ****: P≤0.0001 (unpaired two-tailed t test).

FIG. 7 shows that genetic disruption of CD38 altered cADPR levels in various tissues of naïve non-tumor bearing young mice. ND: undetectable; *: P≤0.05; **: P≤0.01; ****: P≤0.0001 (unpaired two-tailed t test).

FIGS. 8A-8D show that CD38 is efficiently removed from splenic CD8 T cells (FIG. 8A), splenic CD4 T cells (FIG. 8B), tumor infiltrating T cells (TILs, FIG. 8C) and tumor cells (FIG. 8D) with the anti-CD38 NIMR5 mouse IgG2a antibody.

FIGS. 9A-9D show that treatment with the anti-CD38 NIMR5 mouse IgG2a antibody significantly increased NAD⁺ levels in the tissues and tumor. FIG. 9A shows anti-CD38 mediated increase in NAD⁺ levels in bone marrow, femur, lymph nodes, spleen and tumor. FIG. 9B shows an approximate 4-fold increase in the NAD⁺ level in the bone marrow isolated from right (R) femur in response to isotype treatment (Iso), anti-CD38 NIMR5 mouse IgG2a antibody (aCD38) treatment or genetic disruption of CD38 (KO). FIG. 9C shows that the increase in NAD⁺ levels in the tumors was smaller in the CD38-KO mice compared to mice treated with the anti-CD38 antibody, and that an active Fc (mouse IgG2a) was required for the anti-CD38 antibody to increase NAD⁺ levels. FIG. 9D shows that the increase in NAD⁺ levels in the femur and lymph nodes of mice treated with the anti-CD38 NIMR5 mouse IgG2a antibody and CD38-KO mice were of similar magnitude. NS: non-significant >0.05; *: P≤0.05; **: P≤0.01; ***: P≤0.001; ****: P≤0.0001 (unpaired two-tailed t test).

FIG. 10 compares NAD⁺ levels of the intact femurs (L, left) and bones without BMA (R, right). Lower NAD⁺ levels were detected in empty bone tissue, indicating that the main differences came from BMA.

FIGS. 11A-11C show that treatment with the anti-CD38 NIMR5 mouse IgG2a antibody did not significantly change the adenosine level in bone without BMA, femur, lymph nodes, spleen and tumors in young mice consistent with results in young naïve CD38 KO mice.

FIG. 12 shows that treatment with the anti-CD38 NIMR5 mouse IgG2a antibody decreased cADPR in all tissues tested, but did not reach statistical significance except in tumors. **: P≤0.01 (unpaired two-tailed t test).

### DETAILED DESCRIPTION

A description of example embodiments follows.

In one aspect, provided herein are methods of treating a disease in a subject in need thereof, comprising administering to the subject an anti-CD38 antibody and a poly ADP ribose polymerase inhibitor (PARPi) for a time sufficient to treat the disease.

In another aspect, provided herein are methods of treating a disease in a subject in need thereof, comprising administering to the subject an anti-CD38 antibody and an adenosine receptor antagonist for a time sufficient to treat the disease.

In another aspect, provided herein are methods of treating a disease in a subject in need thereof, comprising administering to the subject an anti-CD38 antibody, a PARPi and an adenosine receptor antagonist for a time sufficient to treat the disease.

### Anti-CD38 Antibodies

In some embodiments, the anti-CD38 antibody of the present invention binds human CD38 (SEQ ID NO: 1). In some embodiments, the anti-CD38 antibody binds at least to the region SKRNIQFSCKNIYR (SEQ ID NO: 2) and the region EKVQTLEAWVIHGG (SEQ ID NO: 3) of human CD38 (SEQ ID NO: 1). The amino acid sequences of SEQ ID NOs: 1-40 are provided in Table 1.

“CD38” refers to the human CD38 protein (synonyms include: ADP-ribosyl cyclase 1, cADPr hydrolase 1, cyclic ADP-ribose hydrolase 1). Human CD38 has an amino acid sequence shown in GenBank accession number NP_001766 and in SEQ ID NO: 1. Human CD38 is a single pass type II membrane protein with amino acid residues 1-21 representing the cytosolic domain, amino acid residues 22-42 representing the transmembrane domain, and amino acid residues 43-300 representing the extracellular domain.

In some embodiments, the anti-CD38 antibody comprises a heavy chain variable region (VH) amino acid sequence of SEQ ID NO: 4. In some embodiments, the anti-CD38 antibody comprises a VH amino acid sequence that is at least 95% identical, e.g., about: 95%, 96%, 97%, 98% or 99% identical to SEQ ID NO: 4.

In some embodiments, the anti-CD38 antibody comprises a light chain variable region (VL) amino acid sequence of SEQ ID NO: 5. In some embodiments, the anti-CD38 antibody comprises a VL amino acid sequence that is at least 95% identical, e.g., about: 95%, 96%, 97%, 98% or 99% identical to SEQ ID NO: 5.′

TABLE 1
Amino Acid Sequences

| SEQ | |
|---|---|
| ID | |
| NO: | Amino Acid Sequences |
| 1 | MANCEFSPVSGDKPCCRLSRRAQLCLGVSILVLILVVVLAVVVPRWRQQWSGPGTTKRFPET |
| | VLARCVKYTEIHPEMRHVDCQSVWDAFKGAFISKHPCNITEEDYQPLMKLGTQTVPCNKILL |
| | WSRIKDLAHQFTQVQRDMFTLEDTLLGYLADDLTWCGEFNTSKINYQSCPDWRKDCSNNPV |
| | SVFWKTVSRRFAEAACDVVHVMLNGSRSKIFDKNSTFGSVEVHNLQPEKVQTLEAWVIHGG |
| | REDSRDLCQDPTIKELESIISKRNIQFSCKNIYRPDKFLQCVKNPEDSSCTSEI |
| 2 | SKRNIQFSCKNIYR |
| 3 | EKVQTLEAWVIHGG |
| 4 | EVQLLESGGGLVQPGGSLRLSCAVSGFTFNSFAMSWVRQAPGKGLEWVSAISGSGGGTYYA |
| | DSVKGRFTISRDNSKNTLYLQMNSLRAEDTAVYFCAKDKILWFGEPVFDYWGQGTLVTVSS |
| 5 | EIVLTQSPATLSLSPGERATLSCRASQSVSSYLAWYQQKPGQAPRLLIYDASNRATGIPARFSG |
| | SGSGTDFTLTISSLEPEDFAVYYCQQRSNWPPTFGQGTKVEIK |
| 6 | SFAMS |
| 7 | AISGSGGGTYYADSVKG |
| 8 | DKILWFGEPVFDY |
| 9 | RASQSVSSYLA |
| 10 | DASNRAT |
| 11 | QQRSNWPPTF |
| 12 | EVQLLESGGGLVQPGGSLRLSCAVSGFTFNSFAMSWVRQAPGKGLEWVSAISGSGGGTYYA |
| | DSVKGRFTISRDNSKNTLYLQMNSLRAEDTAVYFCAKDKILWFGEPVFDYWGQGTLVTVSS |
| | ASTKGPSVFPLAPSSKSTSGGTAALGCLVKDYFPEPVTVSWNSGALTSGVHTFPAVLQSSGLY |
| | SLSSVVTVPSSSLGTQTYICNVNHKPSNTKVDKRVEPKSCDKTHTCPPCPAPELLGGPSVFLFP |
| | PKPKDTLMISRTPEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREEMTKNQVSLTC |
| | LVKGFYPSDIAVEWESNGQPENNYKTTPPVLDSDGSFFLYSKLTVDKSRWQQGNVFSCSVMH |
| | EALHNHYTQKSLSLSPGK |
| 13 | EIVLTQSPATLSLSPGERATLSCRASQSVSSYLAWYQQKPGQAPRLLIYDASNRATGIPARFSG |
| | SGSGTDFTLTISSLEPEDFAVYYCQQRSNWPPTFGQGTKVEIKRTVAAPSVFIFPPSDEQLKSGT |
| | ASVVCLLNNFYPREAKVQWKVDNALQSGNSQESVTEQDSKDSTYSLSSTLTLSKADYEKHK |
| | VYACEVTHQGLSSPVTKSFNRGEC |
| 14 | QVQLVQSGAEVKKPGSSVKVSCKASGGTFSSYAFSWVRQAPGQGLEWMGRVIPFLGIANSA |
| | QKFQGRVTITADKSTSTAYMDLSSLRSEDTAVYYCARDDIAALGPFDYWGQGTLVTVSSAS |
| 15 | DIQMTQSPSSLSASVGDRVTITCRASQGISSWLAWYQQKPEKAPKSLIYAASSLQSGVPSRFSG |
| | SGSGTDFTLTISSLQPEDFATYYCQQYNSYPRTFGQGTKVEIK |
| 16 | EVQLVQSGAEVKKPGESLKISCKGSGYSFSNYWIGWVRQMPGKGLEWMGIIYPHDSDARYSP |
| | SFQGQVTFSADKSISTAYLQWSSLKASDTAMYYCARHVGWGSRYWYFDLWGRGTLVTVSS |
| 17 | EIVLTQSPATLSLSPGERATLSCRASQSVSSYLAWYQQKPGQAPGLLIYDASNRASGIPARFSG |
| | SGSGTDFTLTISSLEPEDFAVYYCQQRSNWPLTFGGGTKVEIK |
| 18 | QVQLVESGGGLVQPGGSLRLSCAASGFTFSSYYMNWVRQAPGKGLEWVSGISGDPSNTYYA |
| | DSVKGRFTISRDNSKNTLYLQMNSLRAEDTAVYYCARDLPLVYTGFAYWGQGTLVTVSS |
| 19 | DIELTQPPSVSVAPGQTARISCSGDNLRHYYVYWYQQKPGQAPVLVIYGDSKRPSGIPERFSGS |
| | NSGNTATLTISGTQAEDEADYYCQTYTGGASLVFGGGTKLTVLGQ |
| 20 | QVQLVQSGAEVAKPGTSVKLSCKASGYTFTDYWMQWVKQRPGQGLEWIGTIYPGDGDTGY |
| | AQKFQGKATLTADKSSKTVYMHLSSLASEDSAVYYCARGDYYGSNSLDYWGQGTSVTVSS |
| 21 | DIVMTQSHLSMSTSLGDPVSITCKASQDVSTVVAWYQQKPGQSPRRLIYSASYRYIGVPDRFT |
| | GSGAGTDFTFTISSVQAEDLAVYYCQQHYSPPYTFGGGTKLEIK |
| 22 | LNFRAPPVIPNVPFLWAWNAPSEFCLGKFDEPLDMSLFSFIGSPRINATGQGVTIFYVDRLGYY |
| | PYIDSITGVTVNGGIPQKISLQDHLDKAKKDITFYMPVDNLGMAVIDWEEWRPTWARNWKP |
| | KDVYKNRSIELVQQQNVQLSLTEATEKAKQEFEKAGKDFLVETIKLGKLLRPNHLWGYYLFP |
| | DCYNHHYKKPGYNGSCFNVEIKRNDDLSWLWNESTALYPSIYLNTQQSPVAATLYVRNRVR |
| | EAIRVSKIPDAKSPLPVFAYTRIVFTDQVLKFLSQDELVYTFGETVALGASGIVIWGTLSIMRS |
| | MKSCLLLDNYMETILNPYIINVTLAAKMCSQVLCQEQGVCIRKNWNSSDYLHLNPDNFAIQL |
| | EKGGKFTVRGKPTLEDLEQFSEKFYCSCYSTLSCKEKADVKDTDAVDVCIADGVCIDAFLKPP |
| | METEEPQIFY |
| 23 | QVQLVQSGVEVKKPGASVKVSCKASGYTFTNYYMYWVRQAPGQGLEWMGGINPSNGGTNF |
| | NEKFKNRVTLTTDSSTTTAYMELKSLQFDDTAVYYCARRDYRFDMGFDYWGQGTTVTVSS |
| 24 | EIVLTQSPATLSLSPGERATLSCRASKGVSTSGYSYLHWYQQKPGQAPRLLIYLASYLESGVPA |
| | RFSGSGSGTDFTLTISSLEPEDFAVYYCQHSRDLPLTFGGGTKVEIK |
| 25 | QVQLVESGGGVVQPGRSLRLDCKASGITFSNSGMHWVRQAPGKGLEWVAVIWYDGSKRYY |
| | ADSVKGRFTISRDNSKNTLFLQMNSLRAEDTAVYYCATNDDYWGQGTLVTVSS |
| 26 | EIVLTQSPATLSLSPGERATLSCRASQSVSSYLAWYQQKPGQAPRLLIYDASNRATGIPARFSG |
| | SGSGTDFTLTISSLEPEDFAVYYCQQSSNWPRTFGQGTKVEIK |
| 27 | EVQLVESGGGLVQPGGSLRLSCAASGFTFSRYWMSWVRQAPGKGLEWVANIKQDGSEKYY |
| | VDSVKGRFTISRDNAKNSLYLQMNSLRAEDTAVYYCAREGGWFGELAFDYWGQGTLVTVSS |
| 28 | EIVLTQSPGTLSLSPGERATLSCRASQRVSSSYLAWYQQKPGQAPRLLIYDASSRATGIPDRFS |
| | GSGSGTDFTLTISRLEPEDFAVYYCQQYGSLPWTFGQGTKVEIK |
| 29 | EVQLVESGGGLVQPGGSLRLSCAASGFTFSDSWIHWVRQAPGKGLEWVAWISPYGGSTYYA |
| | DSVKGRFTISADTSKNTAYLQMNSLRAEDTAVYYCARRHWPGGFDYWGQGTLVTVSS |
| 30 | DIQMTQSPSSLSASVGDRVTITCRASQDVSTAVAWYQQKPGKAPKLLIYSASFLYSGVPSRFS |
| | GSGSGTDFTLTISSLQPEDFATYYCQQYLYHPATFGQGTKVEIK |
| 31 | EVQLLESGGGLVQPGGSLRLSCAASGFTFSSYIMMWVRQAPGKGLEWVSSIYPSGGITFYADT |
| | VKGRFTISRDNSKNTLYLQMNSLRAEDTAVYYCARIKLGTVTTVDYWGQGTLVTVSS |
| 32 | QSALTQPASVSGSPGQSITISCTGTSSDVGGYNYVSWYQQHPGKAPKLMIYDVSNRPSGVSNR |
| | FSGSKSGNTASLTISGLQAEDEADYYCSSYTSSSTRVFGTGTKVTVL |
| 33 | QVQLVQSGAEVKKPGSSVKVSCKASGGTFSSYAISWVRQAPGQGLEWMGGIIPIFDTANYAQ |
| | KFQGRVTITADESTSTAYMELSSLRSEDTAVYYCARPGLAAAYDTGSLDYWGQGTLVTVSS |
| 34 | EIVLTQSPATLSLSPGERATLSCRASQSVRSYLAWYQQKPGQAPRLLIYDASNRATGIPARFSG |
| | SGSGTDFTLTISSLEPEDFAVYYCQQRNYWPLTFGQGTKVEIK |
| 35 | EVQLVESGGGLVQPGGSLRLSCAASGFAFSRYDMSWVRQAPGKGLESVAYISGGGANTYYL |
| | DNVKGRFTISRDNAKNSLYLQMNSLRAEDTAVYYCASPYLSYFDVWGQGTLVTVSS |
| 36 | EIVMTQSPATLSVSPGERATLSCRASQSLSDYLHWYQQKPGQAPRLLIKSASQSISGIPARFSGS |
| | GSGTEFTLTISSLQSEDFAVYYCQNGHSFPYTFGQGTKLEIK |
| 37 | EVQLLESGGGLVQPGGSLRLSCAASGFTFSSYAMSWVRQAPGKGLEWVSAISGSGGSTYYAD |
| | SVKGRFTISRDNSKNTLYLQMNSLRAEDTAVYYCAKSPYAPLDYWGQGTLVTVSS |
| 38 | EIVLTQSPATLSLSPGERATLSCRASQSVNDYLAWYQQKPGQAPRLLIYDASNRATGIPARFSG |
| | SGSGTDFTLTISSLEPEDFAVYYCQQGGHAPITFGQGTKVEIK |
| 39 | EVQLVQSGAEVKKPGESLKISCKGSGYSFTSYWMQWVRQMPGKGLEWMGAIYPGDGDIRY |
| | TQNFKGQVTISADKSISTAYLQWSSLKASDTAMYYCARWEKSTTVVQRNYFDYWGQGTTVT |
| | VSS |
| 40 | DIQMTQSPSSLSASVGDRVTITCKASENVGTFVSWYQQKPGKAPKLLIYGASNRYTGVPSRFS |
| | GSGSGTDFTLTISSLQPEDFATYYCGQSYSYPTFGQGTKLEIK |

In some embodiments, the anti-CD38 antibody comprises a VH amino acid sequence of SEQ ID NO: 4 or a VL amino acid sequence of SEQ ID NO: 5, or both. In some embodiments, the anti-CD38 antibody comprises a VH amino acid sequence of SEQ ID NO: 4 and a VL amino acid sequence of SEQ ID NO: 5. In some embodiments, the anti-CD38 antibody comprises a VH amino acid sequence that is at least 95% identical to SEQ ID NO: 4 and a VL amino acid sequence that is at least 95% identical to SEQ ID NO: 5.

In some embodiments, the anti-CD38 antibody comprises:
a) a heavy chain complementarity determining region 1 (HCDR1), HCDR2 and HCDR3 amino acid sequences of SEQ ID NOs: 6, 7 and 8, respectively; or
b) a light chain complementarity determining region 1 (LCDR1), LCDR2 and LCDR3 amino acid sequences of SEQ ID NOs: 9, 10 and 11, respectively,
c) or both a) and b).

In some embodiments, the anti-CD38 antibody comprises:
a) a HCDR1, HCDR2 and HCDR3 amino acid sequences of SEQ ID NOs: 6, 7 and 8, respectively; and
b) a LCDR1, LCDR2 and LCDR3 amino acid sequences of SEQ ID NOs: 9, 10 and 11, respectively.

In some embodiments, the anti-CD38 antibody comprises a heavy chain amino acid sequence of SEQ ID NO: 12. In some embodiments, the anti-CD38 antibody comprises a heavy chain amino acid sequence that is at least 90% identical, e.g., about: 90%, 91%, 92%, 93%, 94%, 95%, 96%, 97%, 98% or 99% identical to SEQ ID NO: 12.

In some embodiments, the anti-CD38 antibody comprises a light chain amino acid sequence of SEQ ID NO: 13. In some embodiments, the anti-CD38 antibody comprises a light chain amino acid sequence that is at least 90% identical, e.g., about: 90%, 91%, 92%, 93%, 94%, 95%, 96%, 97%, 98% or 99% identical to SEQ ID NO: 13.

In some embodiments, the anti-CD38 antibody comprises a heavy chain amino acid sequence of SEQ ID NO: 12 or a light chain amino acid sequence of SEQ ID NO: 13, or both. In some embodiments, the anti-CD38 antibody comprises a heavy chain amino acid sequence of SEQ ID NO: 12 and a light chain amino acid sequence of SEQ ID NO: 13. In some embodiments, the anti-CD38 antibody comprises a heavy chain amino acid sequence that is at least 95% identical to SEQ ID NO: 12 and a light chain amino acid sequence that is at least 95% identical to SEQ ID NO: 13.

In some embodiments, the anti-CD38 antibody is of IgG1, IgG2, IgG3 or IgG4 subtype. In some embodiments, the anti-CD38 antibody is of IgG1 subtype. In some embodiments, the anti-CD38 antibody is of κ subtype. In some embodiments, the anti-CD38 antibody is of IgG1/κ subtype.

In some embodiments, the anti-CD38 antibody is daratumumab. Daratumumab is of IgG1/κ subtype and is described in U.S. Pat. No. 7,829,673. Daratumumab comprises a HCDR1, HCDR2 and HCDR3 amino acid sequences of SEQ ID NOs: 6, 7 and 8, respectively; and a LCDR1, LCDR2 and LCDR3 amino acid sequences of SEQ ID NOs: 9, 10 and 11, respectively. Daratumumab comprises a VH amino acid sequence of SEQ ID NO: 4, and a VL amino acid sequence of SEQ ID NO: 5. Daratumumab comprises a heavy chain amino acid sequence of SEQ ID NO: 12, and a light chain amino acid sequence of SEQ ID NO: 13.

In some embodiments, the anti-CD38 antibody comprises a mutation in at least one amino acid residue selected from those corresponding to E345, E430, 5440, Q386, P247, 1253, S254, Q311, D/E356, T359, E382, Y436, and K447 in the Fc-region of a human IgG1 heavy chain, to increase an effector function. Non-limiting examples of the effector functions include antibody-dependent cell-mediated cytotoxicity (ADCC), antibody-dependent cellular phagocytosis (ADCP), binding to complement receptor of an opsonized antibody mediated by the antibody, C1q-binding, complement activation, complement-dependent cellular cytotoxicity (CDCC), complement-dependent cytotoxicity (CDC), complement-enhanced cytotoxicity, downmodulation, Fc-gamma receptor-binding, FcRn-binding, induction of apoptosis, internalization, oligomer (e.g., hexamer) formation, oligomer (e.g., hexamer) stability, opsonization, Protein A-binding and Protein G-binding. Non-limiting examples of mutations, e.g., ones that increases hexamer formation, hexamer stability or both can be found in Int. Pat. Publ. Nos. WO 13/004842 and WO 20/012036, incorporated by reference in their entirety. In some embodiments, the anti-CD38 antibody is HexaBody-CD38 (GEN3014).

Other non-limiting examples of anti-CD38 antibodies that may be used in the methods of the invention include mAb003, mAb024, MOR-202 (MOR-03087), Isatuximab, and anti-CD38 antibodies described in Int. Pat. Publ. Nos. WO05/103083, WO06/125640, WO07/042309, WO08/047242 and WO14/178820, etc. MAb003, comprising the VH and the VL amino acid sequences of SEQ ID NOs: 14 and 15, respectively, is described in U.S. Pat. No. 7,829,673. MAb024, comprising the VH and the VL amino acid sequences of SEQ ID NOs: 16 and 17, respectively, is described in U.S. Pat. No. 7,829,673. MOR-202 (MOR-03087), comprising the VH and the VL amino acid sequences of SEQ ID NOs: 18 and 19, respectively, is described in U.S. Pat. No. 8,088,896. Isatuximab, comprising the VH and the VL amino acid sequences of SEQ ID NOs: 20 and 21, respectively, is described in U.S. Pat. No. 8,153,765. The VH and the VL of mAb003, mAb024, MOR-202 or Isatuximab, or a combination thereof, may be expressed as IgG1/κ.

In some embodiments, the anti-CD38 antibody comprises the HCDR1, HCDR2, HCDR3, LCDR1, LCDR2, and LCDR3 amino acid sequences of:
a) the VH of SEQ ID NO: 14 and the VL of SEQ ID NO: 15;
b) the VH of SEQ ID NO: 16 and the VL of SEQ ID NO: 17;
c) the VH of SEQ ID NO: 18 and the VL of SEQ ID NO: 19; or
d) the VH of SEQ ID NO: 20 and the VL of SEQ ID NO: 21.

In some embodiments, the anti-CD38 antibody comprises the VH and VL amino acid sequences of:
a) SEQ ID NOs: 14 and 15, respectively;
b) SEQ ID NOs: 16 and 17, respectively;
c) SEQ ID NOs: 18 and 19, respectively; or
d) SEQ ID NOs: 20 and 21, respectively.

In some embodiments, the anti-CD38 antibody is HexaBody-CD38 (GEN3014).

Anti-CD38 antibodies used in the methods of the invention may also be selected de novo from, e.g., a phage display library, where the phage is engineered to express human immunoglobulins or portions thereof such as Fabs, single chain antibodies (scFv), or unpaired or paired antibody variable regions (Knappik et al., J. Mol. Biol. 296:57-86 (2000); Krebs et al., J. Immunol. Meth. 254:67-84 (2001); Vaughan et al., Nature Biotechnology 14:309-14 (1996); Sheets et al., PITAS (USA) 95:6157-62 (1998); Hoogenboom & Winter, J. Mol. Biol. 227:381 (1991); Marks et al., J. Mol. Biol. 222:581 (1991)). CD38 binding variable domains may be isolated from e.g., phage display libraries expressing antibody heavy and light chain variable regions as fusion proteins with bacteriophage pIX coat protein as described in Shi et al., J. Mol. Biol. 397:385-96 (2010) and Intl. Pat. Publ. No. WO09/085462. The antibody libraries may be screened for binding to human CD38 extracellular domain; obtained positive clones further characterized; Fabs isolated from the clone lysates, and subsequently cloned as full-length antibodies. Such phage display methods for isolating human antibodies are established in the art. See for example: U.S. Pat. Nos. 5,223,409, 5,403,484, 5,427,908, 5,571,698, 5,580,717, 5,885,793, 5,969,108, 6,172,197, 6,521,404, 6,544,731, 6,555,313, 6,582,915 and 6,593,081.

In some embodiments, the anti-CD38 antibody binds human CD38 with a dissociation constant (K_{D}) of less than about: 1×10⁻⁷ M, 1×10⁻⁸ M, 1×10⁻⁹ M, 1×10⁻¹⁰M, 1×10⁻¹¹ M, 1×10⁻¹² M, 1×10⁻¹³ M, 1×10⁻¹⁴ M or 1×10⁻¹⁵ M, as determined by surface plasmon resonance or the KinExA method, as practiced by those of skill in the art. In some embodiments, the antibody binds human CD38 with a K_{D} of less than about 1×10⁻⁸ M. In some embodiments, the antibody binds human CD38 with a K_{D} of less than about 1×10⁻⁹ M.

KinExA instrumentation, ELISA or competitive binding assays are known to those skilled in the art. The measured affinity of a particular antibody/CD38 interaction may vary if measured under different conditions (e.g., osmolarity, pH). Thus, measurements of affinity and other binding parameters (e.g., K_{D}, Kₒₙ, K_{off}) are typically made with standardized conditions and a standardized buffer. Those skilled in the art will appreciate that the internal error for affinity measurements, for example, using Biacore 3000 or ProteOn (measured as standard deviation, SD) may typically be within 5-33% for measurements within the typical limits of detection. Therefore, the term “about” in the context of K_{D} reflects the typical standard deviation in the assay. For example, the typical SD for a K_{D} of 1×10⁻⁹ M is up to ±0.33×10⁻⁹ M.

The term “antibodies” is meant in a broad sense and includes immunoglobulin molecules including full length antibodies, antigen-binding fragments, monospecific and multispecific (e.g., bispecific) antibodies, monoclonal antibodies (including murine, human, humanized and chimeric antibodies), dimeric, tetrameric or multimeric antibodies, single chain antibodies, domain antibodies and any other modified configuration of the immunoglobulin molecule that comprises an antigen binding site of the required specificity.

“Full length antibodies” comprise two heavy (H) chains and two light (L) chains inter-connected by disulfide bonds as well as multimers thereof (e.g., IgM). Each heavy chain comprises a heavy chain variable region (VH) and a heavy chain constant region (comprising domains CH1, hinge, CH2 and CH3). Each light chain comprises a light chain variable region (VL) and a light chain constant region (CL). The VH and the VL regions may be further subdivided into regions of hypervariability, termed complementarity determining regions (CDRs), interspersed with framework regions (FRs). Each VH and VL composes three CDRs and four FRs, arranged from amino-terminus to carboxy-terminus in the following order: FR1, CDR1, FR2, CDR2, FR3, CDR3, and FR4.

“Complementarity determining regions (CDRs)” are “antigen binding sites” in an antibody. CDRs may be defined using various terms: (i) HCDR1, HCDR2, HCDR3, LCDR1, LCDR2 and LCDR3, based on sequence variability (Wu and Kabat, J. Exp. Med. 132:211-50 (1970); Kabat et al., Sequences of Proteins of Immunological Interest, 5th Ed. Public Health Service, National Institutes of Health, Bethesda, Md. (1991)); (ii) “Hypervariable regions” (HVR or HV) H1, H2, H3, L1, L2 and L3, based on structure as defined by Chothia and Lesk (Chothia & Lesk, Mol. Biol. 196:901-17 (1987)); (iii) the International ImMunoGeneTics (IMGT) database (www_imgt_org) provides a standardized numbering and definition of antigen-binding sites. The correspondence between CDRs, HVs and IMGT delineations is described in Lefranc et al., Dev. Comparat. Immunol. 27:55-77 (2003). The term “CDR”, “HCDR1”, “HCDR2”, “HCDR3”, “LCDR1”, “LCDR2” and “LCDR3” as used herein includes CDRs defined by any of the methods described supra, in Kabat, Chothia and Lesk, or IMGT, unless explicitly stated otherwise.

Immunoglobulins may be assigned to five major classes: IgA, IgD, IgE, IgG and IgM, depending on the heavy chain constant domain amino acid sequence. IgA is further sub-classified as the isotypes IgA₁, IgA₂. IgG is further sub-classified as IgG₁, IgG₂, IgG₃ and IgG₄. Antibody light chains of any vertebrate species can be assigned to one of two clearly distinct types, namely kappa (κ) and lambda (λ), based on the amino acid sequences of their constant domains.

“Antigen-binding fragment” refers to a portion of an immunoglobulin molecule that retains the antigen binding properties of the parental full-length antibody. Non-limiting examples of antigen-binding fragments include heavy chain complementarity determining regions (HCDR) 1, 2 and/or 3, light chain complementarity determining regions (LCDR) 1, 2 and/or 3, a heavy chain variable region (VH), or a light chain variable region (VL), Fab, F(ab′)₂, Fd and Fv fragments, as well as domain antibodies (dAb) consisting of either one VH domain or one VL domain. VH and VL domains may be linked together via a synthetic linker to form various types of single chain antibody designs in which the VH/VL domains pair intramolecularly, or intermolecularly in those cases when the VH and VL domains are expressed by separate chains, to form a monovalent antigen binding site, such as single chain Fv (scFv) or diabody. See, for example, Int. Pat. Publ. Nos. WO1998/44001, WO1988/01649, WO1994/13804 and WO1992/01047.

“Monoclonal antibody” refers to an antibody population with single amino acid composition in each heavy and each light chain, except for possible well-known alterations such as removal of C-terminal lysine from the antibody heavy chain. Monoclonal antibodies may have heterogeneous glycosylation within the antibody population. A monoclonal antibody may be monovalent, bivalent or multivalent.

A monoclonal antibody may be monospecific or multispecific (e.g., bispecific). Monospecific antibodies bind one antigenic epitope.

“Multispecific” refers to an antibody that specifically binds at least two distinct antigens or at least two distinct epitopes within the antigens, for example three, four or five distinct antigens or epitopes.

“Bispecific” refers to an antibody that specifically binds two distinct antigens or two distinct epitopes within the same antigen.

“Isolated antibody” refers to an antibody or an antigen-binding fragment thereof that is substantially free of other antibodies having different antigenic specificities (e.g., an isolated anti-CD38 antibody is substantially free of antibodies that specifically bind antigens other than human CD38). In the case of a bispecific antibody, the bispecific antibody specifically binds two antigens of interest, and is substantially free of antibodies that specifically bind antigens other than the two antigens of interest. In some embodiments, the anti-CD38 antibody is at least 80% pure, e.g., about: 80%, 81%, 82%, 83%, 84%, 85%, 86%, 87%, 88%, 89%, 90%, 91%, 92%, 93%, 94%, 95%, 96%, 97%, 98%, 99% or 100% pure.

In some embodiments, the anti-CD38 antibody is a humanized antibody or a human antibody. In some embodiments, the anti-CD38 antibody is a human antibody.

“Humanized antibodies” refers to antibodies in which the antigen binding sites are derived from non-human species and the variable region frameworks are derived from human immunoglobulin sequences. Humanized antibodies may include intentionally introduced mutations in the framework regions so that the framework may not be an exact copy of expressed human immunoglobulin or germline gene sequences.

“Human antibodies” refers to antibodies having heavy and light chain variable regions in which both the framework and the antigen binding site are derived from sequences of human origin. If the antibody contains a constant region or a portion of the constant region, the constant region is also derived from sequences of human origin. Antibodies in which antigen binding sites are derived from a non-human species are not included in the definition of “human antibody.”

A human antibody comprises heavy or light chain variable regions that are derived from sequences of human origin if the variable regions of the antibody are obtained from a system that uses human germline immunoglobulin or rearranged immunoglobulin genes. Non-limiting example systems include human immunoglobulin gene libraries displayed on phage, and transgenic non-human animals such as mice or rats carrying human immunoglobulin loci. A human antibody typically contains amino acid differences when compared to the human germline or rearranged immunoglobulin sequences due to, for example, naturally occurring somatic mutations, intentional substitutions in the framework or antigen binding site, and substitutions introduced during cloning or VDJ recombination in non-human animals. Typically, a human antibody is at least 80% identical in amino acid sequence to an amino acid sequence encoded by a human germline or rearranged immunoglobulin gene. For example, about: 80%, 81%, 82%, 83%, 84%, 85%, 86%, 87%, 88%, 89%, 90%, 91%, 92%, 93%, 94%, 95%, 96%, 97%, 98%, 99% or 100% identical. In some cases, a human antibody may contain consensus framework sequences derived from human framework sequence analyses (see, e.g., Knappik et al., J. Mol. Biol. 296:57-86 (2000)), or synthetic HCDR3 incorporated into human immune-globulin gene libraries displayed on phage (see, e.g., Shi et al., J. Mol. Biol. 397:385-96 (2010) and Int. Pat. Publ. No. WO2009/085462).

“Recombinant” includes antibodies and other proteins that are prepared, expressed, created or isolated by recombinant means.

“Epitope” refers to a portion of an antigen to which an antibody specifically binds. Epitopes typically consist of chemically active (such as polar, non-polar or hydrophobic) surface groupings of moieties such as amino acids or polysaccharide side chains and may have specific three-dimensional structural characteristics, as well as specific charge characteristics. An epitope may be composed of contiguous and/or discontiguous amino acids that form a conformational spatial unit. For a discontiguous epitope, amino acids from differing portions of the linear sequence of the antigen come into close proximity in a three-dimensional space through the folding of the protein molecule.

“Variant” refers to a polypeptide or a polynucleotide that differs from a reference polypeptide or a reference polynucleotide by one or more modifications, for example, substitutions, insertions, deletions or a combination thereof.

### Administration/Pharmaceutical Compositions

In the methods of the invention, the anti-CD38 antibody may be provided in a suitable pharmaceutical composition comprising the anti-CD38 antibody and a pharmaceutically acceptable carrier.

“Pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative. The carrier may be diluent, adjuvant, excipient, or vehicle with which the anti-CD38 antibody is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the anti-CD38 antibody in such pharmaceutical formulation may vary widely, i.e., from less than about 0.5%, to at least about 1%, or to as much as 15% or 20%, 25%, 30%, 35%, 40%, 45% or 50% by weight. The concentration will be selected primarily based on required dose, fluid volumes, viscosities, etc., according to the mode of administration. Suitable vehicles and formulations, inclusive of other human proteins, e.g., human serum albumin, are described, for example, in Remington: The Science and Practice of Pharmacy, 21^{st} Edition, Troy, D. B. ed., Lipincott Williams and Wilkins, Philadelphia, Pa. 2006, Part 5, Pharmaceutical Manufacturing: 691-1092 (e.g., pages 958-89).

The mode of administration of the anti-CD38 antibody may be any suitable parenteral administration. Non-limiting examples of administration include intradermal, intramuscular, intraperitoneal, intravenous, subcutaneous, pulmonary, transmucosal (oral, intranasal, intravaginal, rectal), etc.

In some embodiments, the anti-CD38 antibody is administered by intravenous infusion. In some embodiments, the intravenous infusion is given over 15, 30, 45 or 60 minutes. In some embodiments, the intravenous infusion is given over 1.5, 2, 3, 4, 5, 6, 7, 8, 9, 10, 11 or 12 hours.

The dose of the anti-CD38 antibody given to a patient is sufficient to alleviate or at least partially arrest the disease being treated (“therapeutically effective amount”). Non-limiting examples of therapeutically effective amounts include about 0.005 mg to about 100 mg/kg, e.g. about: 0.05-30, 5-25, 1, 2, 3, 4, 5, 6, 7, 8, 9, 10, 15, 16, 17, 18, 19, 20, 21, 22, 23, 24, 25, 30, 40, 50, 60, 70, 80, 90 or 100 mg/kg.

A fixed unit dose may also be given, for example, 50, 100, 200, 500 or 1000 mg. In some embodiments, the dose is based on the patient's surface area, e.g., 500, 400, 300, 250, 200, or 100 mg/m². The dosage may also depend on the disease. Usually between 1 and 8 doses, e.g., 1, 2, 3, 4, 5, 6, 7 or 8, may be administered to treat AL. In some embodiments, 9, 10, 11, 12, 13, 14, 15, 16, 17, 18, 19, 20 or more doses may be administered.

The administration of the anti-CD38 antibody may be repeated. For example, after 1, 2, 3, 4, 5 or 6 days, 1, 2, 3, 4, 5, 6 or 7 weeks, or 1, 2, 3, 4, 5 or 6 months, or longer. Repeated courses of treatment are also possible, as is chronic administration. The repeated administration may be at the same dose or at a different dose. For example, the anti-CD38 antibody may be administered at 8 mg/kg or at 16 mg/kg at weekly interval for 8 weeks, followed by administration at 8 mg/kg or at 16 mg/kg every two weeks for an additional 16 weeks, followed by administration at 8 mg/kg or at 16 mg/kg every four weeks by intravenous infusion.

In some embodiments, the anti-CD38 antibody is administered at 16 mg/kg once a week for 8 weeks, followed by administration at 16 mg/kg once every two weeks for 16 weeks, followed by administration at 16 mg/kg once every four weeks until discontinuation.

In some embodiments, the anti-CD38 antibody is administered at 8 mg/kg once a week for 8 weeks, followed by administration at 8 mg/kg once every two weeks for 16 weeks, followed by administration at 8 mg/kg once every four weeks until discontinuation.

In some embodiments, the anti-CD38 antibody is administered at 16 mg/kg once a week for 4 weeks, followed by administration at 16 mg/kg once every two weeks for 16 weeks, followed by administration at 16 mg/kg once every four weeks until discontinuation.

In some embodiments, the anti-CD38 antibody is administered at 8 mg/kg once a week for 4 weeks, followed by administration at 8 mg/kg once every two weeks for 16 weeks, followed by administration at 8 mg/kg once every four weeks until discontinuation.

The anti-CD38 antibody may be administered as maintenance therapy, such as, e.g., once a week for a period of 6 months or more.

For example, the anti-CD38 antibody may be provided as a daily dosage in an amount of about 0.1-100 mg/kg, such as about 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

Daratumumab is indicated for the treatment of adult patients with multiple myeloma. For example, in combination with lenalidomide and dexamethasone in newly diagnosed patients who are ineligible for autologous stem cell transplant and in patients with relapsed or refractory multiple myeloma who have received at least one prior therapy; in combination with bortezomib, melphalan and prednisone in newly diagnosed patients who are ineligible for autologous stem cell transplant; in combination with bortezomib, thalidomide, and dexamethasone in newly diagnosed patients who are eligible for autologous stem cell transplant; in combination with bortezomib and dexamethasone in patients who have received at least one prior therapy; in combination with carfilzomib and dexamethasone in patients who have received one to three prior lines of therapy; in combination with pomalidomide and dexamethasone in patients who have received at least two prior therapies including lenalidomide and a proteasome inhibitor; or as monotherapy, in patients who have received at least three prior lines of therapy including a proteasome inhibitor (PI) and an immunomodulatory agent or who are double-refractory to a PI and an immunomodulatory agent. Additional information regarding daratumumab can be found, for example, in the prescribing information product insert for DARZALEX® (wwwjanssenlabels.com/package-insert/product-monograph/prescribing-information/DARZALEX-pi.pdf), which is incorporated herein by reference.

The anti-CD38 antibody may also be administered prophylactically to reduce the risk of developing cancer, delay the onset of the occurrence of an event in cancer progression, and/or reduce the risk of recurrence when a cancer is in remission. This may be especially useful in patients wherein it is difficult to locate a tumor that is known to be present due to other biological factors.

The anti-CD38 antibody may be lyophilized for storage and reconstituted in a suitable carrier prior to use. This technique has been shown to be effective with conventional protein preparations and well known lyophilization and reconstitution techniques can be employed.

In some embodiments, the anti-CD38 antibody is administered intravenously.

In some embodiments, the anti-CD38 antibody is administered subcutaneously.

In some embodiments, the anti-CD38 antibody is administered subcutaneously in a pharmaceutical composition comprising the anti-CD38 antibody and a hyaluronidase. In some embodiments, the hyaluronidase is rHuPH20 recombinant hyaluronidase. In some embodiments, the hyaluronidase is rHuPH20 having the amino acid sequence of SEQ ID NO: 22.

Hyaluronidase is an enzyme that degrades hyaluronic acid (EC 3.2.1.35) and lowers the viscosity of hyaluronan in the extracellular matrix, thereby increasing tissue permeability. rHuPH20 is a recombinant hyaluronidase (HYLENEX® recombinant) and is described in Int. Pat. Publ. No. WO2004/078140.

Additional information regarding daratumumab and hyaluronidase can be found, for example, in the prescribing information product insert for DARZALEX FASPRO™ (www.janssenlabels.com/package-insert/product-monograph/prescribing-information/DARZALEX+Faspro-pi.pdf), which is incorporated herein by reference.

The administration of the pharmaceutical composition comprising the anti-CD38 antibody and the hyaluronidase may be repeated after one day, two days, three days, four days, five days, six days, one week, two weeks, three weeks, four weeks, five weeks, six weeks, seven weeks, two months, three months, four months, five months, six months or longer. Repeated courses of treatment are also possible, as is chronic administration. The repeated administration may be at the same dose or at a different dose. For example, the pharmaceutical composition comprising the anti-CD38 antibody and the hyaluronidase may be administered once weekly for eight weeks, followed by once in two weeks for 16 weeks, followed by once in four weeks. The pharmaceutical compositions to be administered may comprise about 1,800 mg of the anti-CD38 antibody and about 30,000 U of hyaluronidase. In some embodiments, the concentration of the anti-CD38 antibody in the pharmaceutical composition is about 120 mg/ml. The pharmaceutical composition comprising the anti-CD38 antibody and the hyaluronidase may be administered subcutaneously to the abdominal region. The pharmaceutical composition comprising the anti-CD38 antibody and the hyaluronidase may be administered in a total volume of about 15 ml.

In some embodiments, pharmaceutical composition comprising the anti-CD38 antibody and the hyaluronidase is a fixed combination. “Fixed combination” refers to a single pharmaceutical composition comprising two or more compounds, for example, the anti-CD38 antibody and the hyaluronidase administered simultaneously in the form of a single entity or dosage.

In some embodiments, pharmaceutical composition comprising the anti-CD38 antibody and the hyaluronidase is a non-fixed combination. “Non-fixed combination” refers to separate pharmaceutical compositions, wherein each comprises one or more compounds, for example, the anti-CD38 antibody and the hyaluronidase or unit dosage forms administered as separate entities either simultaneously, concurrently or sequentially with no specific intervening time limits, wherein such administration provides effective levels of the two compounds in the body of the subject.

“Treat” or “treatment” refers to therapeutic treatment wherein the object is to slow down (lessen) an undesired physiological change or disease, such as the development or spread of tumor or tumor cells, or to provide a beneficial or desired clinical outcome during treatment. Beneficial or desired clinical outcomes include alleviation of symptoms, diminishment of extent of disease, stabilized (i.e., not worsening) state of disease, delay or slowing of disease progression, lack of metastasis, amelioration or palliation of the disease state, and remission (whether partial or total), whether detectable or undetectable. “Treatment” may also mean prolonging survival as compared to expected survival if a subject was not receiving treatment. Those in need of treatment include those subjects already with the undesired physiological change or disease well as those subjects prone to have the physiological change or disease.

“Therapeutically effective amount” refers to an amount effective, at dosages and for periods of time necessary, to achieve the desired therapeutic result. A therapeutically effective amount may vary according to factors such as the disease state, age, sex, and weight of the individual, and the ability of a therapeutic or a combination of therapeutics to elicit a desired response in the individual. Example indicators of an effective therapeutic or combination of therapeutics include, for example, improved well-being of the patient, reduction in a tumor burden, arrested or slowed growth of a tumor, and/or absence of metastasis of cancer cells to other locations in the body.

“Inhibits growth” (e.g., referring to tumor cells) refers to a measurable decrease in the tumor cell growth or tumor tissue in vitro or in vivo when contacted with a therapeutic or a combination of therapeutics or drugs, when compared to the growth of the same tumor cells or tumor tissue in the absence of the therapeutic or the combination of therapeutic drugs. Inhibition of growth of a tumor cell or tumor tissue in vitro or in vivo may be at least about 10%, 20%, 30%, 40%, 50%, 60%, 70%, 80%, 90%, 99%, or 100%.

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

### Cancer

In some embodiments, the disease is cancer. In some embodiments, the cancer is a CD38-positive cancer. In some embodiments, the cancer is a CD38-negative cancer. In some embodiments, the cancer is a metastatic cancer.

In some embodiments, the cancer is a hematologic cancer.

In some embodiments, the hematologic cancer is leukemia. In some embodiments, the leukemia is acute lymphoblastic leukemia (ALL), acute myeloid leukemia (AML), chronic lymphocytic leukemia (CLL), chronic myeloid leukemia (CIVIL), hairy cell leukemia (HCL) or myelodysplastic syndromes (MDS), or a combination thereof.

In some embodiments, the hematologic cancer is lymphoma.

In some embodiments, the lymphoma is Hodgkin lymphoma. In some embodiments, the Hodgkin lymphoma is nodular sclerosis Hodgkin lymphoma (NSCHL), mixed cellularity Hodgkin lymphoma (MCcHL), lymphocyte-rich Hodgkin's disease (LRCHL) or lymphocyte-depleted Hodgkin's disease (LDHL), or a combination thereof.

In some embodiments, the lymphoma is non-Hodgkin lymphoma (NHL).

In some embodiments, the non-Hodgkin lymphoma is a B cell lymphoma. In some embodiments, the B cell lymphoma is diffuse large B-cell lymphoma (DLBCL), primary mediastinal B cell lymphoma (PMBCL), follicular lymphoma (FL), small lymphocytic lymphoma (SLL), marginal zone lymphoma (MZL), mantle cell lymphoma (MCL), Waldenström's macroglobulinemia (WMG) or Burkitt lymphoma (BL), or a combination thereof.

In some embodiments, the non-Hodgkin lymphoma is a T cell lymphoma. In some embodiments, the T cell lymphoma is peripheral T-cell lymphoma (PTCL), anaplastic large cell lymphoma (ALCL), angioimmunoblastic T-cell lymphoma (AITL) or cutaneous T cell lymphoma, or a combination thereof.

In some embodiments, the hematologic cancer is multiple myeloma. In some embodiments, the multiple myeloma is light chain multiple myeloma (LCMM), non-secretory multiple myeloma (NSMM), solitary plasmacytoma (SP), extramedullary plasmacytoma (EMP), monoclonal gammopathy of undetermined significance (MGUS), smoldering Multiple Myeloma (SMM), Immunoglobulin D multiple myeloma (IgD MM) or Immunoglobulin E (IgE) multiple myeloma, or a combination thereof.

In some embodiments, the hematologic cancer is a CD38-positive hematological malignancy. In some embodiments, the CD38-positive hematological malignancy is multiple myeloma (MM), acute lymphoblastic leukemia (ALL), non-Hodgkin's lymphoma (NHL), diffuse large B-cell lymphoma (DLBCL), Burkitt's lymphoma (BL), follicular lymphoma (FL), mantle-cell lymphoma (MCL), acute myeloid leukemia (AML) or chronic lymphocytic leukemia (CLL), or a combination thereof.

“CD38-positive hematological malignancy” refers to a hematological malignancy characterized by the presence of tumor cells expressing CD38 including leukemias, lymphomas and myeloma. Examples of such CD38-positive hematological malignancies include precursor B-cell lymphoblastic leukemia/lymphoma and B-cell non-Hodgkin's lymphoma, acute promyelocytic leukemia, acute lymphoblastic leukemia and mature B-cell neoplasms, such as B-cell chronic lymphocytic leukemia (CLL)/small lymphocytic lymphoma (SLL), B-cell acute lymphocytic leukemia, B-cell prolymphocytic leukemia, lymphoplasmacytic lymphoma, mantle cell lymphoma (MCL), follicular lymphoma (FL), including low-grade, intermediate-grade and high-grade FL, cutaneous follicle center lymphoma, marginal zone B-cell lymphoma (MALT type, nodal and splenic type), hairy cell leukemia, diffuse large B-cell lymphoma (DLBCL), Burkitt's lymphoma (BL), plasmacytoma, multiple myeloma, plasma cell leukemia, post-transplant lymphoproliferative disorder, light chain amyloidosis, Waldenström's macroglobulinemia, plasma cell leukemias and anaplastic large-cell lymphoma (ALCL).

In some embodiments, the CD38-positive hematological malignancy is a plasma cell disease. In some embodiments, the plasma cell disease is light chain amyloidosis (AL), multiple myeloma (MM) or Waldenström's macroglobulinemia. In some embodiments, the plasma cell disease is MM or AL.

In some embodiments, the disease is MM. In some embodiments, MM is relapsed or refractory MM. In some embodiments, MM is newly diagnosed MM.

In some embodiments, the disease is AL. In some embodiments, AL is cardiac stage I, cardiac stage II or cardiac stage III. In some embodiments, AL is relapsed or refractory AL. In some embodiments, AL is newly diagnosed AL.

In some embodiments, the subject having AL is homozygous for phenylalanine at position 158 of CD16 (FcγRIIIa-158F/F genotype) or heterozygous for valine and phenylalanine at position 158 of CD16 (FcγRIIIa-158F/V genotype). CD16 is also known as the Fc gamma receptor IIIa (FcγRIIIa) or the low affinity immunoglobulin gamma Fc region receptor III-A isoform. Valine/phenylalanine (V/F) polymorphism at FcγRIIIa protein residue at position 158 has been shown to affect FcγRIIIa affinity to human IgG. Receptor with FcγRIIIa-158F/F or FcγRIIIa-158F/V polymorphism has reduced Fc engagement and therefore reduced ADCC when compared to the FcγRIIIa-158V/V. The lack of or low amount of fucose on human N-linked oligosaccharides improves the ability of the antibodies to induce ADCC due to improved binding of the antibodies to human FcγRIIIa (CD16) (Shields et al., J. Biol. Chem. 277: 26733-40 (2002)). Patients can be analyzed for their FcγRIIIa polymorphism using routine methods.

In some embodiments, the anti-CD38 antibody induces in vitro killing of CD38-expressing pathogenic plasma cells by antibody-dependent cell-mediated cytotoxicity (ADCC), antibody-dependent cellular phagocytosis (ADCP), complement dependent cytotoxicity (CDC), apoptosis, or in vitro modulation of CD38 enzymatic activity, wherein the subject is homozygous for valine at position 158 of CD16.

In some embodiments, the cancer is a solid tumor.

In some embodiments, the solid tumor is a tumor of the breast, lung, prostate, colon, bladder, ovary, kidney, stomach, colon, rectum, testes, head and/or neck, pancreas, brain, skin, or a combination thereof.

In some embodiments, the solid tumor is bladder cancer, brain cancer, breast cancer, cervical cancer, colon cancer, colorectal cancer, fallopian tube cancer, gastric cancer, genitourinary cancer, head and neck cancer, liver cancer, lung cancer, melanoma, nasopharyngeal carcinoma, pancreatic cancer, prostate cancer, ovarian cancer, rectal cancer, renal cancer, skin cancer, stomach cancer, testicular cancer, thyroid cancer or urethral cancer, or a combination thereof.

In some embodiments, the solid tumor is squamous non-small cell lung cancer (NSCLC), non-squamous NSCLC, lung adenocarcinoma, mesothelioma, kidney clear cell carcinoma, kidney papillary cell carcinoma, castration-resistant prostate cancer, squamous cell carcinoma of the head and neck, carcinomas of the esophagus, carcinomas of the gastrointestinal tract or endometriosis, or a combination thereof.

In some embodiments, the solid tumor is a melanoma, a lung cancer, a squamous non-small cell lung cancer (NSCLC), a non-squamous NSCLC, a colorectal cancer, a prostate cancer, a castration-resistant prostate cancer, a stomach cancer, an ovarian cancer, a gastric cancer, a liver cancer, a pancreatic cancer, a thyroid cancer, a squamous cell carcinoma of the head and neck, a carcinoma of the esophagus or gastrointestinal tract, a breast cancer, a fallopian tube cancer, a brain cancer, an urethral cancer, a genitourinary cancer, an endometriosis, a cervical cancer or a metastatic lesion of the cancer.

In some embodiments, the solid tumor is a CD38-positive solid tumor. In some embodiments, the solid tumor is a CD38-negative solid tumor.

In some embodiments, the solid tumor is a metastatic lesion of the cancer.

In some embodiments, the disease is a MDSC related disease. “MDSC related disease” refers to a disease or disorder linked to myeloid-derived suppressor cells (MDSCs). MDSC related disease may be caused by a MDSC function, for example, suppression of an anti-tumor response or effector T cell proliferation. The MDSC mediated disease may be cancer. “MDSC related disease” and “MDSC mediated disease” are used exchangeably herein.

In some embodiments, the disease is a Breg related disease. “Breg related disease” refers to a disease or disorder linked to regulatory B cells. Breg related disease may be caused by for example Breg mediated suppression of an antitumor response or effector T cell proliferation. The Breg mediated disease may be cancer. “Breg related disease” and “Breg mediated disease” are used exchangeably herein.

### Neurological Disorders

In some embodiments, the disease is a neurological disorder.

In some embodiments, the neurological disorder is acute spinal cord injury (SCI), Alzheimer's Disease (AD), amyotrophic lateral sclerosis (ALS), ataxia, Bell's palsy, a brain tumor, cerebral aneurysm, epilepsy, Guillain-Barré syndrome (GBS), hydrocephalus, a lumbar disk disease, meningitis, multiple sclerosis (MS), muscular dystrophy, a neurocutaneous syndrome, Parkinson's disease (PD), stroke, a cluster headache, a tension headache, a migraine headache, encephalitis, septicemia or myasthenia gravis (MG), or a combination thereof. In some embodiments, the neurological disorder is AD or MS. In some embodiments, the neurological disorder is AD. In some embodiments, the neurological disorder is MS.

### Liver Diseases

In some embodiments, the disease is a liver disease.

In some embodiments, the liver disease is alagille syndrome (ALGS), autoimmune hepatitis (AIH), biliary atresia, cirrhosis, hemochromatosis, hepatitis, nonalcoholic fatty liver disease (NAFLD), primary biliary cholangitis (PBC), primary sclerosing cholangitis (PSC) or Wilson disease (WD), or a combination thereof. In some embodiments, the NAFLD is non-alcoholic steatohepatitis (NASH).

### Poly ADP Ribose Polymerase Inhibitors

As used herein, “a poly ADP ribose polymerase inhibitor” or “PARPi” refers to a substance that, when provided externally, results in the inhibition of poly ADP-ribose polymerase. PARPi includes any such substances currently known or future discovered, or a pharmaceutically acceptable salt, tautomer, N-oxide, solvate, hydrate or stereoisomer thereof.

In some embodiments, the PARPi is a poly [ADP-ribose] polymerase 1 (PARP1, also known as NAD⁺ ADP-ribosyltransferase 1 or poly[ADP-ribose] synthase 1) inhibitor. In some embodiments, the PARPi is a poly [ADP-ribose] polymerase 2 (PARP2) inhibitor. In some embodiments, the PARPi is a poly [ADP-ribose] polymerase 3 (PARP3) inhibitor. In some embodiments, the PARPi is a PARP1 inhibitor or a PARP2 inhibitor, or a combination thereof. In some embodiments, the PARPi is a PARP1 inhibitor, a PARP2 inhibitor or a PARP3 inhibitor, or a combination thereof. In some embodiments, the PARPi is a PARP4 inhibitor. In some embodiments, the PARPi is a PARP7 inhibitor. In some embodiments, the PARPi is a PARP14 inhibitor. In some embodiments, the PARPi is a PARP1 inhibitor, a PARP2 inhibitor, a PARP3 inhibitor, a PARP7 inhibitor or a PARP14 inhibitor, or a combination thereof. In some embodiments, the PARPi is a PARP1 inhibitor, a PARP2 inhibitor, a PARP3 inhibitor, a PARP4 inhibitor, a PARP7 inhibitor or a PARP14 inhibitor, or a combination thereof. In some embodiments, the PARPi is a PARP1 inhibitor, a PARP2 inhibitor, a PARP3 inhibitor, a PARP4 inhibitor, a PARP5 inhibitor, a PARP6 inhibitor, a PARP7 inhibitor, a PARP8 inhibitor, a PARP9 inhibitor, a PARP10 inhibitor, a PARP11 inhibitor, a PARP12 inhibitor, a PARP13 inhibitor, a PARP14 inhibitor, a PARP15 inhibitor, a PARP16 inhibitor or a PARP17 inhibitor, or a combination thereof.

In some embodiments, the PARPi is AG-14361 (CAS #328543-09-5), AZD2461 (CAS #1174043-16-3), CEP-8983 (CAS #374071-46-2), CEP-9722 (CAS #916574-83-9), E7016 (GPI21016, CAS #902128-92-1), iniparib (BSI 201, CAS #160003-66-7), INO-1001 (B2186, CAS #3544-24-9), niraparib (MK-4827, CAS #1038915-60-4), NU1025 (CAS #90417-38-2), olaparib (AZD-2281, Ku-0059436, CAS #763113-22-0), pamiparib (BGB-290, CAS #1446261-44-4), PJ34 (CAS #344458-15-7), PJ34HC1, RBN-2397 (CAS #2381037-82-5), rucaparib (AG-014447, CAS #283173-50-2; rucaparib phosphate (AG-014699, PF-01367338, CAS #459868-92-9)), talazoparib (BMN-673, CAS #1207456-01-6) or veliparib (ABT-888, CAS #912444-00-9), or a pharmaceutically acceptable salt, tautomer, N-oxide, solvate, hydrate or stereoisomer thereof. The CAS # refers to the Chemical Abstracts Registry Number.

In some embodiments, the disease is biliary duct cancer, bone cancer, breast cancer, colorectal cancer, endometrial cancer, fallopian tube cancer, hematologic cancer, lung cancer, melanoma, ovarian cancer, pancreatic cancer, peritoneal cancer, prostate cancer, sarcoma or skin cancer, or a combination thereof. See, e.g., Slade D., PARP and PARG inhibitors in cancer treatment, Genes Dev. 34(5-6):360-94 (2020) and Mateo J et al., A decade of clinical development of PARP inhibitors in perspective, Ann Oncol. 30(9):1437-47 (2019).

In some embodiments, the PARPi is NU1025, or a pharmaceutically acceptable salt thereof and the disease is cancer or cerebral ischemia.

In some embodiments, the PARPi is PJ34 or PJ34HC1, and the disease is alcoholic fatty liver disease, cancer, neurodegenerative diseases, retinal detachment or subarachnoid hemorrhage (SAH). In some embodiments, the cancer is breast cancer, colorectal cancer, glioblastoma, ovarian cancer or pancreas cancer. In some embodiments, the pancreas cancer is pancreatic ductal adenocarcinoma (PDAC).

In some embodiments, the PARPi is niraparib, olaparib, pamiparib, rucaparib, or talazoparib, or a pharmaceutically acceptable salt thereof.

Niraparib is indicated for the maintenance treatment of adult patients with recurrent epithelial ovarian, fallopian tube, or primary peritoneal cancer who are in a complete or partial response to platinum-based chemotherapy. ZEJULA™ (niraparib) is a capsule for oral use. In some embodiments, the dose is 300 mg taken once daily, with or without food. Additional information regarding niraparib can be found, for example, in the prescribing information product insert for ZEJULA™ (www.gsksource.com/pharma/content/dam/GlaxoSmithKline/US/en/Prescribing_Information/Zejula/pdf/ZEJULA-PI-PIL.PDF), which is incorporated herein in its entirety by reference.

Olaparib is indicated in ovarian cancer for the maintenance treatment of adult patients with recurrent epithelial ovarian, fallopian tube or primary peritoneal cancer, who are in a complete or partial response to platinum-based chemotherapy. Olaparib is indicated in ovarian cancer for the treatment of adult patients with deleterious or suspected deleterious germline BRCA-mutated (gBRCAm) advanced ovarian cancer who have been treated with three or more prior lines of chemotherapy. Olaparib is also indicated in breast cancer, in patients with deleterious or suspected deleterious gBRCAm, human epidermal growth factor receptor 2 (HER2)-negative metastatic breast cancer who have previously been treated with chemotherapy in the neoadjuvant, adjuvant or metastatic setting. Patients with hormone receptor (HR)-positive breast cancer should have been treated with a prior endocrine therapy or be considered inappropriate for endocrine treatment. LYNPARZA® (olaparib) is a tablet for oral use. In some embodiments, the tablet dose is 300 mg taken orally twice daily, with or without food. Additional information regarding olaparib can be found, for example, in the prescribing information product insert for LYNPARZA® (www.azpicentral.com/pi.html?product=lynparza_tb&country=us&popup=no), which is incorporated herein in its entirety by reference.

Rucaparib is indicated in ovarian cancer for the maintenance treatment of adult patients with recurrent epithelial ovarian, fallopian tube, or primary peritoneal cancer who are in a complete or partial response to platinum-based chemotherapy. Rucaparib is indicated in ovarian cancer for the treatment of adult patients with a deleterious BRCA mutation (germline and/or somatic)-associated epithelial ovarian, fallopian tube, or primary peritoneal cancer who have been treated with two or more chemotherapies. Rucaparib is also indicated in prostate cancer for the treatment of adult patients with a deleterious BRCA mutation (germline and/or somatic)-associated metastatic castration-resistant prostate cancer (mCRPC) who have been treated with androgen receptor-directed therapy and a taxane-based chemotherapy. Patients receiving rucaparib for mCRPC should also receive a gonadotropin-releasing hormone (GnRH) analog concurrently or should have had bilateral orchiectomy. RUBRACA® (rucaparib) is a tablet for oral use. In some embodiments, the dose is 600 mg orally, twice daily with or without food. Additional information regarding rucaparib can be found, for example, in the prescribing information product insert for RUBRACA® (clovisoncology.com/media/1094/rubraca-prescribing-info.pdf), which is incorporated herein in its entirety by reference.

Talazoparib is indicated for the treatment of adult patients with deleterious or suspected deleterious germline BRCA-mutated (gBRCAm) HER2-negative locally advanced or metastatic breast cancer. TALZENNA™ (talazoparib) is a capsule for oral use. In some embodiments, the dose of TALZENNA™ is 1 mg taken as a single oral daily dose, with or without food. Additional information regarding talazoparib can be found, for example, in the prescribing information product insert for TALZENNA™ (labeling.pfizer.com/ShowLabeling.aspx?id=11046), which is incorporated herein in its entirety by reference.

In some embodiments:
a) the PARPi is niraparib, or a stereoisomer, a tautomer, an N-oxide, a hydrate, a solvate or a pharmaceutically acceptable salt thereof, and the disease is biliary duct cancer, endometrial cancer, fallopian tube cancer, ovarian cancer, pancreatic cancer, peritoneal cancer, prostate cancer or skin cancer, or a combination thereof;
b) the PARPi is olaparib, or a stereoisomer, a tautomer, an N-oxide, a hydrate, a solvate or a pharmaceutically acceptable salt thereof, and the disease is biliary duct cancer, breast cancer, colorectal cancer, endometrial cancer, fallopian tube cancer, melanoma, ovarian cancer, pancreatic cancer, primary peritoneal cancer, prostate cancer or skin cancer, or a combination thereof;
c) the PARPi is pamiparib, or a stereoisomer, a tautomer, an N-oxide, a hydrate, a solvate or a pharmaceutically acceptable salt thereof, and the disease is esophageal cancer, glioma, head and neck cancer, non-small cell lung cancer (NSCLC), small cell gastric cancer, small cell lung cancer, soft tissue sarcoma or soft tissue sarcomas, or a combination thereof;
d) the PARPi is rucaparib, or a stereoisomer, a tautomer, an N-oxide, a hydrate, a solvate or a pharmaceutically acceptable salt thereof, and the disease is ovarian cancer; or
e) the PARPi is talazoparib, or a stereoisomer, a tautomer, an N-oxide, a hydrate, a solvate or a pharmaceutically acceptable salt thereof, and the disease is breast cancer, biliary duct cancer, bone cancer, colorectal cancer, endometrial cancer, lung cancer, pancreatic cancer, prostate cancer or skin cancer, or a combination thereof, or

a combination thereof.

In some embodiments, the PARPi is niraparib. In some embodiments, the disease is biliary duct cancer, endometrial cancer, fallopian tube cancer, ovarian cancer, pancreatic cancer, peritoneal cancer, prostate cancer or skin cancer. In some embodiments, the disease is recurrent epithelial ovarian, fallopian tube, or primary peritoneal cancer, and/or the subject is in a complete or partial response to platinum-based chemotherapy.

In some embodiments, the PARPi is olaparib. In some embodiments, the disease is biliary duct cancer, breast cancer, colorectal cancer, endometrial cancer, fallopian tube cancer, melanoma, ovarian cancer, pancreatic cancer, primary peritoneal cancer, prostate cancer or skin cancer. In some embodiments, the disease is ovarian cancer. In some embodiments, the subject is an adult patient with recurrent epithelial ovarian, fallopian tube or primary peritoneal cancer, and/or the subject is in a complete or partial response to platinum-based chemotherapy. In some embodiments, the subject is an adult patient with deleterious or suspected deleterious germline BRCA-mutated (gBRCAm) advanced ovarian cancer, and/or the subject has been treated with three or more prior lines of chemotherapy. In some embodiments, the disease is breast cancer, the subject is a patient with deleterious or suspected deleterious gBRCAm, human epidermal growth factor receptor 2 (HER2)-negative metastatic breast cancer, and/or the subject has previously been treated with chemotherapy in the neoadjuvant, adjuvant or metastatic setting. In some embodiments, the subject has hormone receptor (HR)-positive breast cancer, and/or the subject has been treated with a prior endocrine therapy or is considered inappropriate for endocrine treatment.

In some embodiments, the PARPi is pamiparib, and the disease is esophageal cancer, glioma, head and neck cancer, non-small cell lung cancer (NSCLC), small cell gastric cancer, small cell lung cancer, soft tissue sarcoma or soft tissue sarcomas.

In some embodiments, the PARPi is rucaparib. In some embodiments, the disease is ovarian cancer, and/or the subject is an adult patient with recurrent epithelial ovarian, fallopian tube, or primary peritoneal cancer who is in a complete or partial response to platinum-based chemotherapy. In some embodiments, the disease is ovarian cancer, and/or the subject is an adult patient with a deleterious BRCA mutation (germline and/or somatic)-associated epithelial ovarian, fallopian tube, or primary peritoneal cancer who have been treated with two or more chemotherapies. In some embodiments, the disease is prostate cancer, and/or the subject is an adult patient with a deleterious BRCA mutation (germline and/or somatic)-associated metastatic castration-resistant prostate cancer (mCRPC) who has been treated with androgen receptor-directed therapy and a taxane-based chemotherapy.

In some embodiments, the PARPi is talazoparib. In some embodiments, the disease is breast cancer, biliary duct cancer, bone cancer, colorectal cancer, endometrial cancer, lung cancer, pancreatic cancer, prostate cancer or skin cancer. In some embodiments, the subject is an adult patient with deleterious or suspected deleterious germline BRCA-mutated (gBRCAm) HER2-negative locally advanced or metastatic breast cancer.

In some embodiments, the bone cancer is Ewing sarcoma.

In some embodiments, the breast cancer is advanced breast cancer, BRCA1/2 mutated and human epidermal growth factor receptor type 2 (HER2)-negative metastatic breast cancer, or triple-negative breast cancer (TNBC).

In some embodiments, the lung cancer is small cell lung carcinoma.

In some embodiments, the ovarian cancer is advanced ovarian cancer, BRCA mutated ovarian cancer, high-grade epithelial ovarian cancer (HGOC), high-grade serous ovarian cancer, high-grade serous and undifferentiated ovarian cancer, platinum-sensitive, newly diagnosed advanced ovarian cancer, platinum-sensitive, relapsed ovarian cancer, platinum-sensitive, recurrent ovarian cancer, sporadic platinum-resistant high-grade serous ovarian cancer, relapsed high-grade ovarian carcinoma, relapsed, high-grade serous epithelial ovarian cancer or undifferentiated ovarian cancer.

In some embodiments, the pancreatic cancer is pancreatic adenocarcinoma or BRCA mutated metastatic pancreatic cancer.

In some embodiments, the prostate cancer is sporadic prostate cancer or metastatic, castration-resistant prostate cancer.

In some embodiments, the skin cancer is non-melanoma skin cancer.

In some embodiments, the anti-CD38 antibody (e.g., daratumumab or HexaBody-CD38 (GEN3014)) is administered in combination with the PARPi, i.e., the anti-CD38 antibody and PARPi are administered together in a mixture, concurrently as single agents or sequentially as single agents in any order. In some embodiments, the anti-CD38 antibody and the PARPi are administered in the same pharmaceutical composition.

In some embodiments, the anti-CD38 antibody and the PARPi are administered in different pharmaceutical compositions. In some embodiments, the anti-CD38 antibody and the PARPi are administered sequentially. In some embodiments, the PARPi is administered after the administration of the anti-CD38 antibody. In some embodiments, the PARPi is administered prior to the administration of the anti-CD38 antibody. In some embodiments, the anti-CD38 antibody and the PARPi are administered concurrently.

### Adenosine Receptor Antagonists

The term “adenosine receptor antagonist” refers to a substance that, when provided externally, acts against and blocks an action of an adenosine receptor. Adenosine receptor antagonist includes any such substances currently known or future discovered, or a pharmaceutically acceptable salt, tautomer, N-oxide, solvate, hydrate or stereoisomer thereof. See, e.g., Jacobson & Gao, Nat. Rev. Drug Discov. 5(3): 247-64 (2006) and Chen et al., Nat. Rev. Drug Discov. 12(4): 265-86 (2013).

In some embodiments, the adenosine receptor antagonist is an A₁AR antagonist, an A_{2A}AR antagonist, an A_{2B}AR antagonist or an A₃AR antagonist, or a combination thereof.

In some embodiments, the adenosine receptor antagonist is BG 9719, DPCPX (CAS #102146-07-6), FK453 (CAS #121524-18-3), FR194921 (CAS #202646-80-8), N-0861 (CAS #121241-87-0), rolofylline (KW 3902, CAS #136199-02-5), tonapofylline (BG 9928, CAS #340021-17-2) or WRC-0571 (CAS #501667-77-2), caffeine (CAS #58-08-2), 8-(-3-chlorostyryl)-caffeine (CSC, CAS #147700-11-6), istradefylline (KW-6002, CAS #155270-99-8), Preladenant (SCH 420814, CAS #377727-87-2), Schering compound, SCH 58261 (CAS #160098-96-4), SCH 442416 (CAS #316173-57-6), SYN115 (CAS #870070-55-6), VER-6947, VER-7835, ZM241,385 (CAS #139180-30-6), Eisai compound, MRE 2029-F20 (CAS #574753-99-4), MRS1754 (CAS #264622-58-4), OSIP-339391 (CAS #748136-54-1), FA385, MRE 3008-F20 (CAS #252979-43-4), MRS1292, MRS1334 (CAS #192053-05-7), MRS1523 (CAS #212329-37-8), MRS3777 (CAS #1186195-57-2), Novartis compound, OT-7999, PSB-11 (CAS #453591-58-7) or VUF5574 (CAS #280570-45-8), or a combination thereof.

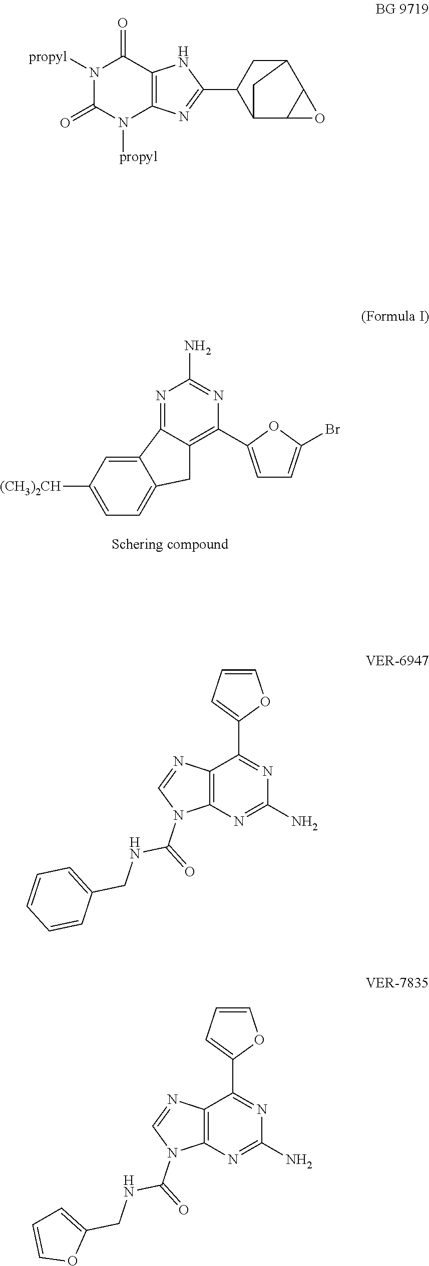

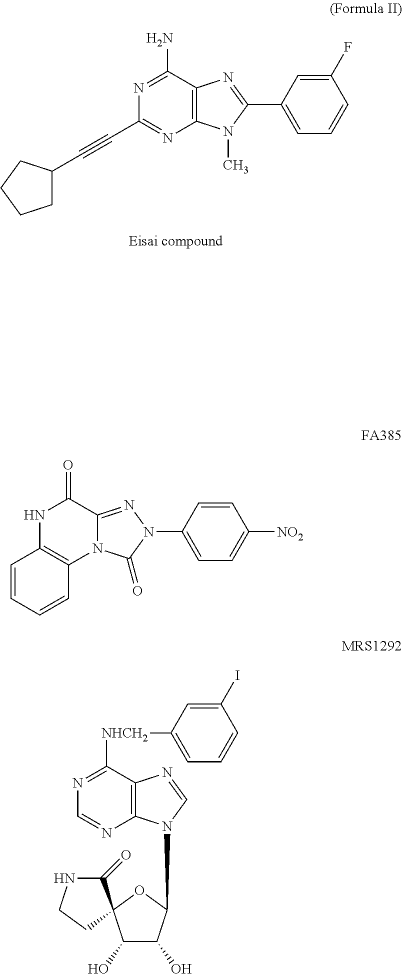

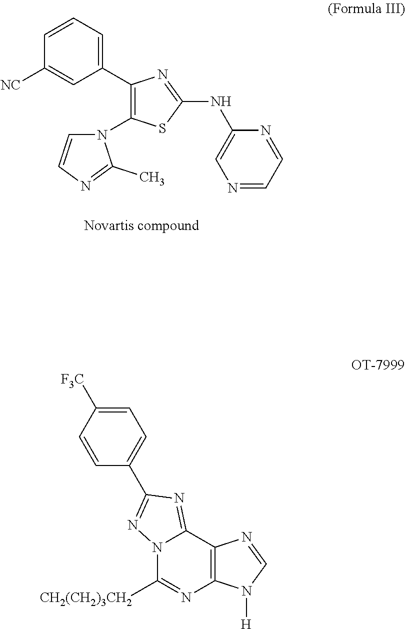

In some embodiments, the disease is anxiety disorder, cerebral ischemia, dementia, heart failure (e.g., acute heart failure), hepatic impairment, herniated lumbar disc, Parkinson's Disease (PD), renal insufficiency, restless legs syndrome, or a combination thereof.

In some embodiments, the adenosine receptor antagonist is an A₁AR antagonist. In some embodiments, the A₁AR antagonist displays at least 5-fold selectivity for human A₁AR versus human A_{2A}AR, for example, at least 10-, 15-, 20-, 50-, 100-, 150-, 200-, 250-, 300-, 350-, 400-, 450-, 500-, 550-, 600-, 650-, 700-, 750-, 800-, 850-, 900-, 950-, or 1000-fold selectivity for human A₁AR versus human A_{2A}AR. In some embodiments, the A₁AR antagonist displays at least 1.5-fold selectivity for human A₁AR versus human A_{2B}AR, for example, at least 2-, 3-, 4-, 5-, 6-, 7-, 8-, 9-, 10-, 11-, 12-, 13-, 14-, 15-, 16-, 17-, 18-, 19-, 20-, 25- or 30-fold selectivity for human A₁AR versus human A_{2B}AR. In some embodiments, the A₁AR antagonist displays at least 5-fold selectivity for human A₁AR versus human A₃AR, for example, at least 10-, 15-, 20-, 50-, 100-, 150-, 200-, 250-, 300-, 350-, 400-, 450-, 500-, 550-, 600-, 650-, 700-, 750-, 800-, 850-, 900-, 950-, or 1000-fold selectivity for human A₁AR versus human A₃AR.

In some embodiments, the A₁AR antagonist is BG 9719, DPCPX (CAS #102146-07-6), FK453 (CAS #121524-18-3), FR194921 (CAS #202646-80-8), N-0861 (CAS #121241-87-0), rolofylline (KW 3902, CAS #136199-02-5), tonapofylline (BG 9928, CAS #340021-17-2) or WRC-0571 (CAS #501667-77-2), or a combination thereof.

In some embodiments, the disease is heart failure (e.g., acute heart failure), renal insufficiency, hepatic impairment, dementia, anxiety disorder, or a combination thereof.

In some embodiments:
a) the adenosine receptor antagonist is BG 9719, or a stereoisomer, a tautomer, an N-oxide, a hydrate, a solvate or a pharmaceutically acceptable salt thereof, and the disease is renal insufficiency or congestive heart failure, or a combination thereof;
b) the adenosine receptor antagonist is FR194921, or a stereoisomer, a tautomer, an N-oxide, a hydrate, a solvate or a pharmaceutically acceptable salt thereof, and the disease is dementia or anxiety disorder, or a combination thereof;
c) the adenosine receptor antagonist is rolofylline (KW-3902), or a stereoisomer, a tautomer, an N-oxide, a hydrate, a solvate or a pharmaceutically acceptable salt thereof, and the disease is heart failure or renal insufficiency, or a combination thereof; or
d) the adenosine receptor antagonist is tonapofylline (BG 9928), or a stereoisomer, a tautomer, an N-oxide, a hydrate, a solvate or a pharmaceutically acceptable salt thereof, and the disease is heart failure, renal insufficiency or hepatic impairment, or a combination thereof, or

a combination thereof.

In some embodiments, heart failure is congestive heart failure or acute heart failure. In some embodiments, heart failure is acute heart failure.

In some embodiments, the adenosine receptor antagonist is BG 9719 and, for example, the disease is renal insufficiency or congestive heart failure.

In some embodiments, the adenosine receptor antagonist is FR194921 and, for example, the disease is dementia or anxiety disorder.

In some embodiments, the adenosine receptor antagonist is rolofylline (KW-3902), and, for example, the disease is heart failure or renal insufficiency. In some embodiments, the heart failure is congestive heart failure or acute heart failure.

In some embodiments, the adenosine receptor antagonist is tonapofylline (BG 9928), and, for example, the disease is heart failure, renal insufficiency or hepatic impairment. In some embodiments, the heart failure is acute heart failure.

In some embodiments, the adenosine receptor antagonist is non-selective for A₁AR and A_{2A}AR.

In some embodiments, the adenosine receptor antagonist is an A_{2A}AR antagonist. In some embodiments, the A_{2A}AR antagonist displays at least 5-fold selectivity for human A_{2A}AR versus human A₁AR, for example, at least 10-, 15-, 20-, 50-, 100-, 150-, 200-, 250-, 300-, 350-, 400-, 450-, 500-, 1,000-, 2,000-, 4,000-, 5,000-, 10,000-, 20,000- or 30,000-fold selectivity for human A_{2A}AR versus human A₁AR. In some embodiments, the A_{2A}AR antagonist displays at least 5-fold selectivity for human A_{2A}AR versus human A_{2B}AR, for example, at least 10-, 15-, 20-, 25-, 30-, 35-, 40-, 45-, 50-, 55-, 60-, 65-, 70-, 75-, 80-, 85-, 90-, 95-, 100-, 200-, 250-, 300-, 350-, 400-, 450-, 500-, 1,000-, 2,000-, 4,000-, 5,000-, 10,000-, 20,000-, 50,000- or 100,000-fold selectivity for human A_{2A}AR versus human A_{2B}AR. In some embodiments, the A_{2A}AR antagonist displays at least 5-fold selectivity for human A_{2A}AR versus human A₃AR, for example, at least 10-, 15-, 20-, 30-, 40-, 50-, 60-, 70-, 80-, 90-, 100-, 125-, 150-, 175-, 200-, 250-, 300-, 350-, 400-, 450-, 500-, 1,000-, 2,000-, 4,000-, 5,000-, 10,000-, 20,000-, 50,000- or 100,000-fold selectivity for human A_{2A}AR versus human A₃AR.

In some embodiments, the adenosine receptor antagonist is caffeine (CAS #58-08-2), 8-(-3-chlorostyryl)-caffeine (CSC, CAS #147700-11-6), istradefylline (KW-6002, CAS #155270-99-8), Preladenant (SCH 420814, CAS #377727-87-2), Schering compound, SCH 58261 (CAS #160098-96-4), SCH 442416 (CAS #316173-57-6), SYN115 (CAS #870070-55-6), VER-6947, VER-7835 or ZM241,385 (CAS #139180-30-6), or a combination thereof.

In some embodiments, the adenosine receptor antagonist is istradefylline.

Istradefylline is indicated as adjunctive treatment to levodopa/carbidopa in adult patients with Parkinson's disease (PD) experiencing “off” episodes. NOURIANZ™ (istradefylline) is a tablet for oral use. In some embodiments, the dosage is 20 mg orally once daily. The dosage may be increased to a maximum of 40 mg once daily. Additional information regarding istradefylline can be found, for example, in the prescribing information product insert for NOURIANZ™ (https://www.nourianzhcp.com/assets/pdf/nourianz-full-prescribing-information.pdf), which is incorporated herein in its entirety by reference.

In some embodiments, the disease is selected from the group consisting of Parkinson's Disease (PD), restless legs syndrome, cerebral ischemia, herniated lumbar disc, and combinations thereof.

In some embodiments:
a) the adenosine receptor antagonist is caffeine or a stereoisomer, a tautomer, an N-oxide, a hydrate, a solvate or a pharmaceutically acceptable salt thereof, and the disease is Parkinson's Disease (PD);
e) the adenosine receptor antagonist is istradefylline (KW-6002) or a stereoisomer, a tautomer, an N-oxide, a hydrate, a solvate or a pharmaceutically acceptable salt thereof, and the disease is Parkinson's Disease (PD) or restless legs syndrome, or a combination thereof
f) the adenosine receptor antagonist is Preladenant (SCH 420814) or a stereoisomer, a tautomer, an N-oxide, a hydrate, a solvate or a pharmaceutically acceptable salt thereof, and the disease is Parkinson's Disease (PD);
g) the adenosine receptor antagonist is Schering compound or a stereoisomer, a tautomer, an N-oxide, a hydrate, a solvate or a pharmaceutically acceptable salt thereof, and the disease is herniated lumbar disc;
h) the adenosine receptor antagonist is SCH 58261 or a stereoisomer, a tautomer, an N-oxide, a hydrate, a solvate or a pharmaceutically acceptable salt thereof, and the disease is cerebral ischaemia;
i) the adenosine receptor antagonist is SCH 442416 or a stereoisomer, a tautomer, an N-oxide, a hydrate, a solvate or a pharmaceutically acceptable salt thereof, and the disease is Parkinson's Disease (PD); or
j) the adenosine receptor antagonist is SYN115 or a stereoisomer, a tautomer, an N-oxide, a hydrate, a solvate or a pharmaceutically acceptable salt thereof, and the disease is Parkinson's Disease (PD), or

a combination thereof.

In some embodiments, the adenosine receptor antagonist is caffeine. In some embodiments, the disease is Parkinson's Disease (PD).

In some embodiments, the adenosine receptor antagonist is istradefylline. In some embodiments, the disease is Parkinson's Disease (PD) or restless legs syndrome. In some embodiments, the subject is an adult patient with Parkinson's disease (PD) treated with levodopa/carbidopa and experiences one or more “off” episodes.

In some embodiments, the adenosine receptor antagonist is Preladenant (SCH 420814). In some embodiments, the disease is Parkinson's Disease (PD).

In some embodiments, the adenosine receptor antagonist is Schering compound. In some embodiments, the disease is herniated lumbar disc.

In some embodiments, the adenosine receptor antagonist is SCH 58261. In some embodiments, the disease is cerebral ischemia (i.e., ischaemia).

In some embodiments, the adenosine receptor antagonist is SCH 442416. In some embodiments, the disease is Parkinson's Disease (PD).

In some embodiments, the adenosine receptor antagonist is SYN115. In some embodiments, the disease is Parkinson's Disease (PD).

In some embodiments, the adenosine receptor antagonist is an A_{2B}AR antagonist. In some embodiments, the A_{2B}AR antagonist displays at least 5-fold selectivity for human A_{2B}AR versus human A₁AR, for example, at least 10-, 15-, 20-, 50-, 100-, 150-, 200-, 250-, 300-, 350-, 400-, 450- or 500-fold selectivity for human A_{2B}AR versus human A₁AR. In some embodiments, the A_{2B}AR antagonist displays at least 5-fold selectivity for human A_{2B}AR versus human A_{2A}AR, for example, at least 10-, 15-, 20-, 25-, 30-, 35-, 40-, 45-, 50-, 55-, 60-, 65-, 70-, 75-, 80-, 85-, 90-, 95-, 100-, 200-, 250-, 300-, 350-, 400-, 450-, 500-, 750- or 1,000-fold selectivity for human A_{2B}AR versus human A_{2A}AR. In some embodiments, the A_{2B}AR antagonist displays at least 5-fold selectivity for human A_{2B}AR versus human A₃AR, for example, at least 10-, 15-, 20-, 30-, 40-, 50-, 60-, 70-, 80-, 90-, 100-, 125-, 150-, 175-, 200-, 250-, 300-, 350-, 400-, 450-, 500-, 750- or 1,000-fold selectivity for human A_{2B}AR versus human A₃AR.

In some embodiments, the adenosine receptor antagonist is Eisai compound, MRE 2029-F20 (CAS #574753-99-4), MRS1754 (CAS #264622-58-4) or OSIP-339391 (CAS #748136-54-1), or a combination thereof.

In some embodiments, the adenosine receptor antagonist is an A₃AR antagonist. In some embodiments, the A₃AR antagonist displays at least 5-fold selectivity for human A₃AR versus human A₁AR, for example, at least 10-, 15-, 20-, 50-, 100-, 150-, 200-, 250-, 300-, 350-, 400-, 450-, 500-, 1,000-, 2,000-, 4,000-, 5,000-, 10,000-, 20,000- or 30,000-fold selectivity for human A₃AR versus human A₁AR. In some embodiments, the A₃AR antagonist displays at least 5-fold selectivity for human A₃AR versus human A_{2A}AR, for example, at least 10-, 15-, 20-, 50-, 100-, 150-, 200-, 250-, 300-, 350-, 400-, 450-, 500-, 1,000-, 2,000-, 4,000-, 5,000-, 10,000-, 20,000- or 30,000-fold selectivity for human A₃AR versus human A_{2A}AR. In some embodiments, the A₃AR antagonist displays at least 5-fold selectivity for human A₃AR versus human A_{2B}AR, for example, at least 10-, 15-, 20-, 50-, 100-, 150-, 200-, 250-, 300-, 350-, 400-, 450-, 500-, 1,000-, 2,000-, 4,000-, 5,000-, 10,000-, 20,000- or 30,000-fold selectivity for human A₃AR versus human A_{2B}AR.

In some embodiments, the adenosine receptor antagonist is FA385, MRE 3008-F20 (CAS #252979-43-4), MRS1292, MRS1334 (CAS #192053-05-7), MRS1523 (CAS #212329-37-8), MRS3777 (CAS #1186195-57-2), Novartis compound, OT-7999, PSB-11 (CAS #453591-58-7) or VUF5574 (CAS #280570-45-8), or a combination thereof.

In some embodiments, the anti-CD38 antibody (e.g., daratumumab or HexaBody-CD38 (GEN3014)) is administered in combination with the adenosine receptor antagonist. In some embodiments, the anti-CD38 antibody and the adenosine receptor antagonist are administered in the same pharmaceutical composition. In some embodiments, the anti-CD38 antibody and the adenosine receptor antagonist are administered concurrently as single agents.

In some embodiments, the anti-CD38 antibody and the adenosine receptor antagonist are administered in different pharmaceutical compositions. In some embodiments, the anti-CD38 antibody and the adenosine receptor antagonist are administered sequentially as single agents. In some embodiments, the adenosine receptor antagonist is administered prior to the administration of the anti-CD38 antibody. In some embodiments, the adenosine receptor antagonist is administered after the administration of the anti-CD38 antibody.

In some embodiments, the method comprises administering to the subject an anti-CD38 antibody, a PARPi and an adenosine receptor antagonist for a time sufficient to treat the disease.

Also included are uses of one or more of the compounds (e.g., a PARPi, an adenosine receptor antagonist, or both and an anti-CD38 antibody) or compositions recited herein for treatment, or for manufacture of a medicament for treatment, of a disease or disorder provided herein.

### Combination Therapies

In some embodiments of the invention, the subject has cancer (e.g., a solid tumor), and the PARPi, adenosine receptor antagonist, or both and the anti-CD38 antibody is administered in combination with a chemotherapeutic agent, a targeted anti-cancer therapy, a standard of care drug for treatment of cancer, or an immune checkpoint inhibitor.

In some embodiments, the PARPi and the chemotherapeutic agent, targeted anti-cancer therapy, standard of care drug for treatment of cancer, or immune checkpoint inhibitor are administered simultaneously. In some embodiments, the PARPi and the chemotherapeutic agent, targeted anti-cancer therapy, standard of care drug for treatment of cancer, or immune checkpoint inhibitor are administered sequentially or separately.

In some embodiments, the adenosine receptor antagonist and the chemotherapeutic agent, targeted anti-cancer therapy, standard of care drug for treatment of cancer, or immune checkpoint inhibitor are administered simultaneously. In some embodiments, the adenosine receptor antagonist and the chemotherapeutic agent, targeted anti-cancer therapy, standard of care drug for treatment of cancer, or immune checkpoint inhibitor are administered sequentially or separately.

In some embodiments, the anti-CD38 antibody and the chemotherapeutic agent, targeted anti-cancer therapy, standard of care drug for treatment of cancer, or immune checkpoint inhibitor are administered simultaneously. In some embodiments, the anti-CD38 antibody and the chemotherapeutic agent, targeted anti-cancer therapy, standard of care drug for treatment of cancer, or immune checkpoint inhibitor are administered sequentially or separately.

In some embodiments, the immune checkpoint inhibitor is an anti-PD-1 antibody, an anti-PD-L1 antibody, an anti-PD-L2 antibody, an anti-LAG3 antibody, an anti-TIM3 antibody, or an anti-CTLA-4 antibody.

In some embodiments, the immune checkpoint inhibitor is an anti-PD-1 antibody. In some embodiments, the anti-PD-1 antibody comprises a VH and VL amino acid sequences of:
a) SEQ ID NO: 23 and SEQ ID NO: 24, respectively;
b) SEQ ID NO: 25 and SEQ ID NO: 26, respectively;
c) SEQ ID NO: 33 and SEQ ID NO: 34, respectively; or
d) SEQ ID NO: 35 and SEQ ID NO:36, respectively.

In some embodiments, the immune checkpoint inhibitor is an anti-PD-L1 antibody. In some embodiments, the anti-PD-L1 antibody comprises a VH and VL amino acid sequences of:
a) SEQ ID NO: 27 and SEQ ID NO: 28, respectively;
b) SEQ ID NO: 29 and SEQ ID NO: 30, respectively; or
c) SEQ ID NO: 31 and SEQ ID NO: 32, respectively.

In some embodiments, the immune checkpoint inhibitor is an anti-PD-L2 antibody.

In some embodiments, the immune checkpoint inhibitor is an anti-LAG3 antibody. Non-limiting examples of anti-LAG-3 antibodies include those described in Int. Pat. Publ. No. WO2010/019570.

In some embodiments, the immune checkpoint inhibitor is an anti-TIM-3 antibody. In some embodiments, the anti-T1M-3 antibody comprises a VH and VL amino acid sequences of:
a) SEQ ID NO: 37 and SEQ ID NO: 38, respectively; or
b) SEQ ID NO: 39 and SEQ ID NO: 40, respectively.

In some embodiments, the immune checkpoint inhibitor is an anti-CTLA-4 antibody. A non-limiting example of anti-CTLA-4 antibodies is Ipilimumab.

The anti-PD-1, anti-PD-L1, anti-PD-L2, anti-LAG3, anti-TIM3 and anti-CTLA-4 antibodies may be generated de novo.

In some embodiments, the anti-CD38 antibody and the immune checkpoint inhibitor are administered simultaneously. In some embodiments, the anti-CD38 antibody and the immune checkpoint inhibitor are administered sequentially or separately.

In some embodiments, the method of the invention further comprises administering a form of radiation therapy, surgery or a combination thereof. Non-limiting examples of radiation therapies include external beam radiation, intensity modulated radiation therapy (IMRT), focused radiation, and any form of radiosurgery including Gamma Knife, Cyberknife, Linac, and interstitial radiation (e.g., implanted radioactive seeds, GliaSite balloon).

Focused radiation methods that may be used include stereotactic radiosurgery, fractionated stereotactic radiosurgery, and intensity-modulated radiation therapy (IMRT). It is apparent that stereotactic radiosurgery involves the precise delivery of radiation to a tumorous tissue, for example, a brain tumor, while avoiding the surrounding nontumorous, normal tissue. The dosage of radiation applied using stereotactic radiosurgery may vary, typically from 1 Gy to about 30 Gy, and may encompass intermediate ranges including, for example, from 1 to 5, 10, 15, 20, 25, up to 30 Gy in dose. Because of noninvasive fixation devices, stereotactic radiation need not be delivered in a single treatment. The treatment plan may be reliably duplicated day-to-day, thereby allowing multiple fractionated doses of radiation to be delivered. When used to treat a tumor over time, the radiosurgery is referred to as “fractionated stereotactic radiosurgery” or FSR. In contrast, stereotactic radiosurgery refers to a one-session treatment. Fractionated stereotactic radiosurgery may result in a high therapeutic ratio, i.e., a high rate of killing of tumor cells and a low effect on normal tissue. The tumor and the normal tissue respond differently to high single doses of radiation vs. multiple smaller doses of radiation. Single large doses of radiation may kill more normal tissue than several smaller doses of radiation may. Accordingly, multiple smaller doses of radiation can kill more tumor cells while sparing normal tissue. The dosage of radiation applied using fractionated stereotactic radiation may vary from range from 1 Gy to about 50 Gy, and may encompass intermediate ranges including, for example, from 1 to 5, 10, 15, 20, 25, 30, 40, up to 50 Gy in hypofractionated doses. Intensity-modulated radiation therapy (IMRT) may also be used. IMRT is an advanced mode of high-precision three-dimensional conformal radiation therapy (3DCRT), which uses computer-controlled linear accelerators to deliver precise radiation doses to a malignant tumor or specific areas within the tumor. In 3DCRT, the profile of each radiation beam is shaped to fit the profile of the target from a beam's eye view (BEV) using a multileaf collimator (MLC), thereby producing a number of beams. IMRT allows the radiation dose to conform more precisely to the three-dimensional (3-D) shape of the tumor by modulating the intensity of the radiation beam in multiple small volumes. Accordingly, IMRT allows higher radiation doses to be focused to regions within the tumor while minimizing the dose to surrounding normal critical structures. IMRT improves the ability to conform the treatment volume to concave tumor shapes, for example, when the tumor is wrapped around a vulnerable structure, such as the spinal cord or a major organ or blood vessel.

In some embodiments of the invention, the subject has cancer (e.g., AL), and the subject undergoes a hematopoietic stem cell transplantation (HSCT). “Hematopoietic stem cell transplantation” is the transplantation of blood stem cells derived from the bone marrow (in this case known as bone marrow transplantation), blood (such as peripheral blood and umbilical cord blood), or amniotic fluid. Undergoing hematopoietic stem cell transplantation” means that the patient did already receive, is receiving or will receive HSCT.

In some embodiments, the HSCT is allogeneic. In some embodiments, the HSCT is autologous or syngeneic (i.e., the donor is a twin). Autologous HSCT comprises the extraction of HSC from the subject and freezing of the harvested HSC. After myeloablation, the subject's stored HSC are transplanted into the subject. Allogeneic HSCT involves HSC obtained from an allogeneic HSC donor who has an HLA type that matches the subject.

In some embodiments, the subject has completed chemotherapy and/or radiation therapy prior to HSCT.

Patients may be treated with chemotherapy and/or radiation therapy prior to HSCT (so-called pre-transplant preparation) to eradicate some or all of the patient's hematopoietic cells prior to transplant. The patient may also be treated with immunosuppressants in case of allogeneic HSCT. An exemplary pre-transplant preparation therapy is high-dose melphalan (see, e.g., Skinner et al., Ann. Intern. Med. 140:85-93 (2004), Gertz et al., Bone Marrow Transplant 34:1025-31 (2004), Perfetti et al., Haematologica 91:1635-43 (2006)). The radiation therapy that may be employed in pre-transplant treatment may be carried out according to protocols commonly known in this field. Radiation therapy may be provided simultaneously, sequentially or separately with the anti-CD38 antibody.

In some embodiments (e.g., treating AL), the method further comprises administering to the subject a proteasome inhibitor, a corticosteroid and a cyclophosphamide for a time sufficient to treat the disease or condition (e.g., AL). In some embodiments, the proteasome inhibitor is Velcade® (bortezomib), or vinca alkaloids, for example vincristine or an anthracycline, such as doxorubicin. In some embodiments, the proteasome inhibitor is Velcade® (bortezomib). In some embodiments, the corticosteroid is dexamethasone. In some embodiments, the corticosteroid is prednisone.

Cyclophosphamide may be administered IV (intermittent therapy) 40-50 mg/kg (400-1800 mg/m²) divided over 2-5 days; may be repeated at intervals of 2-4 weeks; IV (continuous daily therapy): 60-120 mg/m²/day (1-2.5 mg/kg/day); PO (intermittent therapy): 400-1000 mg/m² divided over 4-5 days or PO (continuous daily therapy): 50-100 mg/m²/day or 1-5 mg/kg/day.

Bortezomib may be administered at 1.3 mg/m² SQ twice weekly or once weekly.

Dexamethasone may be administered 40 mg/week, or 20 mg pre- and post-dose with the anti-CD38 antibody.

In some embodiments, the method comprises administering to the subject an anti-CD38 antibody (e.g., daratumumab) and CyBorD (cyclophosphamide, bortezomib and dexamethasone), for a time sufficient to treat the disease or condition (e.g., AL). In some embodiments, cyclophosphamide is administered at 300 mg/m² (oral or IV), bortezomib is administered at 1.3 mg/m² (SC injection), and dexamethasone is administered at 20 mg (oral or IV) as premedication and 20 mg on the day after daratumumab dosing.

While example embodiments have been particularly shown and described, it will be understood by those skilled in the art that various changes in form and details may be made therein without departing from the scope of the embodiments encompassed by the appended claims.

### EMBODIMENTS

1. A method of treating a disease in a subject in need thereof, comprising administering to the subject an anti-CD38 antibody and a poly ADP ribose polymerase inhibitor (PARPi) for a time sufficient to treat the disease.
2. The method of Embodiment 1, wherein the anti-CD38 antibody comprises:
   a) a heavy chain complementarity determining region 1 (HCDR1), HCDR2 and HCDR3 amino acid sequences of SEQ ID NOs: 6, 7 and 8, respectively; and
   b) a light chain complementarity determining region 1 (LCDR1), LCDR2 and LCDR3 amino acid sequences of SEQ ID NOs: 9, 10 and 11, respectively.
3. The method of Embodiment 1, wherein the anti-CD38 antibody comprises:
   a) a heavy chain variable region (VH) sequence of SEQ ID NO: 4; and
   b) a light chain variable region (VL) sequence of SEQ ID NO: 5.
4. The method of Embodiment 1, wherein the anti-CD38 antibody comprises a heavy chain sequence of SEQ ID NO: 12 and a light chain sequence of SEQ ID NO: 13.
5. The method of Embodiment 1, wherein the anti-CD38 antibody comprises:
   a) the heavy chain complementarity determining region 1 (HCDR1), HCDR2 and HCDR3 amino acid sequences of the heavy chain variable region (VH) of SEQ ID NO: 14 and the light chain complementarity determining region 1 (LCDR1), LCDR2, and LCDR3 amino acid sequences of the variable region (VL) of SEQ ID NO: 15;
   b) the HCDR1, HCDR2 and HCDR3 amino acid sequences of the VH of SEQ ID NO: 16 and the LCDR1, LCDR2, and LCDR3 amino acid sequences of the VL of SEQ ID NO: 17;
   c) the HCDR1, HCDR2 and HCDR3 amino acid sequences of the VH of SEQ ID NO: 18 and the LCDR1, LCDR2, and LCDR3 amino acid sequences of the VL of SEQ ID NO: 19; or
   d) the HCDR1, HCDR2 and HCDR3 amino acid sequences of the VH of SEQ ID NO: 20 and the LCDR1, LCDR2, and LCDR3 amino acid sequences of the VL of SEQ ID NO: 21.
6. The method of Embodiment 5, wherein the anti-CD38 antibody comprises the VH and VL sequences of:
   a) SEQ ID NOs: 14 and 15, respectively;
   b) SEQ ID NOs: 16 and 17, respectively;
   c) SEQ ID NOs: 18 and 19, respectively; or
   d) SEQ ID NOs: 20 and 21, respectively.
7. The method of any one of Embodiments 1-6, wherein the anti-CD38 antibody is of the IgG1, IgG2, IgG3 or IgG4 subtype.
8. The method of Embodiment 7, wherein the anti-CD38 antibody is of the IgG1 subtype.
9. The method of Embodiment 8, wherein the anti-CD38 antibody is of the IgG1/κ subtype.
10. The method of Embodiment 1, wherein the anti-CD38 antibody is daratumumab.
11. The method of any one of Embodiments 1-10, wherein the anti-CD38 antibody is administered intravenously.
12. The method of any one of Embodiments 1-10, wherein the anti-CD38 antibody is administered subcutaneously.
13. The method of Embodiment 12, wherein the anti-CD38 antibody is administered in a pharmaceutical composition comprising the anti-CD38 antibody and a hyaluronidase.
14. The method of Embodiment 13, wherein the hyaluronidase is rHuPH20 and has the amino acid sequence of SEQ ID NO: 22.
15. The method of any one of Embodiments 1-14, wherein the disease is cancer.
16. The method of Embodiment 15, wherein the cancer is a hematologic cancer.
17. The method of Embodiment 16, wherein the hematologic cancer is leukemia.
18. The method of Embodiment 17, wherein the leukemia is acute lymphoblastic leukemia (ALL), acute myeloid leukemia (AML), chronic lymphocytic leukemia (CLL), chronic myeloid leukemia (CML), hairy cell leukemia (HCL) or myelodysplastic syndromes (MDS).
19. The method of Embodiment 16, wherein the hematologic cancer is lymphoma.
20. The method of Embodiment 19, wherein the lymphoma is Hodgkin lymphoma.
21. The method of Embodiment 20, wherein the Hodgkin lymphoma is nodular sclerosis Hodgkin lymphoma (NSCHL), mixed cellularity Hodgkin lymphoma (MCcHL), lymphocyte-rich Hodgkin's disease (LRCHL) or lymphocyte-depleted Hodgkin's disease (LDHL).
22. The method of Embodiment 19, wherein the lymphoma is non-Hodgkin lymphoma (NHL).
23. The method of Embodiment 22, wherein the non-Hodgkin lymphoma is a B cell lymphoma.
24. The method of Embodiment 23, wherein the B cell lymphoma is diffuse large B-cell lymphoma (DLBCL), primary mediastinal B cell lymphoma (PMBCL), follicular lymphoma (FL), small lymphocytic lymphoma (SLL), marginal zone lymphoma (MZL), mantle cell lymphoma (MCL), Waldenström's macroglobulinemia (WMG) or Burkitt lymphoma (BL).
25. The method of Embodiment 22, wherein the non-Hodgkin lymphoma is a T cell lymphoma.
26. The method of Embodiment 25, wherein the T cell lymphoma is peripheral T-cell lymphoma (PTCL), anaplastic large cell lymphoma (ALCL), angioimmunoblastic T-cell lymphoma (AITL) or cutaneous T cell lymphoma.
27. The method of Embodiment 16, wherein the hematologic cancer is multiple myeloma.
28. The method of Embodiment 27, wherein the multiple myeloma is light chain multiple myeloma (LCMM), non-secretory multiple myeloma (NSMM), solitary plasmacytoma (SP), extramedullary plasmacytoma (EMP), monoclonal gammopathy of undetermined significance (MGUS), smoldering Multiple Myeloma (SMM), Immunoglobulin D multiple myeloma (IgD MM) or Immunoglobulin E (IgE) multiple myeloma.
29. The method of Embodiment 16, wherein the hematologic cancer is a CD38-positive hematological malignancy.
30. The method of Embodiment 29, wherein the CD38-positive hematological malignancy is multiple myeloma (MM), acute lymphoblastic leukemia (ALL), non-Hodgkin's lymphoma (NHL), diffuse large B-cell lymphoma (DLBCL), Burkitt's lymphoma (BL), follicular lymphoma (FL), mantle-cell lymphoma (MCL), acute myeloid leukemia (AML) or chronic lymphocytic leukemia (CLL).
31. The method of Embodiment 29, wherein the CD38-positive hematological malignancy is a plasma cell disease.
32. The method of Embodiment 31, wherein the plasma cell disease is light chain amyloidosis (AL), multiple myeloma (MM) or Waldenström's macroglobulinemia.
33. The method of Embodiment 32, wherein the plasma cell disease is MM.
34. The method of Embodiment 32, wherein the plasma cell disease is AL.
35. The method of Embodiment 15, wherein the cancer is a solid tumor.
36. The method of Embodiment 35, wherein the solid tumor is a tumor of the breast, lung, prostate, colon, bladder, ovary, kidney, stomach, colon, rectum, testes, head and/or neck, pancreas, brain or skin.
37. The method of Embodiment 35, wherein the solid tumor is bladder cancer, brain cancer, breast cancer, cervical cancer, colon cancer, colorectal cancer, fallopian tube cancer, gastric cancer, genitourinary cancer, head and neck cancer, liver cancer, lung cancer, melanoma, nasopharyngeal carcinoma (NPC), pancreatic cancer, prostate cancer, ovarian cancer, rectal cancer, renal cancer, skin cancer, stomach cancer, testicular cancer, thyroid cancer or urethral cancer.
38. The method of Embodiment 35, wherein the solid tumor is squamous non-small cell lung cancer (NSCLC), non-squamous NSCLC, lung adenocarcinoma, mesothelioma, kidney clear cell carcinoma, kidney papillary cell carcinoma, castration-resistant prostate cancer, squamous cell carcinoma of the head and neck, carcinomas of the esophagus, carcinomas of the gastrointestinal tract or endometriosis.
39. The method of any one of Embodiments 35-38, wherein the solid tumor is a metastatic lesion of the cancer.
40. The method of any one of Embodiments 1-14, wherein the disease is a neurological disorder.
41. The method of Embodiment 40, wherein the neurological disorder is acute spinal cord injury (SCI), Alzheimer's Disease (AD), amyotrophic lateral sclerosis (ALS), ataxia, Bell's palsy, a brain tumor, cerebral aneurysm, epilepsy, Guillain-Barré syndrome (GBS), hydrocephalus, a lumbar disk disease, meningitis, multiple sclerosis (MS), muscular dystrophy, a neurocutaneous syndrome, Parkinson's disease (PD), stroke, a cluster headache, a tension headache, a migraine headache, encephalitis, septicemia or myasthenia gravis (MG).
42. The method of Embodiment 41, wherein the neurological disorder is Alzheimer's Disease (AD) or multiple sclerosis (MS).
43. The method of any one of Embodiments 1-14, wherein the disease is a liver disease.
44. The method of Embodiment 43, wherein the liver disease is alagille syndrome (ALGS), autoimmune hepatitis (AIH), biliary atresia, cirrhosis, hemochromatosis, hepatitis, nonalcoholic fatty liver disease (NAFLD), primary biliary cholangitis (PBC), primary sclerosing cholangitis (PSC) or Wilson disease (WD).
45. The method of Embodiment 44, wherein the NAFLD is non-alcoholic steatohepatitis (NASH).
46. The method of any one of Embodiments 1-45, wherein the PARPi is a PARP1 inhibitor, a PARP2 inhibitor or a PARP3 inhibitor.
47. The method of any one of Embodiments 1-45, wherein the PARPi is AZD2461, CEP-8983, CEP-9722, E7016 (GPI21016), Iniparib (BSI 201), INO-1001, Niraparib (MK-4827), Olaparib (AZD-2281), Pamiparib (BGB-290), Rucaparib (AG-014699, PF-01367338), Talazoparib (BMN-673) or Veliparib (ABT-888).
48. The method of Embodiment 47, wherein the PARPi is Niraparib (MK-4827), Olaparib (AZD-2281), Rucaparib (AG-014699, PF-01367338), or Talazoparib (BMN-673).
49. The method of any one of Embodiments 1-48, wherein the disease is biliary duct cancer, bone cancer, breast cancer, colorectal cancer, endometrial cancer, fallopian tube cancer, hematologic cancer, lung cancer, melanoma, ovarian cancer, pancreatic cancer, peritoneal cancer, prostate cancer, sarcoma or skin cancer.
50. The method of Embodiment 1-47, wherein:
   a) the PARPi is Niraparib, and the disease is biliary duct cancer, endometrial cancer, fallopian tube cancer, ovarian cancer, pancreatic cancer, peritoneal cancer, prostate cancer or skin cancer;
   b) the PARPi is Olaparib, and the disease is biliary duct cancer, breast cancer, colorectal cancer, endometrial cancer, fallopian tube cancer, melanoma, ovarian cancer, pancreatic cancer, primary peritoneal cancer, prostate cancer or skin cancer;
   c) the PARPi is Pamiparib (BGB-290), and the disease is esophageal cancer, glioma, head and neck cancer, non-small cell lung cancer (NSCLC), small cell gastric cancer, small cell lung cancer or soft tissue sarcomas;
   d) the PARPi is Rucaparib, and the disease is ovarian cancer; or
   e) the PARPi is Talazoparib, and the disease is breast cancer, biliary duct cancer, bone cancer, colorectal cancer, endometrial cancer, lung cancer, pancreatic cancer, prostate cancer or skin cancer.
51. The method of Embodiment 49 or 50, wherein:
   a) the bone cancer is Ewing sarcoma;
   b) the breast cancer is advanced breast cancer, BRCA1/2 mutated and human epidermal growth factor receptor type 2 (HER2)-negative metastatic breast cancer, or triple-negative breast cancer (TNBC);
   c) the lung cancer is small cell lung carcinoma;
   d) the ovarian cancer is advanced ovarian cancer, BRCA mutated ovarian cancer, high-grade epithelial ovarian cancer (HGOC), high-grade serous ovarian cancer, high-grade serous and undifferentiated ovarian cancer, platinum-sensitive, newly diagnosed advanced ovarian cancer, platinum-sensitive, relapsed ovarian cancer, platinum-sensitive, recurrent ovarian cancer, sporadic platinum-resistant high-grade serous ovarian cancer, relapsed high-grade ovarian carcinoma, relapsed, high-grade serous epithelial ovarian cancer or undifferentiated ovarian cancer;
   e) the pancreatic cancer is pancreatic adenocarcinoma or BRCA mutated metastatic pancreatic cancer;
   f) the prostate cancer is sporadic prostate cancer or metastatic, castration-resistant prostate cancer; or
   g) the skin cancer is non-melanoma skin cancer.
52. The method of any one of Embodiments 1-51, wherein the anti-CD38 antibody and the PARPi are administered simultaneously.
53. The method of any one of Embodiments 1-51, wherein the anti-CD38 antibody and the PARPi are administered sequentially or separately.
54. A method of treating a disease in a subject in need thereof, comprising administering to the subject an anti-CD38 antibody and an adenosine receptor antagonist for a time sufficient to treat the disease.
55. The method of Embodiment 54, wherein the anti-CD38 antibody comprises:
   a) a heavy chain complementarity determining region 1 (HCDR1), HCDR2 and HCDR3 amino acid sequences of SEQ ID NOs: 6, 7 and 8, respectively; and
   b) a light chain complementarity determining region 1 (LCDR1), LCDR2 and LCDR3 amino acid sequences of SEQ ID NOs: 9, 10 and 11, respectively.
56. The method of Embodiment 54, wherein the anti-CD38 antibody comprises:
   a) a heavy chain variable region (VH) sequence of SEQ ID NO: 4; and
   b) a light chain variable region (VL) sequence of SEQ ID NO: 5.
57. The method of Embodiment 54, wherein the anti-CD38 antibody comprises a heavy chain sequence of SEQ ID NO: 12 and a light chain sequence of SEQ ID NO: 13.
58. The method of Embodiment 54, wherein the anti-CD38 antibody comprises:
   a) the heavy chain complementarity determining region 1 (HCDR1), HCDR2 and HCDR3 amino acid sequences of the heavy chain variable region (VH) of SEQ ID NO: 14 and the light chain complementarity determining region 1 (LCDR1), LCDR2, and LCDR3 amino acid sequences of the variable region (VL) of SEQ ID NO: 15;
   b) the HCDR1, HCDR2 and HCDR3 amino acid sequences of the VH of SEQ ID NO: 16 and the LCDR1, LCDR2, and LCDR3 amino acid sequences of the VL of SEQ ID NO: 17;
   c) the HCDR1, HCDR2 and HCDR3 amino acid sequences of the VH of SEQ ID NO: 18 and the LCDR1, LCDR2, and LCDR3 amino acid sequences of the VL of SEQ ID NO: 19; or
   d) the HCDR1, HCDR2 and HCDR3 amino acid sequences of the VH of SEQ ID NO: 20 and the LCDR1, LCDR2, and LCDR3 amino acid sequences of the VL of SEQ ID NO: 21.
59. The method of Embodiment 58, wherein the anti-CD38 antibody comprises the VH and VL sequences of:
   a) SEQ ID NOs: 14 and 15, respectively;
   b) SEQ ID NOs: 16 and 17, respectively;
   c) SEQ ID NOs: 18 and 19, respectively; or
   d) SEQ ID NOs: 20 and 21, respectively.
60. The method of any one of Embodiments 54-59, wherein the anti-CD38 antibody is of the IgG1, IgG2, IgG3 or IgG4 subtype.
61. The method of Embodiment 60, wherein the anti-CD38 antibody is of the IgG1 subtype.
62. The method of Embodiment 61, wherein the anti-CD38 antibody is of the IgG1/κ subtype.
63. The method of Embodiment 54, wherein the anti-CD38 antibody is daratumumab.
64. The method of any one of Embodiments 54-63, wherein the anti-CD38 antibody is administered intravenously.
65. The method of any one of Embodiments 54-63, wherein the anti-CD38 antibody is administered subcutaneously.
66. The method of Embodiment 65, wherein the anti-CD38 antibody is administered in a pharmaceutical composition comprising the anti-CD38 antibody and a hyaluronidase.
67. The method of Embodiment 66, wherein the hyaluronidase is rHuPH20 and has the amino acid sequence of SEQ ID NO: 22.
68. The method of any one of Embodiments 54-67, wherein the disease is cancer.
69. The method of Embodiment 68, wherein the cancer is a hematologic cancer.
70. The method of Embodiment 69, wherein the hematologic cancer is leukemia.
71. The method of Embodiment 70, wherein the leukemia is acute lymphoblastic leukemia (ALL), acute myeloid leukemia (AML), chronic lymphocytic leukemia (CLL), chronic myeloid leukemia (CML), hairy cell leukemia (HCL) or myelodysplastic syndromes (MDS).
72. The method of Embodiment 69, wherein the hematologic cancer is lymphoma.
73. The method of Embodiment 72, wherein the lymphoma is Hodgkin lymphoma.
74. The method of Embodiment 73, wherein the Hodgkin lymphoma is nodular sclerosis Hodgkin lymphoma (NSCHL), mixed cellularity classical Hodgkin lymphoma (MCcHL), lymphocyte-rich Hodgkin's disease (LRCHL) or lymphocyte-depleted Hodgkin's disease (LDHL).
75. The method of Embodiment 72, wherein the lymphoma is non-Hodgkin lymphoma (NHL).
76. The method of Embodiment 75, wherein the non-Hodgkin lymphoma is a B cell lymphoma.
77. The method of Embodiment 76, wherein the B cell lymphoma is diffuse large B-cell lymphoma (DLBCL), primary mediastinal B cell lymphoma (PMBCL), follicular lymphoma (FL), small lymphocytic lymphoma (SLL), marginal zone lymphoma (MZL), mantle cell lymphoma (MCL), Waldenström's macroglobulinemia (WMG) or Burkitt lymphoma (BL).
78. The method of Embodiment 75, wherein the non-Hodgkin lymphoma is a T cell lymphoma.
79. The method of Embodiment 78, wherein the T cell lymphoma is peripheral T-cell lymphoma (PTCL), anaplastic large cell lymphoma (ALCL), angioimmunoblastic T-cell lymphoma (AITL) or cutaneous T cell lymphoma.
80. The method of Embodiment 79, wherein the hematologic cancer is multiple myeloma.
81. The method of Embodiment 80, wherein the multiple myeloma is light chain multiple myeloma (LCMM), non-secretory multiple myeloma (NSMM), solitary plasmacytoma (SP), extramedullary plasmacytoma (EMP), monoclonal gammopathy of undetermined significance (MGUS), smoldering Multiple Myeloma (SMM), Immunoglobulin D multiple myeloma (IgD MM) or Immunoglobulin E (IgE) multiple myeloma.
82. The method of Embodiment 69, wherein the hematologic cancer is a CD38-positive hematological malignancy.
83. The method of Embodiment 82, wherein the CD38-positive hematological malignancy is multiple myeloma (MM), acute lymphoblastic leukemia (ALL), non-Hodgkin's lymphoma (NHL), diffuse large B-cell lymphoma (DLBCL), Burkitt's lymphoma (BL), follicular lymphoma (FL), mantle-cell lymphoma (MCL), acute myeloid leukemia (AML) or chronic lymphocytic leukemia (CLL).
84. The method of Embodiment 82, wherein the CD38-positive hematological malignancy is a plasma cell disease.
85. The method of Embodiment 84, wherein the plasma cell disease is light chain amyloidosis (AL), multiple myeloma (MM) or Waldenström's macroglobulinemia.
86. The method of Embodiment 85, wherein the plasma cell disease is MM.
87. The method of Embodiment 85, wherein the plasma cell disease is AL.
88. The method of Embodiment 68, wherein the cancer is a solid tumor.
89. The method of Embodiment 88, wherein the solid tumor is a tumor of the breast, lung, prostate, colon, bladder, ovary, kidney, stomach, colon, rectum, testes, head and/or neck, pancreas, brain or skin.
90. The method of Embodiment 88, wherein the solid tumor is bladder cancer, brain cancer, breast cancer, cervical cancer, colon cancer, colorectal cancer, fallopian tube cancer, gastric cancer, genitourinary cancer, head and neck cancer, liver cancer, lung cancer, melanoma, nasopharyngeal carcinoma (NPC), pancreatic cancer, prostate cancer, ovarian cancer, rectal cancer, renal cancer, skin cancer, stomach cancer, testicular cancer, thyroid cancer or urethral cancer.
91. The method of Embodiment 88, wherein the solid tumor is squamous non-small cell lung cancer (NSCLC), non-squamous NSCLC, lung adenocarcinoma, mesothelioma, kidney clear cell carcinoma, kidney papillary cell carcinoma, castration-resistant prostate cancer, squamous cell carcinoma of the head and neck, carcinomas of the esophagus, carcinomas of the gastrointestinal tract or endometriosis.
92. The method of any one of Embodiments 88-91, wherein the solid tumor is a metastatic lesion of the cancer.
93. The method of any one of Embodiments 54-67, wherein the disease is a neurological disorder.
94. The method of Embodiment 93, wherein the neurological disorder is acute spinal cord injury (SCI), Alzheimer's Disease (AD), amyotrophic lateral sclerosis (ALS), ataxia, Bell's palsy, a brain tumor, cerebral aneurysm, epilepsy, Guillain-Barré syndrome (GBS), hydrocephalus, a lumbar disk disease, meningitis, multiple sclerosis (MS), muscular dystrophy, a neurocutaneous syndrome, Parkinson's disease (PD), stroke, a cluster headache, a tension headache, a migraine headache, encephalitis, septicemia or myasthenia gravis (MG).
95. The method of Embodiment 94, wherein the neurological disorder is Alzheimer's Disease (AD) or multiple sclerosis (MS).
96. The method of any one of Embodiments 54-67, wherein the disease is a liver disease.
97. The method of Embodiment 96, wherein the liver disease is alagille syndrome (ALGS), autoimmune hepatitis (AIH), biliary atresia, cirrhosis, hemochromatosis, hepatitis, nonalcoholic fatty liver disease (NAFLD), primary biliary cholangitis (PBC), primary sclerosing cholangitis (PSC) or Wilson disease (WD).
98. The method of Embodiment 97, wherein the NAFLD is non-alcoholic steatohepatitis (NASH).
99. The method of any one of Embodiments 54-98, wherein the adenosine receptor antagonist is an A₁ receptor (A₁AR) antagonist, an A_{2A} receptor (A_{2A}AR) antagonist, an A₂B receptor (A_{2B}AR) antagonist or an A₃ receptor (A₃AR) antagonist.
100. The method of Embodiment 99, wherein the adenosine receptor antagonist is an A₁AR antagonist.
101. The method of Embodiment 100, wherein the adenosine receptor antagonist is BG 9719, DPCPX, FK453, FR194921, N-0861, rolofylline (KW 3902), tonapofylline (BG 9928) or WRC-0571.
102. The method of Embodiment 101, wherein the disease is heart failure, renal insufficiency, hepatic impairment, dementia or anxiety disorder.
103. The method of Embodiment 102, wherein the heart failure is acute heart failure.
104. The method of Embodiment 101, wherein:
   a) the adenosine receptor antagonist is BG 9719, and the disease is renal insufficiency or congestive heart failure;
   b) the adenosine receptor antagonist is FR194921, and the disease is dementia or anxiety disorder;
   c) the adenosine receptor antagonist is rolofylline (KW-3902), and the disease is heart failure or renal insufficiency; or
   d) the adenosine receptor antagonist is tonapofylline (BG 9928), and the disease is heart failure, renal insufficiency or hepatic impairment.
105. The method of Embodiment 104, wherein the heart failure is congestive heart failure.
106. The method of Embodiment 104, wherein the heart failure is acute heart failure.
107. The method of Embodiment 99, wherein the adenosine receptor antagonist is an A_{2A}AR antagonist.
108. The method of Embodiment 107, wherein the adenosine receptor antagonist is caffeine, 8-(-3-chlorostyryl)-caffeine (CSC), istradefylline (KW-6002), Preladenant (SCH 420814), Schering compound, SCH 58261, SCH 442416, SYN115, VER 6947, VER 7835 or ZM241, 385.
109. The method of Embodiment 108, wherein the disease is Parkinson's Disease (PD), restless legs syndrome, cerebral ischaemia or herniated lumbar disc.
110. The method of Embodiment 108, wherein:
   a) the adenosine receptor antagonist is caffeine, and the disease is Parkinson's Disease (PD);
   b) the adenosine receptor antagonist is istradefylline (KW-6002), and the disease is Parkinson's Disease (PD) or restless legs syndrome;
   c) the adenosine receptor antagonist is Preladenant (SCH 420814), and the disease is Parkinson's Disease (PD);
   d) the adenosine receptor antagonist is SCH 58261, and the disease is cerebral ischaemia;
   e) the adenosine receptor antagonist is SCH 442416, and the disease is Parkinson's Disease (PD);
   f) the adenosine receptor antagonist is SYN115, and the disease is Parkinson's Disease (PD); or
   g) the adenosine receptor antagonist is a compound of formula I, and the disease is herniated lumbar disc

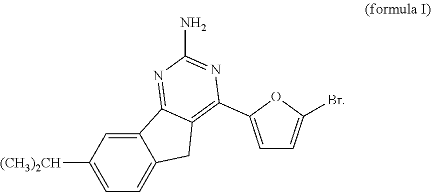

111. The method of Embodiment 99, wherein the adenosine receptor antagonist is an A_{2B}AR antagonist.
112. The method of Embodiment 111, wherein the adenosine receptor antagonist is MRE 2029-F20, MRS1754, OSIP-339391 or a compound of formula II

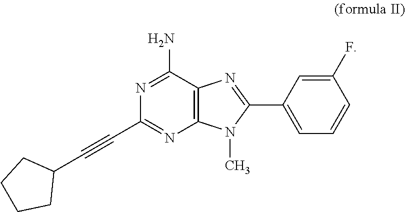 [SMILES: Cn1c(-c2cccc(F)c2)nc2c(N)nc(C#CC3CCCC3)nc21]

113. The method of Embodiment 99, wherein the adenosine receptor antagonist is an A₃AR antagonist.
114. The method of Embodiment 113, wherein the adenosine receptor antagonist is FA385, MRE 3008-F20, MRS1292, MRS1334, MRS1523, MRS3777, OT-7999, PSB-11, VUF5574 or a compound of formula III

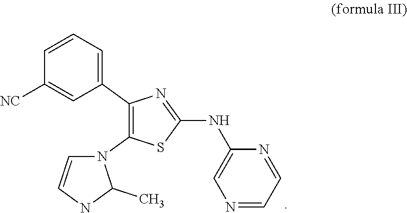 [SMILES: CC1NC=CN1c1sc(Nc2cnccn2)nc1-c1cccc(C#N)c1]

115. The method of any one of Embodiments 54-114, wherein the anti-CD38 antibody and the adenosine receptor antagonist are administered simultaneously.
116. The method of any one of Embodiments 54-114, wherein the anti-CD38 antibody and the adenosine receptor antagonist are administered sequentially or separately.
117. The method of any one of Embodiments 54-116, further comprising administering to the subject a poly ADP ribose polymerase inhibitor (PARPi) for a time sufficient to treat the disease.

### Examples

It is important to understand tissue- and age-specific effects of CD38 reduction in a mammalian model.

CD38 plays a critical role in NAD⁺ consumption. Extracellular NAD⁺ is broken down by CD38 to produce nicotinamide (NAM) or nicotinamide mononucleotide (NMN), which is further broken down to nicotinamide riboside (NR). NR enters cells through a nucleotide transporter and participates in intracellular NAD⁺ biogenesis. NR is converted to NMN, and NAM is converted to NMN. The pathways merge at the step of NMN formation, which is further converted to NAD⁺. Nicotinic acid (NA) is converted to NA mononucleotide (NAMN), NA adenine dinucleotide (NAAD), and then NAD⁺. NAD⁺ is also used as a cofactor of S-adenosylhomocysteine (SAH) hydrolase for the generation of intracellular adenosine. A net loss of NAD⁺ is associated with enzymatic reactions that take place during ADP-ribose formation (NAD⁺ glycohydrolase), polyADP-ribosylation (PARPs), and the de-acetylation of proteins (Sirtuins). See, e.g., Horenstein A L et al., Cells 4(3):520-37 (2015).

NAD⁺ is an essential co-enzyme and a central signaling molecule involved in maintaining redox homeostasis, efficient signal transduction, and mitochondrial metabolism. The extracellular conversion of NAD⁺ can vary significantly according to the tissue environment or pathological conditions (Horenstein et al., Cells. 4(3):520-37 (2015)). Accumulating evidence suggests that tumor cells exploit such a network for migrating and homing to protected areas and, even more importantly, for evading the immune response (Id.).

CD38 also plays a role in adenosine generation and signaling. In some cancers, NAD⁺ released by the salvage pathway is hydrolyzed to adenosine through the CD38-CD203a-CD73 pathway. Accumulated adenosine is further degraded to inosine in the presence of adenosine deaminase (ADA) through its association with CD26. See, e.g., Vijayan D et al., Nat. Rev. Cancer 17(12):709-24 (2017).

CD38 also mediates changes in NAD⁺ metabolism and generation of adenosine with age. It has been postulated that increased CD38 expression, with age, results in a decline in NAD⁺ and mitochondrial dysfunction, thereby affecting metabolism and brain and immune function. See, e.g., Camacho-Pereira J et al., Cell Metab. 23(6):1127-39 (2016). For example, CD38 regulates age-related NAD⁺ decline in liver and spleen. Id. The CD38-mediated pathway is also thought to underline adenosine generation in the bone marrow niche upon progression to multiple myeloma. Horenstein A L et al., Mol. Med. 22:694-704 (2016).

### Example 1. Generation, Validation and Characterization of CD38-KO Mice

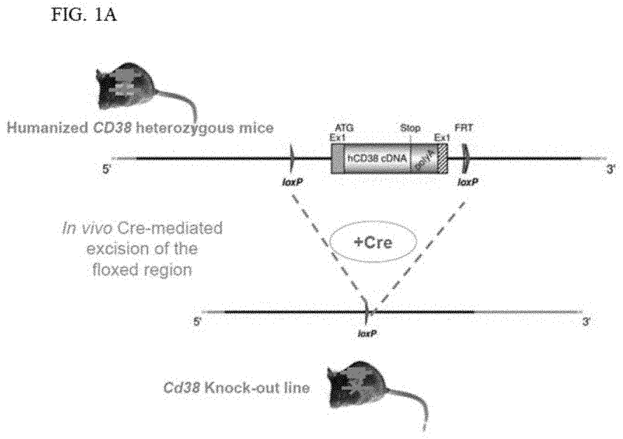

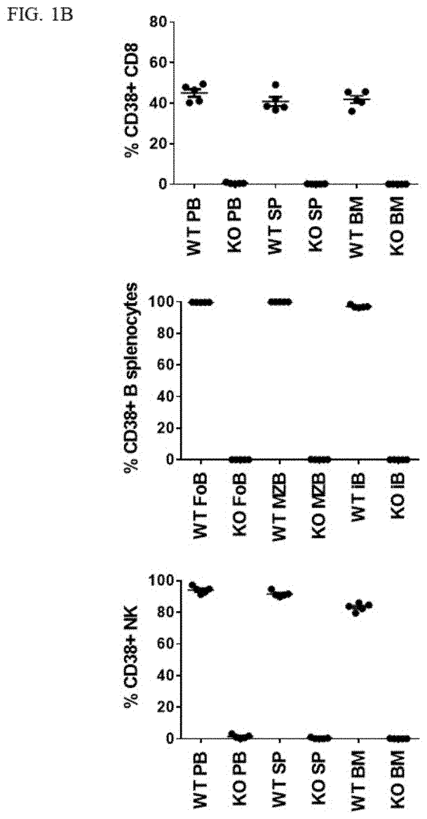

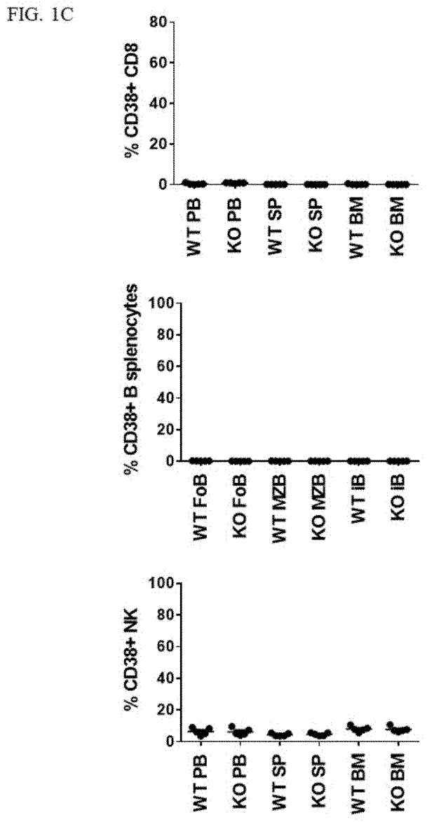

To generate CD38-KO C57BL/6N mice, mouse CD38 expression was disrupted by inserting human CD38 (hCD38), flanked by loxP sites, in frame with the start codon. The locus of inserted region is devoid of known regulatory elements to prevent disruption of mouse regulatory sequences. The transgene was under the control of the endogenous mouse promoter, allowing for the conservation of the murine CD38 expression pattern. The hCD38 transgene was subsequently deleted by Cre-mediated excision of the foxed region in vivo, by crossing the hCD38 transgenic mice with Cre-expressing mice (FIG. 1A). C57BL/6N wildtype and CD38-KO mice were bred at Charles River Laboratories (Wilmington, Mass.). FACS analysis was used for validating the CD38-KO line. Mouse CD38 was not detected on immune subsets of CD38-KO mice (FIG. 1B) and human CD38 was absent from B and NK cells of CD38-KO mice (FIG. 1C).

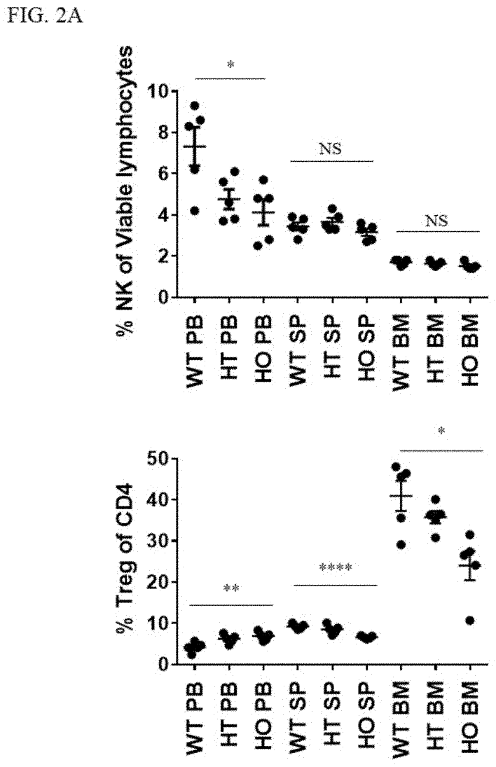

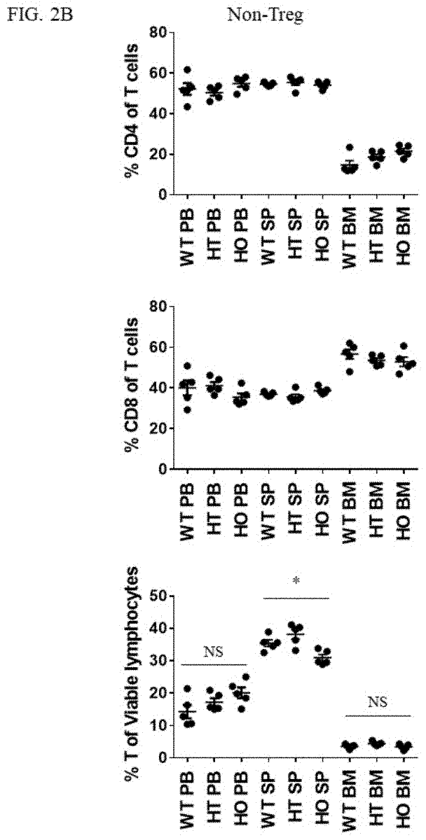

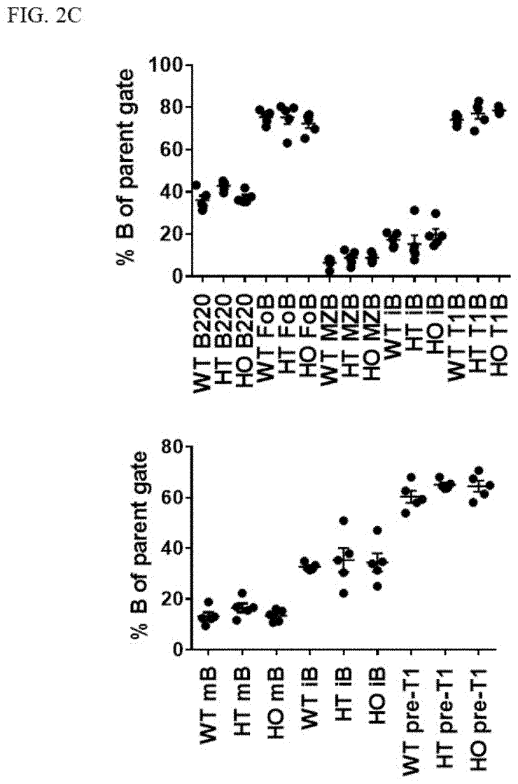

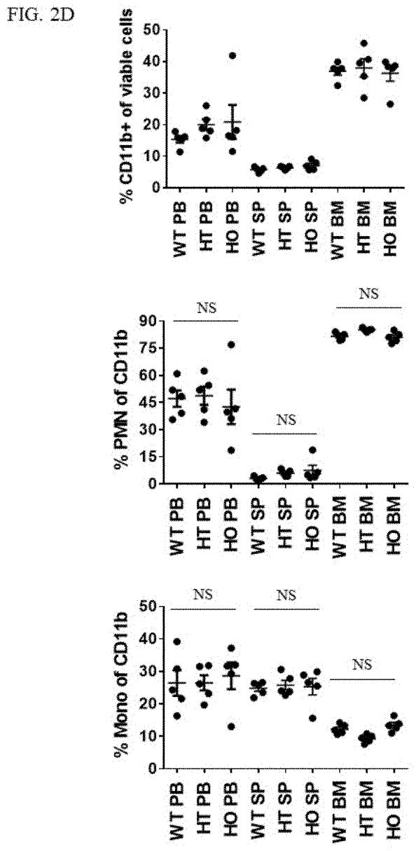

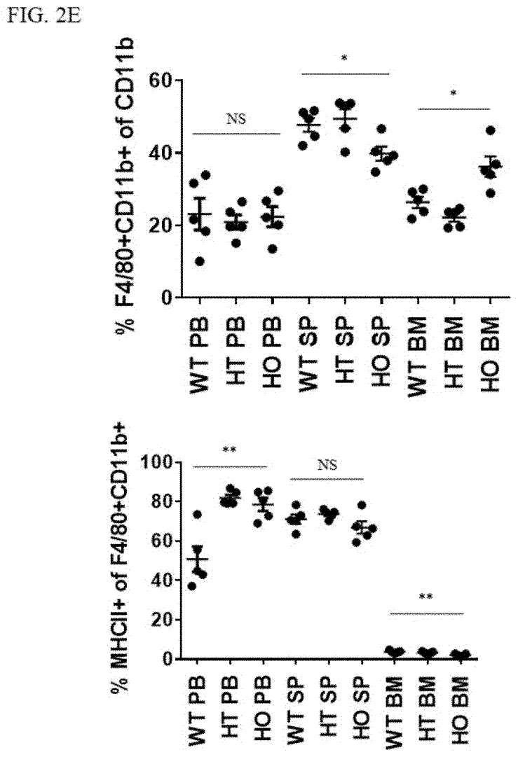

FACS analysis was also used for characterizing the CD38-KO line. Mature natural killer cells (NKs) and Regulatory T cells (Tregs) were modulated in CD38-KO mice. NKs were reduced in the peripheral blood (FIG. 2A). Tregs were reduced in the spleen and bone marrow but were increased in the peripheral blood (FIG. 2A). Except for Tregs, T cells were present at normal proportions in CD38-KO mice (FIG. 2B). These changes are consistent with observations in the hCD38-knockin line. Total T cell decrease was observed in spleen likely due to significant reduction of splenic CD4 Tregs. B cells proportions were normal in CD38-KO mice (FIG. 2C) while a decrease in FoB cells was observed in hCD38-knockin mice. The myeloid compartment was not affected in CD38-KO mice (FIG. 2D). Finally, macrophage populations in CD38-KO mice varied in different organs (FIG. 2E).

### Example 2. Quantification of NAD⁺, cADPR and Adenosine in CD38-WT and CD38-KO Mice

The CD38^{-/-} (CD38-KO) mouse model was used to investigate effects of therapeutic anti-CD38 antibodies on NAD⁺, adenosine and cADPR levels. Tissues were collected from young and old C57BL6 CD38-KO mice and age-matched CD38^{+/+} (CD38-wild-type (WT)) mice. Specifically, the six young CD38-KO mice included three females that were four to six weeks old, two females that were eight weeks old, and one male that was four to six weeks old. The four young CD38-WT mice included two females and two males that were four to six weeks old. The five old CD38-KO mice included one female and four males that were about six months old. The five old CD38-WT mice included one female and four males that were about six months old. Levels of NAD⁺ in flash frozen tissues were measured by liquid chromatography and mass spectrometry.

The following chemicals were used: acetonitrile (HPLC grade, EMD Millipore Burlington, Mass.), methanol (HPLC grade, EMD Millipore, Burlington, Mass.), formic acid (reagent grade, Honeywell Fluka, Charlotte, N.C.), trifluoroacetic acid (reagent grade, Thermo Fisher Scientific, Inc., Waltham, Mass.) and perchloric acid (certified ACS grade, Thermo Fisher Scientific, Inc., Waltham, Mass.).

A solution of 50:50 methanol/water was prepared by mixing equal volumes of methanol (HPLC grade, EMD Millipore, Burlington, Mass.) and nanopure water well. A solution of 0.1% formic acid in methanol/water (50/50) was prepared by transferring 1 volume (e.g., 1 mL) of formic acid (reagent grade, Honeywell Fluka, Charlotte, N.C.) into 500 volumes (e.g., 500 mL) of 50:50 methanol/water and mix well. A solution of 0.5 M perchloric acid (PCA) in water was prepared by mixing 39 volumes (e.g., 39 mL) of PCA (certified ACS grade, Thermo Fisher Scientific, Inc., Waltham, Mass.) and 1,000 volumes (e.g., 1,000 mL) of nanopure water well. The solution was cooled to ice-cold before use. A solution of 0.1% trifluoroacetic acid in water was prepared by mixing 1 volume (e.g., 1 mL) of trifluoroacetic acid (reagent grade, Thermo Fisher Scientific, Inc., Waltham, Mass.) to 1,000 volumes (e.g., 1,000 mL) of water well.

The following standards were used: adenosine (Sigma-Aldrich, St. Louis, Mo.), NAD⁺ (Sigma-Aldrich, St. Louis, Mo.), cADPR (Toronto Research Chemicals Inc., Ontario, Canada), NAD⁺-d3 (Toronto Research Chemicals Inc., Ontario, Canada), adenosine-13C (Toronto Research Chemicals Inc., Ontario, Canada) and AMP-15N5 (Toronto Research Chemicals Inc., Ontario, Canada).

Working standards were prepared in 0.1% formic acid (reagent grade, Honeywell Fluka, Charlotte, N.C.) in methanol/water (50/50) by dilution of the primary standard stock solution as shown in Table 2.

Primary internal standard stock solution was prepared at 1 mg/mL in methanol/water (50/50). Working internal solution was prepared in 0.1% formic acid in methanol/water (50/50) to contain the NAD⁺-d3, adenosine-13C5, and AMP-15N5 internal standards each at 2,500 ng/mL.

TABLE 2
Preparation of Working Standards

| Stock Used | Volume Spike | Final Volume | Analyte Concentration |
|---|---|---|---|
| (μg/mL) | (μL) | (mL) | (ng/mL) |
| 1000 | 0.100 | 10 | 10000 |
| 10 | 2.0 | 4 | 5000 |
| 1000 | 1.0 | 4 | 2500 |
| 10 | 0.5 | 5 | 1000 |
| 5 | 0.50 | 5 | 500 |
| 2.5 | 0.50 | 5 | 250 |
| 1.0 | 0.50 | 5 | 100 |
| 0.5 | 0.50 | 5 | 50 |
| 0.25 | 0.50 | 5 | 25 |
| 0.10 | 0.50 | 5 | 10 |

For QC preparation, three lots of control tissue samples (n=2 each lot) were included in each batch and measured for endogenous level of analytes for evaluation of reproducibility of the assay.

Brain, liver, lung, lymph nodes, right femur, spleen and whole blood were collected from CD38-WT and CD38-KO mice. Immediately after collection, the tissues were flash frozen using liquid nitrogen. The tissues were stored at −80° C. and shipped on dry ice.

Approximately 0.2 g or less frozen tissue was placed into a 2-mL homogenization vial pre-filled with mixed bead, on ice. 1.0 mL of ice-cold 0.5M perchloric acid in water was added. The sample was homogenized at 6,500 rpm, with two 20-second intervals. The homogenate was centrifuged at 14,000 rpm using a micro centrifuge.

An 0.1 mL aliquot of homogenate supernatant or standard was placed into a corresponding conical test tube. 0.1 mL of working internal standard, 2,500 ng/mL of adenosine-13C5, NAD⁺-d3 and cAMP-13C5 in 0.1% formic acid in methanol/water (50/50), was added. Each tube was vortexed and dried at 40° C. for 10 minutes under nitrogen to remove the organic. (The samples do not need to be completely dry after 10 minutes.) Each tube was reconstituted by adding 0.5 mL of 0.5 M perchloric acid. Each tube was vortexed followed by 5-minute centrifugation at 3,000 rpm. Approximately 0.2 mL supernatant was transferred into a HPLC vial and capped.

For Method 1, Analytical Conditions, HPLC used two pumps: PUMP A (Shimadzu LC-20AD) and PUMP B (Shimadzu LC-20AD). The mobile phase comprised A (0.1% trifluoracetic acid in water) and B (acetonitrile). The flow rate was 0.5 mL/min.

TABLE 3
GRADIENT

| Time | % A | % B |
|---|---|---|
| 0.0 | 100 | 0 |
| 2 | 75 | 25 |
| 2.1 | 5 | 95 |
| 3.0 | 5 | 95 |
| 3.1 | 100 | 0 |

A Shimadzu SIL-20AC autosampler was used. The injection volume was 10 μL (5-20 μL) and the stop time was 5.5 minutes. Methanol was used for needle wash. The temperature was 5° C. Imtakt Unison UK-100 analytical column (100×2 mm; I.D.: 3 μm; PN: UK024) was used. Shimadzu CTO-20AC column oven was used; and switch valve was not used. Temperature: not used.

The mass spectrometer used PE SCIEX API 5000 MS/MS #01 as the detector. Data acquisition was performed using PC MS-01.

TABLE 4

| INTERFACE | TIS |
|---|---|
| PROBE X-axis POSITION (X) | 0 |
| PROBE Y-axis POSITION (Y) | 5 |
| ACQUISITION TIME | 3.0 min |
| POLARITY | (x Positive Negative) |

TABLE 5
Ion Monitored

| Analyte | Q1 | Q3 |
|---|---|---|
| NAD⁺ | 664.3 | 136.0 |
| NAD⁺-d3 | 667.3 | 136.0 |
| Adenosine | 268.1 | 136.0 |
| Adenosine 13C5 | 273.1 | 136.0 |

For Method 2, Analytical Conditions, HPLC used two pumps: PUMP A (Shimadzu LC-20AD) and PUMP B (Shimadzu LC-20AD). The mobile phase comprised A (0.1% trifluoracetic acid in water) and B (acetonitrile). The flow rate was 0.5 mL/min.

TABLE 6
Gradient

| Time | % A | % B |
|---|---|---|
| 0.0 | 80 | 20 |
| 3.0 | 20 | 80 |
| 3.1 | 80 | 20 |

A Shimadzu SIL-20AC autosampler was used. The injection volume was 10 μL (5-20 μL) and the stop time was at 5.0 minutes. Methanol was used for needle wash. The temperature was 5° C. Thermo Hypercarb analytical column (50×3 mm; I.D.: 3 μm; PN: 35003-053030) was used. Shimadzu CTO-20AC column oven was used; and switch valve was not used. Temperature: not used.

The mass spectrometer used PE SCIEX API 5000 MS/MS #01 as the detector. Data acquisition was performed using PC MS-01.

TABLE 7

| INTERFACE | TIS |
|---|---|
| PROBE X-axis POSITION (X) | 0 |
| PROBE Y-axis POSITION (Y) | 5 |
| ACQUISITION TIME | 3.0 min |
| POLARITY | (x Positive Negative) |

TABLE 8
Ion Monitored

| Analyte | Q1 | Q3 |
|---|---|---|
| cADPR | 542.1 | 136.0 |
| AMP-15N5 | 335.2 | 136.0 |

Chromatogram peaks were integrated using an Analyst version 1.6.2 software package. A weighted (1/x² where x equals concentration) linear regression analysis was used. The peak area ratios of analyte to the internal standard versus the nominal concentration were plotted. The slope, intercept and the correlation coefficient were calculated. The unknown concentration (x) was then calculated with the following formula: x=(y−b)/m. Where y was the peak area ratio of unknown analyte to internal standard, b was the y intercept and m was the slope.

M180701.02 and M180701.03 were revised. M180701.02 was revised with updates which included use of API5000, change of column for Method 1, tweaks in gradient, and use of cAMP-13C5.

### Example 3. Genetic Disruption of CD38 Significantly Increased NAD⁺ Levels

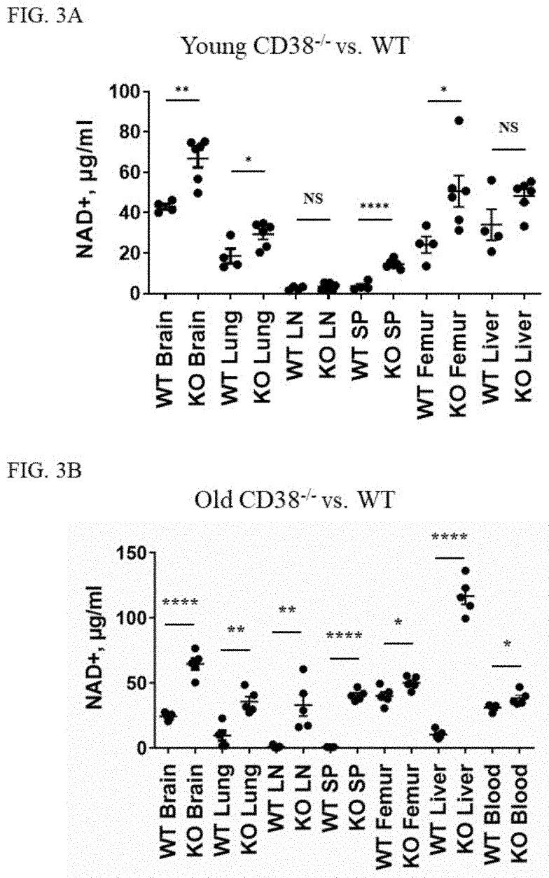

In young mice, genetic disruption of CD38 resulted in a significant increase of NAD⁺ levels in the brain, femur, lung and spleen (FIG. 3A and Table 9). In the brain, NAD⁺ levels were 43.00±1.39 μg/ml and 66.75±4.41 μg/ml in CD38-WT and CD38-KO, respectively (p≤0.01). In the femur, NAD⁺ levels were 24.10±4.11 μg/ml and 50.65±7.77 μg/ml in CD38-WT and CD38-KO, respectively (p≤0.05). In the lung, NAD⁺ levels were 18.52±3.62 μg/ml and 29.30±2.47 μg/ml in CD38-WT and CD38-KO, respectively (p≤0.05). In the spleen, NAD⁺ levels were 3.65±1.04 μg/ml and 14.55±0.87 μg/ml in CD38-WT and CD38-KO, respectively (p≤0.0001).

In young mice, genetic disruption of CD38 did not result in a significant change of NAD⁺ levels in the liver or lymph nodes (LN) (FIG. 3A and Table 9). In the liver, NAD⁺ levels were 34.01±7.67 μg/ml and 48.28±3.31 μg/ml in CD38-WT and CD38-KO, respectively. In the lymph nodes, NAD⁺ levels were 2.52±0.46 μg/ml and 3.40±0.68 μg/ml in CD38-WT and CD38-KO, respectively.

In old mice, genetic disruption of CD38 resulted in a significant increase of NAD⁺ levels in the blood, brain, femur, liver, lung, lymph nodes and spleen (FIG. 3B and Table 9). In the blood, NAD⁺ levels were 30.93±1.13 μg/ml and 38.21±2.38 μg/ml in CD38-WT and CD38-KO, respectively (p≤0.05). In the brain, NAD⁺ levels were 24.30±1.27 μg/ml and 64.57±4.28 μg/ml in CD38-WT and CD38-KO, respectively (p≤0.0001). In the femur, NAD⁺ levels were 40.00±3.12 μg/ml and 50.08±2.25 μg/ml in CD38-WT and CD38-KO, respectively (p≤0.05). In the liver, NAD⁺ levels were 10.34±1.51 μg/ml and 116.67±6.20 μg/ml in CD38-WT and CD38-KO, respectively (p≤0.0001). In the lung, NAD⁺ levels were 9.60±3.87 μg/ml and 35.51±3.94 μg/ml in CD38-WT and CD38-KO, respectively (p≤0.01). In the lymph nodes, NAD⁺ levels were 0.94±0.49 μg/ml and 32.93±8.34 μg/ml in CD38-WT and CD38-KO, respectively (p≤0.01). In the spleen, NAD⁺ levels were 0.68±0.05 μg/ml and 40.53±1.86 μg/ml in CD38-WT and CD38-KO, respectively (p≤0.0001).

The results from the old mice were also analyzed based on the weight of the tissue. In the brain, NAD⁺ levels were 97.18±5.08 μg/g and 258.27±17.11 μg/g in CD38-WT and CD38-KO, respectively (p≤0.0001). In the femur, NAD⁺ levels were 159.98±12.47 μg/g and 250.42±11.24 μg/g in CD38-WT and CD38-KO, respectively (p≤0.001). In the liver, NAD⁺ levels were 51.71±7.57 μg/g and 583.37±31.01 μg/g in CD38-WT and CD38-KO, respectively (p≤0.0001). In the lung, NAD⁺ levels were 30.19±10.93 μg/g and 177.54±19.71 μg/g in CD38-WT and CD38-KO, respectively (p≤0.001). In the lymph nodes, NAD⁺ levels were 4.69±2.46 μg/g and 164.66±41.72 μg/g in CD38-WT and CD38-KO, respectively (p≤0.01). In the spleen, NAD⁺ levels were 3.42±0.24 μg/g and 202.64±9.29 μg/g in CD38-WT and CD38-KO, respectively (p≤0.0001).

These data demonstrate that a decrease in CD38 expression resulted in significantly higher NAD⁺ levels in both young and old mice. The findings indicate that therapeutics that decreases CD38 expression may reduce NAD⁺ degradation and elicit resistance in patients. A significantly higher NAD⁺ level in the femur of CD38-KO mice, coupled with the knowledge that NAD⁺ inhibits apoptosis in MM cells by activating PARP and myeloma DNA repair pathways, suggest that NAD⁺ may mediate resistance mechanism to anti-CD38 antibody (e.g., daratumumab or HexaBody-CD38 (GEN3014)) treatment in patients (e.g., cancer such as multiple myeloma patients). Accordingly, PARPi likely benefits patients who are receiving or have received therapeutics that decrease CD38 expression.

### Example 4. CD38 Disruption-Mediated Increase in NAD⁺ was More Pronounced in Old Mice

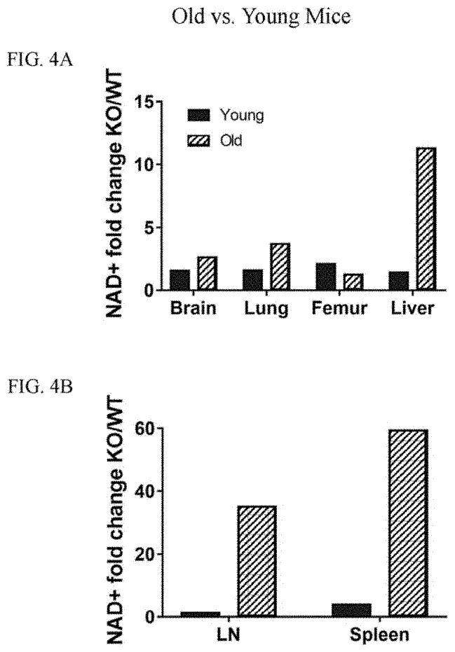

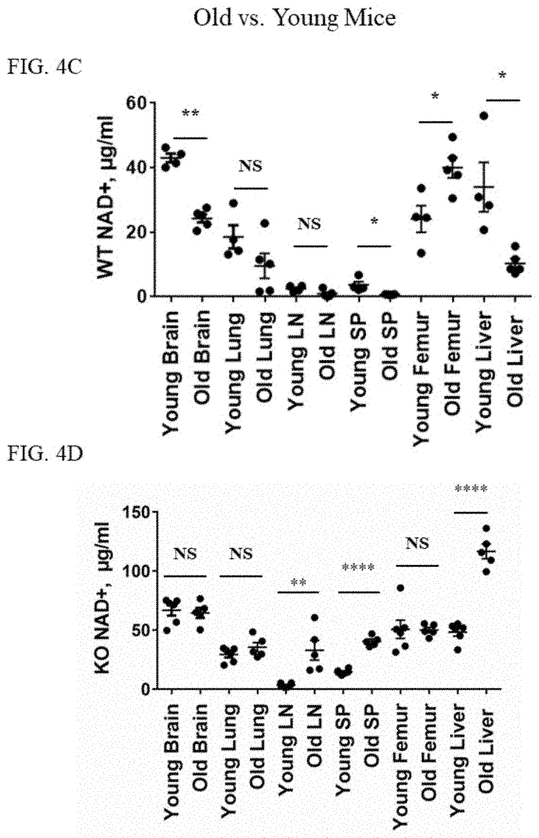

The increase in NAD⁺ levels associated with genetic disruption of CD38 was more profound in the old mice, relative to the young mice, in most tissues analyzed (FIGS. 4A-4B). In the brain, a 1.55-fold increase in NAD⁺ level was observed in the young mice, but a 2.66-fold increase was observed in the old mice. In the femur, a 2.10-fold increase in NAD⁺ level was observed in the young mice, and a 1.25-fold increase was observed in the old mice. In the liver, a 1.42-fold increase in NAD⁺ level was observed in the young mice, but a 11.28-fold increase was observed in the old mice. In the lung, a 1.58-fold increase in NAD⁺ level was observed in the young mice, but a 3.70-fold increase was observed in the old mice. In the lymph nodes, a 1.35-fold increase in NAD⁺ level was observed in the young mice, but a 35.03-fold increase was observed in the old mice. In the spleen, a 3.99-fold increase in NAD⁺ level was observed in the young mice, but a 59.60-fold increase was observed in the old mice.

In CD38-WT mice, the NAD⁺ level significantly decreased with age in the brain (43.00±1.39 μg/ml vs. 24.30±1.27 μg/ml, p≤0.01), liver (34.01±7.67 μg/ml vs. 10.34±1.51 μg/ml, p≤0.05) and spleen (3.65±1.04 μg/ml vs. 0.68±0.05 μg/ml, p≤0.05) (FIG. 4C and Table 11). The results are consistent with published observations that CD38 expression increases with age. The age-dependent decrease in NAD⁺, however, was not observed in the femur or lung of CD38-WT mice (FIG. 4C and Table 11).

An age-dependent decrease in NAD⁺ level was not observed in any tissue of the CD38-KO mice (FIG. 4D and Table 11). In the liver, lymph nodes and spleen, NAD⁺ levels significantly increased with age in CD38-KO mice (FIG. 4D and Table 11).

### Example 5. Effects of Genetic Disruption of CD38 on Adenosine Levels

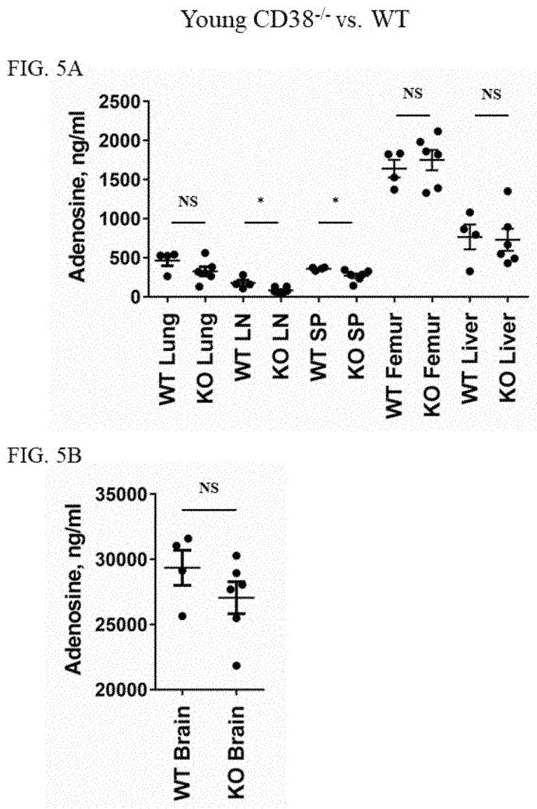

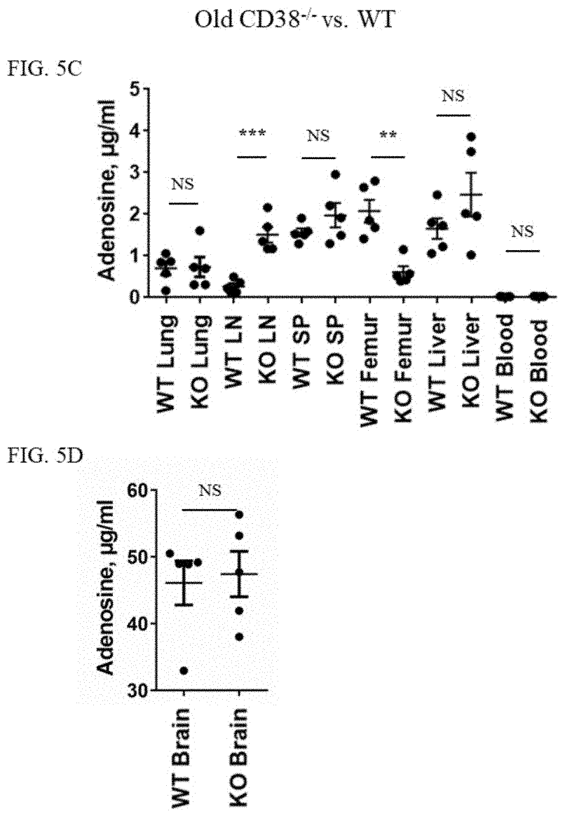

In young mice, genetic disruption of CD38 resulted in a significant decrease in adenosine levels in the lymph nodes and spleen (FIG. 5A and Table 10). In the lymph nodes, adenosine levels were 0.18±0.04 μg/ml and 0.08±0.02 μg/ml in CD38-WT and CD38-KO, respectively (p≤0.05). In the spleen, adenosine levels were 0.36±0.01 μg/ml and 0.27±0.03 μg/ml in CD38-WT and CD38-KO, respectively (p≤0.05).

In young mice, genetic disruption of CD38 did not result in a significant change in adenosine levels in the brain, femur, liver or lung (FIGS. 5A-5B and Table 10). In the brain, adenosine levels were 29.35±1.34 μg/ml and 27.05±1.22 μg/ml in CD38-WT and CD38-KO, respectively. In the femur, adenosine levels were 1.64±0.11 μg/ml and 1.75±0.13 μg/ml in CD38-WT and CD38-KO, respectively. In the liver, adenosine levels were 0.77±0.16 μg/ml and 0.73±0.14 μg/ml in CD38-WT and CD38-KO, respectively. In the lung, adenosine levels were 0.46±0.07 μg/ml and 0.33±0.06 μg/ml in CD38-WT and CD38-KO, respectively.

In old mice, genetic disruption of CD38 resulted in a significant decrease in adenosine levels in the femur (FIG. 5C and Table 10). In the femur, adenosine levels were 2.08±0.27 μg/ml and 0.61±0.14 μg/ml in CD38-WT and CD38-KO, respectively (p≤0.01).

In old mice, genetic disruption of CD38 resulted in a significant increase in adenosine levels in the lymph nodes (FIG. 5C and Table 10). In the lymph nodes, adenosine levels were 0.27±0.07 μg/ml and 1.51±0.19 μg/ml in CD38-WT and CD38-KO, respectively (p≤0.001).

In old mice, genetic disruption of CD38 did not result in a significant change in adenosine levels in the blood, brain, liver, lung or spleen (FIGS. 5C-5D and Table 10). In the blood, adenosine levels were 0.019±0.002 μg/ml and 0.027±0.003 μg/ml in CD38-WT and CD38-KO, respectively. In the brain, adenosine levels were 46.11±3.30 μg/ml and 47.45±3.40 μg/ml in CD38-WT and CD38-KO, respectively. In the liver, adenosine levels were 1.65±0.25 μg/ml and 2.47±0.53 μg/ml in CD38-WT and CD38-KO, respectively. In the lung, adenosine levels were 0.70±0.15 μg/ml and 0.73±0.24 μg/ml in CD38-WT and CD38-KO, respectively. In the spleen, adenosine levels were 1.56±0.10 μg/ml and 1.97±0.29 μg/ml in CD38-WT and CD38-KO, respectively.

These data demonstrate that a decrease in CD38 expression significantly increased adenosine levels in the lymph nodes in old mice. The findings indicate that therapeutics that decrease CD38 expression may increase adenosine production in human patient. Significantly higher adenosine levels in the lymph nodes, coupled with the knowledge that adenosine is an immunosuppressive metabolite produced at high levels within the tumor microenvironment, suggest that adenosine may also mediate resistance mechanism to anti-CD38 antibody (e.g., daratumumab or HexaBody-CD38 (GEN3014)) treatment in patients. Accordingly, adenosine receptor antagonists may benefit patients who are receiving or have received therapeutics that decrease CD38 expression.

### Example 6. Effects of CD38 Disruption on Adenosine Levels were Age-Dependent

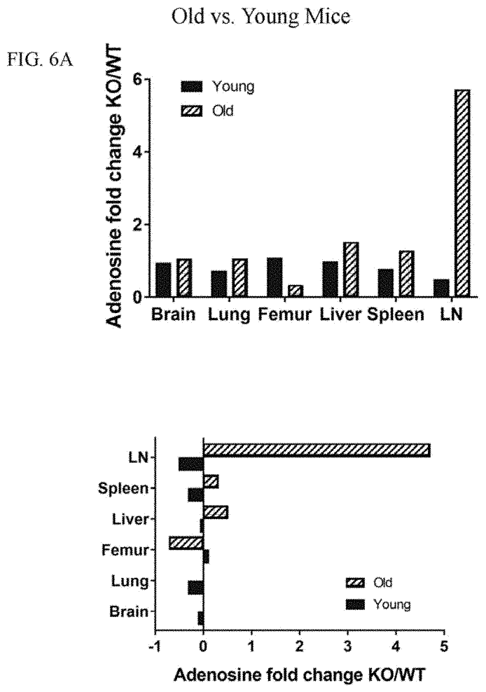

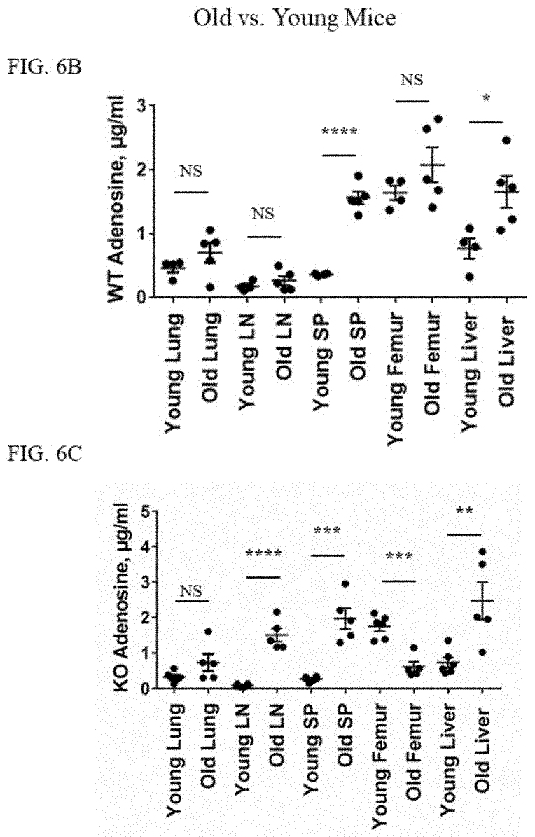

The change in adenosine level associated with genetic disruption of CD38 was age-dependent (FIGS. 6A-6C).

In the brain, an 8% decrease of adenosine level was observed in the young mice, and a 3% increase was observed in the old mice. In the femur, a 7% increase of adenosine level was observed in the young mice, and a 71% decrease was observed in the old mice. In the liver, a 5% decrease of adenosine level was observed in the young mice, and a 50% increase was observed in the old mice. In the lung, a 28% decrease of adenosine level was observed in the young mice, and a 4% increase was observed in the old mice. In the lymph nodes, a 56% decrease of adenosine level was observed in the young mice, but a 5.59-fold increase was observed in the old mice. In the spleen, a 25% decrease of adenosine level was observed in the young mice, but a 26% increase was observed in the old mice.

In CD38-WT mice, the adenosine level significantly increased with age in the liver and spleen (FIG. 6B and Table 12). The age-dependent increase, however, did not reach statistical significance in the femur, lung or lymph nodes in CD38-WT mice (FIG. 6B and Table 13).

In CD38-KO mice, the adenosine level significantly increased with age in the liver, lymph nodes and spleen (FIG. 6C and Table 12). The age-dependent increase, however, did not reach statistical significance in the lung in CD38-KO mice (FIG. 6C and Table 12). In CD38-KO mice, the adenosine level significantly decreased with age in the femur (FIG. 6C and Table 12).

### Example 7. Genetic Disruption of CD38 Resulted in Decrease of cADPR Levels in Young Mice

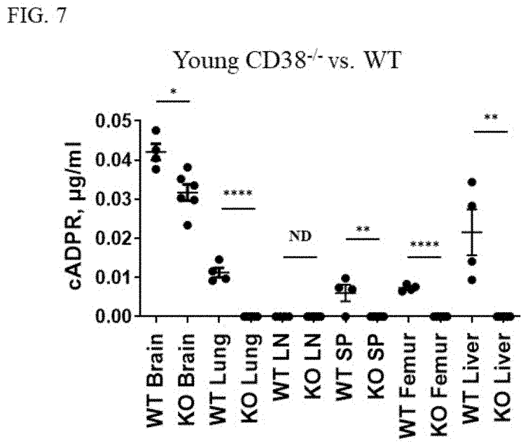

In young mice, genetic disruption of CD38 resulted in a significant decrease in cADPR levels in the brain, femur, liver, lung, and spleen (FIG. 7 and Table 13). In the brain, cADPR levels were 42.10±2.09 ng/ml and 31.73±2.10 ng/ml in CD38-WT and CD38-KO, respectively (p≤0.05). In the femur, cADPR levels were 7.33±0.40 ng/ml and undetectable in CD38-WT and CD38-KO, respectively (p≤0.0001). In the liver, cADPR levels were 21.55±5.87 ng/ml and undetectable in CD38-WT and CD38-KO, respectively (p≤0.01). In the lung, cADPR levels were 11.28±1.22 ng/ml and undetectable in CD38-WT and CD38-KO, respectively (p≤0.0001). In the spleen, cADPR levels were 6.05±2.11 ng/ml and undetectable in CD38-WT and CD38-KO, respectively (p≤0.01).

In young mice, cADPR levels were undetectable in the lymph nodes of CD38-WT and CD38-KO (FIG. 7 and Table 13).

Because cADPR is a downstream intermediate of NAD⁺ metabolism, a decrease in levels of cADPR in CD38-KO mice is consistent with an increase in levels of NAD⁺ in the knockout mice. Thus, the data provide further support that PARPi likely benefits patients who are receiving or have received therapeutics that decrease CD38 expression.

### Example 8. Assessment of Anti-CD38 Surrogate Immuno-Modulatory Properties in MC-38 Model

Experiments were designed to determine the effects of CD38 modulation on NAD⁺ metabolism (levels of NAD⁺, cADPR and adenosine) by removing cell surface CD38 using an anti-CD38 daratumumab surrogate.

Daratumumab does not bind mouse CD38. To obtain an anti-mouse CD38 surrogate monoclonal antibody, anti-CD38 NIMR5 mouse IgG2a antibody was generated by appending a sequence from the NIMR5 clone to an “active” mouse Fc, IgG2a (TeneoBio, Newark, Calif.). Mouse IgG2a is considered similar to human IgG1 that constitutes the Fc region of daratumumab. To obtain a “silent” anti-CD38 mouse surrogate, anti-CD38 NIMR5 mouse IgG2σ antibody was generated, in-house (Janssen Biologics (JBIO), Janssen Research and Development, L.L.C., Spring House, Pa.), by appending the sequence obtained from the NIMR5 clone to a “silent” mouse Fc, IgG2σ. The “silent” mouse Fc does not bind the Fc receptors on effector cells (e.g., NK cells and monocytes).

On Day 0, 0.5×10⁶ MC-38 murine colon adenocarcinoma cells were injected subcutaneously into the right hind flank of C57BL/6 or CD38-KO female mice.

On Day 7 post tumor implantation, mice were randomized into treatment groups, and the mean tumor volumes were approximately 52-72 mm³ by caliper measurement (Table 14). C57BL/6N mice were administered with isotype control (anti-mouse IgG2a) (Group 1), anti-CD38 mouse surrogate (Group 2), or “silent” anti-CD38 mouse surrogate (Groups 3 and 4) at a dose of 30 mg/kg (or 10 mg/kg in Group 3), every 3-4 days (q3d or q4d). CD38 KO mice received intraperitoneal (IP) treatment of isotype control (anti-mouse IgG2a) (Group 5) at 30 mg/kg, every 3-4 days (q3d or q4d). Each group received initial treatment at the indicated dose via IP injection on Day 7.

A total of three doses was administered, twice weekly via IP. Additionally, non-treated C57BL/6N control mice were randomized to study for immune phenotyping.

On Day 15, twenty-four hours after the third dose, terminal samplings of fresh spleen, tumor, draining lymph nodes, bone marrow, corresponding bone and intact femur were snap frozen to evaluate CD38 enzymatic activity by liquid chromatography mass spectrometry in response to anti-CD38 surrogate treatment. Metabolites analyses (NAD⁺, cADPR, adenosine) were performed as before, with exception of bone marrow that was eluted with 3 ml of RPMI 1640 medium. BMA was vortexed into a single cell suspension, and 100 μl was used for metabolites analysis. Raw values were plotted for 100 μl that were used for analysis.

In Groups 1 and 2, one fourth of the spleens, one fourth of the tumors, draining lymph nodes (DLN), and bone marrows were obtained by flushing the right femurs, and the right femurs and intact left femurs were snap frozen for measurements of CD38 metabolites levels. In Group 3, the entire spleens and entire tumors were placed into media for flow cytometry to monitor a loss of CD38 from immune and tumor cells. In Group 4, one fourth of the tumors were snap frozen for measurements of CD38 metabolites levels; and all of the spleens were placed into media for flow cytometry to monitor a loss of CD38 from immune and tumor cells. In Group 5, one half of the tumors, DLN, bone marrows obtained by flushing the right femurs, and the right femurs and intact left femurs were snap frozen for measurements of adenosine levels.

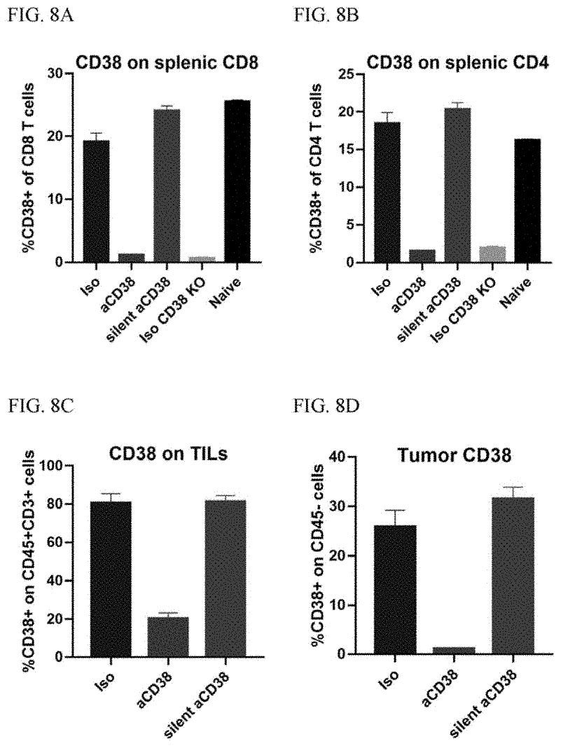

The effects of the anti-CD38 NIMR5 mouse IgG2a antibody on cell surface CD38 were investigated using flow cytometry with a monoclonal antibody (C38B680.004) labeled with allophycocyanin (APC). C38B680.004 was generated in-house and was confirmed to be non-competing with the NIMR5 clone. The spleens of naïve WT mice and CD38-KO tumor bearing mice were used to generate a positive control and a negative control, respectively. Treatment with the anti-CD38 NIMR5 mouse IgG2a antibody efficiently removed CD38 from splenic CD8 T cells (FIG. 8A), splenic CD4 T cells (FIG. 8B), tumor infiltrating T cells (TILs, FIG. 8C) and tumor cells (FIG. 8D). Active Fc (mouse IgG2a) was required for the removal of CD38 by the anti-CD38 NIMR5 mouse IgG2a surrogate mAb, because no removal was detected when a silent Fc (anti-CD38 NIMR5 mouse IgG2σ mAb) was used (FIGS. 8A-8D).

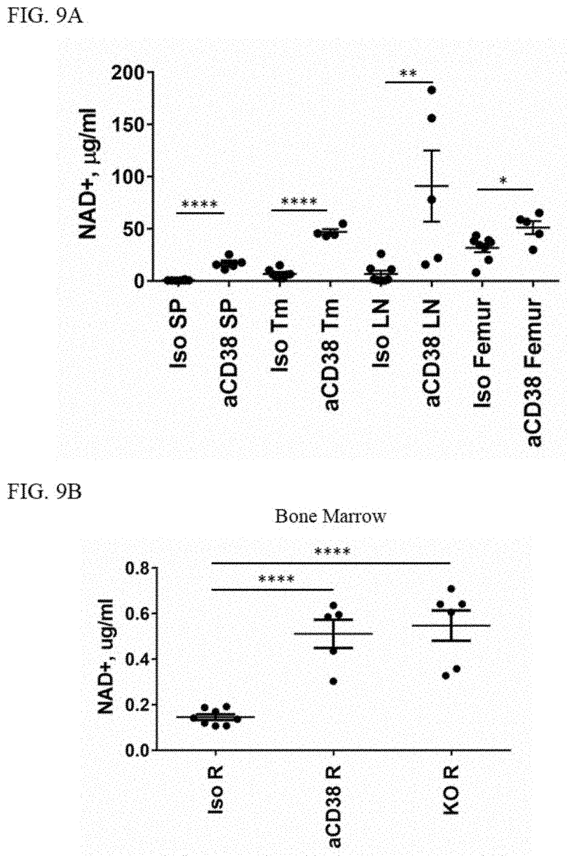

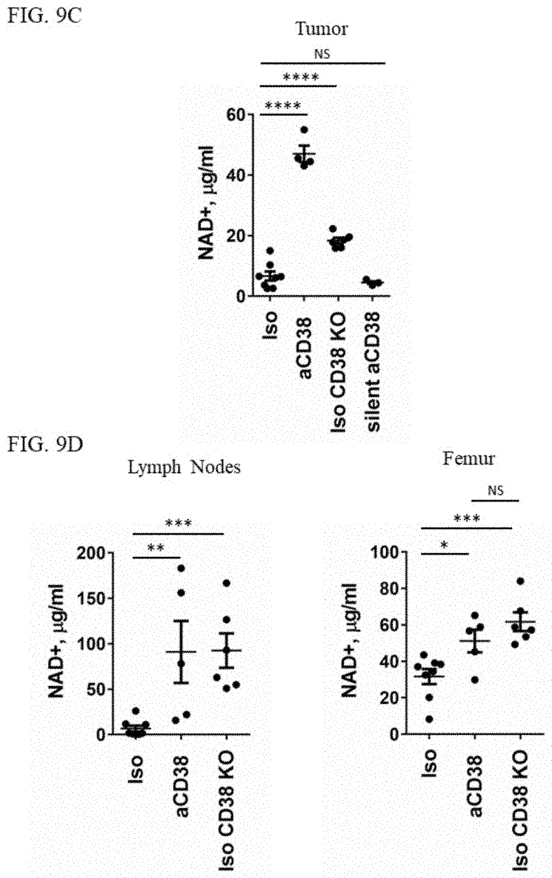

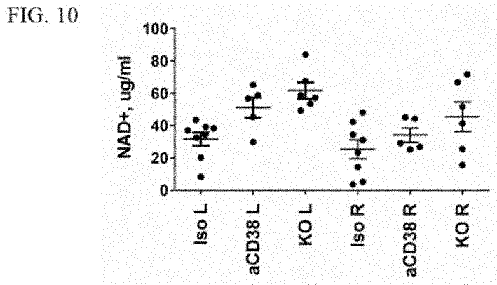

Treatment with an anti-CD38 NIMR5 mouse IgG2a antibody significantly increased NAD⁺ levels in the bone marrow, femur, lymph nodes, spleen and tumor (FIGS. 9A-9D). A 4-fold increase of NAD⁺ was observed in the bone marrow (FIG. 9B), compared to a 1.3-fold increase in corresponding bone or a 1.6-fold in intact femur (FIG. 9A), suggesting that the bone marrow (BMA) was the main site where metabolic changes occur upon treatment with an anti-CD38 surrogate monoclonal antibody. NAD⁺ levels were compared between the intact femur (L, left) and bone without BMA (R, right) (FIG. 10). Lower NAD⁺ was detected in the empty bone tissue. Although the same trend was observed in the bone and intact femur, likely the main differences occurred in the BMA.

Active Fc (mouse IgG2a) was required for the increase of NAD⁺ levels mediated by the anti-CD38 NIMR5 mouse IgG2a surrogate mAb NIMR-5. No increase was detected when a silent Fc (anti-CD38 NIMR5 mouse IgG2σ) was used (FIG. 9C). Thus, the modulation of NAD⁺ levels correlated with the removal of CD38 from the cell surface (FIGS. 8A-8D), and an active Fc is required for both processes.

In femur and lymph nodes, both anti-CD38 NIMR5 mouse IgG2a antibody treatments and genetic disruption of CD38 resulted in increase in NAD⁺ levels of similar magnitude (FIG. 9D). A weaker NAD⁺ accumulation in tumors of CD38-KO mice (FIG. 9C) was likely due to CD38 expression in tumor cells, suggesting that CD38-WT tumors consume the largest amount of NAD⁺ compared to the immune cells.

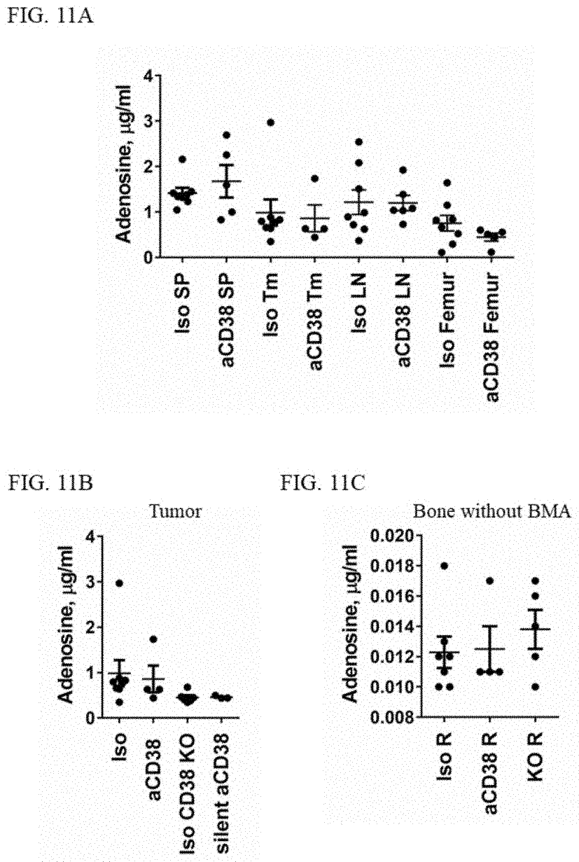

Treatment with the anti-CD38 NIMR5 mouse IgG2a antibody did not change adenosine level in the femur, lymph nodes, spleen or tumor in young mice (FIGS. 11A-11B), consistent with findings in young CD38-KO mice. Adenosine values, barely above the lower limit of quantitation due to BMA dilution during extraction, were deemed uninterpretable (FIG. 11C). Values below the lower limit of quantitation were not included in the analysis.

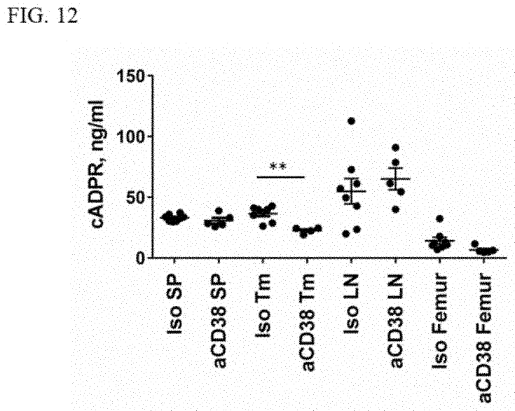

Finally, treatment with the anti-CD38 NIMR5 mouse IgG2a antibody decreased cADPR in the tumor (p<0.01) (FIG. 12). A decrease in cADPR, which did not reach statistical significance, also observed in the femur and spleen (FIG. 12).

These data directly demonstrate that therapeutics that decrease CD38 expression reduce NAD⁺ metabolism in tissues and tumors of mice, providing further support that NAD⁺ may mediate a resistance mechanism to anti-CD38 antibody (e.g., daratumumab or HexaBody-CD38 (GEN3014)) treatment in patients (e.g., multiple myeloma patients). Accordingly, PARPi likely benefits patients who are receiving or have received therapeutics that decrease CD38 expression.

TABLE 9
Effects of Genetic Disruption of CD38 on NAD⁺ Levels

| | NAD⁺ (μg/ml) | | | | NAD⁺ (μg/ml) | |
|---|---|---|---|---|---|---|
| Tissue | Young WT | Young KO | Old WT | Old KO | Old WT | Old KO |
| Blood | | | 30.22 | 35.72 | | |
| | | | 32.94 | 33.98 | | |
| | | | 26.81 | 39.77 | | |
| | | | 32.23 | 34.75 | | |
| | | | 32.45 | 46.84 | | |
| | | | 30.93 ± 1.13 | 38.21 ± 2.38 | | |
| Brain | 44.22 | 75.17 | 25.26 | 76.59 | 101.03 | 306.35 |
| | 41.45 | 74.69 | 25.76 | 62.55 | 103.05 | 250.18 |
| | 46.25 | 71.47 | 22.45 | 50.32 | 89.80 | 201.28 |
| | 40.07 | 72.80 | 27.59 | 68.31 | 110.35 | 273.25 |
| | | 49.72 | 20.42 | 65.07 | 81.69 | 260.27 |
| | | 56.68 | | | | |
| | 43.00 ± 1.39 | 66.75 ± 4.41 | 24.30 ± 1.27 | 64.57 ± 4.28 | 97.18 ± 5.08 | 258.27 ± 17.11 |
| Femur | 13.51 | 47.72 | 39.27 | 55.48 | 157.07 | 277.39 |
| | 24.55 | 52.08 | 42.98 | 54.48 | 171.93 | 272.39 |
| | 33.61 | 36.43 | 49.48 | 43.11 | 197.92 | 215.55 |
| | 24.75 | 85.63 | 30.51 | 48.46 | 122.04 | 242.28 |
| | | 31.33 | 37.74 | 48.90 | 150.94 | 244.49 |
| | | 50.69 | | | | |
| | 24.10 ± 4.11 | 50.65 ± 7.77 | 40.00 ± 3.12 | 50.08 ± 2.25 | 159.98 ± 12.47 | 250.42 ± 11.24 |
| Liver | 28.34 | 53.27 | 11.61 | 109.22 | 58.03 | 546.08 |
| | 30.86 | 55.33 | 15.68 | 99.48 | 78.40 | 497.41 |
| | 20.74 | 33.29 | 8.63 | 136.14 | 43.14 | 680.69 |
| | 56.09 | 51.88 | 7.24 | 115.66 | 36.22 | 578.31 |
| | | 50.72 | 8.56 | 122.88 | 42.79 | 614.38 |
| | | 45.18 | | | | |
| | 34.01 ± 7.67 | 48.28 ± 3.31 | 10.34 ± 1.51 | 116.67 ± 6.20 | 51.71 ± 7.57 | 583.37 ± 31.01 |
| Lung | 17.71 | 23.22 | 11.54 | 40.52 | 34.62 | 202.59 |
| | 28.99 | 32.88 | 10.18 | 31.69 | 30.54 | 158.47 |
| | 14.25 | 34.03 | 22.75 | 29.64 | 68.25 | 148.22 |
| | 13.14 | 34.68 | 1.86 | 27.21 | 9.30 | 136.06 |
| | | 30.60 | 1.65 | 48.47 | 8.23 | 242.36 |
| | | 20.37 | | | | |
| | 18.52 ± 3.62 | 29.30 ± 2.47 | 9.60 ± 3.87 | 35.51 ± 3.94 | 30.19 ± 10.93 | 177.54 ± 19.71 |
| Lymph | 1.49 | 2.51 | 0.45 | 28.99 | 2.27 | 144.95 |
| Nodes | 3.27 | 5.37 | 0.31 | 17.15 | 1.55 | 85.73 |
| | 3.34 | 5.23 | 2.79 | 60.52 | 13.96 | 302.62 |
| | 1.99 | 3.86 | 0.06 | 16.04 | 0.31 | 80.22 |
| | | 1.81 | 1.07 | 41.96 | 5.35 | 209.78 |
| | | 1.61 | | | | |
| | 2.52 ± 0.46 | 3.40 ± 0.68 | 0.94 ± 0.49 | 32.93 ± 8.34 | 4.69 ± 2.46 | 164.66 ± 41.72 |
| Spleen | 3.03 | 15.43 | 0.53 | 40.12 | 2.64 | 200.62 |
| | 6.72 | 18.08 | 0.76 | 41.86 | 3.80 | 209.29 |
| | 2.73 | 13.39 | 0.62 | 35.99 | 3.11 | 179.95 |
| | 2.13 | 14.04 | 0.75 | 37.86 | 3.75 | 189.30 |
| | | 14.57 | 0.76 | 46.81 | 3.81 | 234.04 |
| | | 11.77 | | | | |
| | 3.65 ± 1.04 | 14.55 ± 0.87 | 0.68 ± 0.05 | 40.53 ± 1.86 | 3.42 ± 0.24 | 202.64 ± 9.29 |

TABLE 10
Effects of Genetic Disruption of CD38 on Adenosine Levels

| | Adenosine (μg/ml) | | | |
|---|---|---|---|---|
| Tissue | Young WT | Young KO | Old WT | Old KO |
| Blood | | | 0.012 | 0.022 |
| | | | 0.019 | 0.037 |
| | | | 0.020 | 0.027 |
| | | | 0.025 | 0.020 |
| | | | 0.021 | 0.028 |
| | | | 0.019 ± 0.002 | 0.027 ± 0.003 |
| Brain | 31.04 | 28.95 | 48.90 | 56.35 |
| | 29.13 | 28.06 | 32.96 | 53.18 |
| | 25.64 | 27.69 | 50.53 | 41.95 |
| | 31.59 | 30.29 | 48.95 | 47.71 |
| | | 21.85 | 49.20 | 38.04 |
| | | 25.49 | | |
| | 29.35 ± 1.34 | 27.05 ± 1.22 | 46.11 ± 3.30 | 47.45 ± 3.40 |
| Femur | 1.83 | 1.39 | 2.64 | 1.15 |
| | 1.82 | 1.82 | 2.80 | 0.42 |
| | 1.53 | 1.98 | 1.68 | 0.52 |
| | 1.37 | 1.86 | 1.85 | 0.57 |
| | | 1.33 | 1.41 | 0.40 |
| | | 2.12 | | |
| | 1.64 ± 0.11 | 1.75 ± 0.13 | 2.08 ± 0.27 | 0.61 ± 0.14 |
| Liver | 1.08 | 0.89 | 1.80 | 3.86 |
| | 0.79 | 1.35 | 2.46 | 2.02 |
| | 0.33 | 0.49 | 1.06 | 1.02 |
| | 0.87 | 0.67 | 1.22 | 3.50 |
| | | 0.43 | 1.73 | 1.95 |
| | | 0.55 | | |
| | 0.77 ± 0.16 | 0.73 ± 0.14 | 1.65 ± 0.25 | 2.47 ± 0.53 |
| Lung | 0.26 | 0.26 | 0.86 | 0.73 |
| | 0.54 | 0.38 | 1.06 | 0.31 |
| | 0.53 | 0.56 | 0.84 | 0.70 |
| | 0.52 | 0.30 | 0.58 | 0.31 |
| | | 0.32 | 0.16 | 1.61 |
| | | 0.13 | | |
| | 0.46 ± 0.07 | 0.33 ± 0.06 | 0.70 ± 0.15 | 0.73 ± 0.24 |
| Lymph | 0.16 | 0.08 | 0.22 | 1.35 |
| Nodes | 0.16 | 0.13 | 0.36 | 2.16 |
| | 0.28 | 0.13 | 0.13 | 1.70 |
| | 0.11 | 0.05 | 0.50 | 1.17 |
| | | 0.07 | 0.12 | 1.17 |
| | | 0.04 | | |
| | 0.18 ± 0.04 | 0.08 ± 0.02 | 0.27 ± 0.07 | 1.51 ± 0.19 |
| Spleen | 0.36 | 0.28 | 1.29 | 1.50 |
| | 0.37 | 0.23 | 1.91 | 1.92 |
| | 0.33 | 0.34 | 1.51 | 2.21 |
| | 0.38 | 0.14 | 1.59 | 2.96 |
| | | 0.28 | 1.52 | 1.29 |
| | | 0.33 | | |
| | 0.36 ± 0.01 | 0.27 ± 0.03 | 1.56 ± 0.10 | 1.97 ± 0.29 |

TABLE 11
Effects of Age on NAD⁺ Levels

| | NAD⁺ Fold | | | | | |
|---|---|---|---|---|---|---|
| | Change KO/WT | | NAD⁺ (μg/ml) in WT | | NAD⁺ (μg/ml) in KO | |
| Tissue | Young | Old | Young | Old | Young | Old |
| Brain | 1.55 | 2.66 | 43.00 ± 1.39 | 24.30 ± 1.27 | 66.75 ± 4.41 | 64.57 ± 4.28 |
| Femur | 2.10 | 1.25 | 24.10 ± 4.11 | 40.00 ± 3.12 | 50.65 ± 7.77 | 50.08 ± 2.25 |
| Liver | 1.42 | 11.28 | 34.01 ± 7.67 | 10.34 ± 1.51 | 48.28 ± 3.31 | 116.67 ± 6.20 |
| Lung | 1.58 | 3.70 | 18.52 ± 3.62 | 9.60 ± 3.87 | 29.30 ± 2.47 | 35.51 ± 3.94 |
| Lymph Nodes | 1.35 | 35.03 | 2.52 ± 0.46 | 0.94 ± 0.49 | 3.40 ± 0.68 | 32.93 ± 8.34 |
| Spleen | 3.99 | 59.60 | 3.65 ± 1.04 | 0.68 ± 0.05 | 14.55 ± 0.87 | 40.53 ± 1.86 |

TABLE 12
Effects of Age on Adenosine Levels

| | Adenosine Fold | | | | | |
|---|---|---|---|---|---|---|
| | Change KO/WT | | Adenosine (μg/ml) in WT | | Adenosine (μg/ml) in KO | |
| Tissue | Young | Old | Young | Old | Young | Old |
| Brain | 0.92 | 1.03 | 29.35 ± 1.34 | 46.11 ± 3.30 | 27.05 ± 1.22 | 47.45 ± 3.40 |
| Femur | 1.07 | 0.29 | 1.64 ± 0.11 | 2.08 ± 0.27 | 1.75 ± 0.13 | 0.61 ± 0.14 |
| Liver | 0.95 | 1.50 | 0.77 ± 0.16 | 1.65 ± 0.25 | 0.73 ± 0.14 | 2.47 ± 0.53 |
| Lung | 0.72 | 1.04 | 0.46 ± 0.07 | 0.70 ± 0.15 | 0.33 ± 0.06 | 0.73 ± 0.24 |
| Lymph Nodes | 0.44 | 5.59 | 0.18 ± 0.04 | 0.27 ± 0.07 | 0.08 ± 0.02 | 1.51 ± 0.19 |
| Spleen | 0.75 | 1.26 | 0.36 ± 0.01 | 1.56 ± 0.10 | 0.27 ± 0.03 | 1.97 ± 0.29 |

TABLE 13
Effects of Genetic Disruption of CD38 on cADPR Levels

| | cADPR (μg/ml) | |
|---|---|---|
| Tissue | Young WT | Young KO |
| Brain | 37.7 | 33.5 |
| | 47.6 | 35.2 |
| | 40.5 | 30.3 |
| | 42.6 | 38.2 |
| | | 23.4 |
| | | 29.8 |
| | 42.10 ± 2.09 | 31.73 ± 2.10 |
| Femur | 6.5 | 0 |
| | 7.6 | 0 |
| | 8.3 | 0 |
| | 6.9 | 0 |
| | | 0 |
| | | 0 |
| | 7.33 ± 0.40 | 0 ± 0 |
| Liver | 28.3 | 0 |
| | 14.1 | 0 |
| | 9.4 | 0 |
| | 34.4 | 0 |
| | | 0 |
| | | 0 |
| | 21.55 ± 5.87 | 0 ± 0 |
| Lung | 9.2 | 0 |
| | 14.6 | 0 |
| | 11.6 | 0 |
| | 9.7 | 0 |
| | | 0 |
| | | 0 |
| | 11.28 ± 1.22 | 0 ± 0 |
| Lymph Nodes | 0 | 0 |
| | 0 | 0 |
| | 0 | 0 |
| | 0 | 0 |
| | | 0 |
| | | 0 |
| | 0 ± 0 | 0 ± 0 |
| Spleen | 0 | 0 |
| | 7.4 | 0 |
| | 7 | 0 |
| | 9.8 | 0 |
| | | 0 |
| | | 0 |
| | 6.05 ± 2.11 | 0 ± 0 |

TABLE 14
Summary of Treatments

| | | Dose | | | |
|---|---|---|---|---|---|
| Group | Treatment | (mg/kg) | n | MC38 | Strain |
| 1. Iso | Anti-mouse IgG2a | 30 | 8 | + | C57BL/6N |
| 2. aCD38 | Anti-CD38 NIMR-5 | 30 | 5 | + | C57Bl/6N |
| | mouse IgG2a | | | | |
| | (TeneoBio) | | | | |
| 3 silent aCD38 | Silent anti-CD38 NIMR-5 | 10 | 3 | + | C57Bl/6N |
| | mouse IgG2σ | | | | |
| | (in house, JBIO) | | | | |
| 4. silent aCD38 | Silent anti-CD38 NIMR-5 | 30 | 3 | + | C57Bl/6N |
| | mouse IgG2σ | | | | |
| | (in house, JBIO) | | | | |
| 5. Iso CD38 KO | Anti-mouse IgG2a | 30 | 6 | + | C57Bl/6N |
| | | | | | CD38^{-/-} |

The teachings of all patents, published applications and references cited herein are incorporated by reference in their entirety.

What is claimed is:

1. A method of treating a disease in a subject in need thereof, comprising administering to the subject an anti-CD38 antibody in combination with a poly ADP ribose polymerase inhibitor (PARPi), an adenosine receptor antagonist, or both for a time sufficient to treat the disease.

2. The method of claim 1, wherein the anti-CD38 antibody comprises:
a) a heavy chain complementarity determining region 1 (HCDR1), HCDR2 and HCDR3 amino acid sequences of SEQ ID NOs: 6, 7 and 8, respectively; and
b) a light chain complementarity determining region 1 (LCDR1), LCDR2 and LCDR3 amino acid sequences of SEQ ID NOs: 9, 10 and 11, respectively.

3. The method of claim 2, wherein the anti-CD38 antibody is of the IgG1/κ subtype.

4. The method of claim 2, wherein the anti-CD38 antibody comprises:
a) a heavy chain variable region (VH) sequence of SEQ ID NO: 4; and
b) a light chain variable region (VL) sequence of SEQ ID NO: 5.

5. The method of claim 4, wherein the anti-CD38 antibody comprises a heavy chain sequence of SEQ ID NO: 12 and a light chain sequence of SEQ ID NO: 13.

6. The method of claim 1, wherein the anti-CD38 antibody is administered intravenously or subcutaneously.

7. The method of claim 1, comprising administering to the subject an anti-CD38 antibody in combination with a PARPi for a time sufficient to treat the disease.

8. The method of claim 7, wherein the PARPi comprises a PARP1 inhibitor, a PARP2 inhibitor, a PARP3 inhibitor, or a combination thereof.

9. The method of claim 7, wherein the PARPi comprises AZD2461, CEP-8983, CEP-9722, E7016 (GPI21016), Iniparib (BSI 201), INO-1001, Niraparib (MK-4827), Olaparib (AZD-2281), Pamiparib (BGB-290), Rucaparib (AG-014699, PF-01367338), Talazoparib (BMN-673), Veliparib (ABT-888), or a combination thereof.

10. The method of claim 9, wherein the PARPi comprises Niraparib (MK-4827), Olaparib (AZD-2281), Rucaparib (AG-014699, PF-01367338), Talazoparib (BMN-673), or a combination thereof.

11. The method of claim 7, wherein the anti-CD38 antibody and the PARPi are administered separately.

12. The method of claim 1, comprising administering to the subject an anti-CD38 antibody in combination with an adenosine receptor antagonist for a time sufficient to treat the disease.

13. The method of claim 12, wherein the adenosine receptor antagonist comprises an A₁ receptor (A₁AR) antagonist, an A_{2A} receptor (A_{2A}AR) antagonist, an A_{2B} receptor (A_{2B}AR) antagonist, an A₃ receptor (A₃AR) antagonist, or a combination thereof.

14. The method of claim 13, wherein the adenosine receptor antagonist comprises:
a) an A₁AR antagonist selected from the group consisting of BG 9719, DPCPX, FK453, FR194921, N-0861, rolofylline (KW 3902), tonapofylline (BG 9928), WRC-0571, and combinations thereof;
b) an A_{2A}AR antagonist selected from the group consisting of caffeine, 8-(-3-chlorostyryl)-caffeine (CSC), istradefylline (KW-6002), Preladenant (SCH 420814), SCH 58261, SCH 442416, SYN115, VER 6947, VER 7835, ZM241,385, a compound having the structure of: 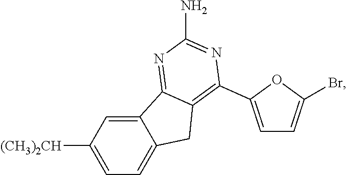
and combinations thereof;
c) an A_{2B}AR antagonist selected from the group consisting of MRE 2029-F20, MRS1754, OSIP-339391, a compound having the structure of: 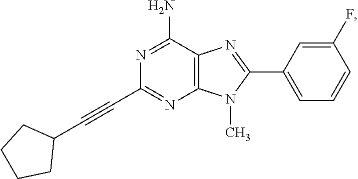 [SMILES: Cn1c(-c2cccc(F)c2)nc2c(N)nc(C#CC3CCCC3)nc21]
and combinations thereof;
d) an A₃AR antagonist selected from the group consisting of FA385, MRE 3008-F20, MRS1292, MRS1334, MRS1523, MRS3777, OT-7999, PSB-11, VUF5574, a compound having the structure of: 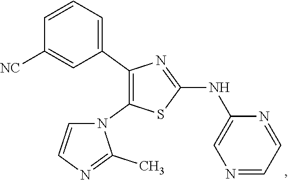 [SMILES: Cc1nccn1-c1sc(Nc2cnccn2)nc1-c1cccc(C#N)c1]
and combinations thereof, or
a combination of a) to d).

15. The method of claim 12, wherein the anti-CD38 antibody and the adenosine receptor antagonist are administered separately.

16. The method of claim 1, comprising administering to the subject an anti-CD38 antibody in combination with a PARPi and an adenosine receptor antagonist for a time sufficient to treat the disease.

17. The method of claim 1, wherein the disease is a CD38-positive hematological malignancy, a cancer, a neurological disorder or a liver disease.

18. The method of claim 17, wherein the CD38-positive hematological malignancy is multiple myeloma (MM), acute lymphoblastic leukemia (ALL), non-Hodgkin's lymphoma (NHL), diffuse large B-cell lymphoma (DLBCL), Burkitt's lymphoma (BL), follicular lymphoma (FL), mantle-cell lymphoma (MCL), acute myeloid leukemia (AML) or chronic lymphocytic leukemia (CLL).

19. The method of claim 17, wherein the cancer is a hematologic cancer or a solid tumor.

20. The method of claim 17, wherein the neurological disorder is acute spinal cord injury (SCI), Alzheimer's Disease (AD), amyotrophic lateral sclerosis (ALS), ataxia, Bell's palsy, a brain tumor, cerebral aneurysm, epilepsy, Guillain-Barré syndrome (GBS), hydrocephalus, a lumbar disk disease, meningitis, multiple sclerosis (MS), muscular dystrophy, a neurocutaneous syndrome, Parkinson's disease (PD), stroke, a cluster headache, a tension headache, a migraine headache, encephalitis, septicemia or myasthenia gravis (MG).

21. The method of claim 17, wherein the liver disease is alagille syndrome (ALGS), autoimmune hepatitis (AIH), biliary atresia, cirrhosis, hemochromatosis, hepatitis, nonalcoholic fatty liver disease (NAFLD), primary biliary cholangitis (PBC), primary sclerosing cholangitis (PSC) or Wilson disease (WD).
